(19) United States
(12) Patent Application Publication
(10) Pub. No.: US 2009/0162363 A1
(43) Pub. Date: Jun. 25, 2009

(54) PEDF-R RECEPTOR AND USES

(76) Inventors: Sofia Patricia Becerra, Bethesda, MD (US); Luigi Notari, North Bethesda, MD (US); Jorge Laborda, Albacete (ES); Julio Escribano-Martinez, Albacete (ES)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 10/566,540

(22) PCT Filed: Aug. 5, 2004
(86) PCT No.: PCT/US04/25560

§ 371 Date: Jan. 22, 2008

Related U.S. Application Data
(60) Provisional application No. 60/493,713, filed on Aug. 7, 2003.
(60) Provisional application No. 60/579,177, filed on Jun. 12, 2004.

(51) Int. Cl. A61K 39/395; C07H 21/04; C07K 14/705; C12N 15/63; C12N 5/10; C12P 21/02; C07K 16/28; A61K 48/00; A61K 31/7088; A61P 25/00; C12N 5/16; G01N 33/566; C12Q 1/68; A61K 38/17

(52) U.S. Cl. 424/139.1; 536/24.5; 536/23.5; 530/350; 435/320.1; 435/325; 435/69.1; 530/389.1; 435/331; 530/388.22; 436/501; 435/6; 514/12; 514/44

(57) ABSTRACT

A pigment epithelium derived factor (“PEDF”) receptor designated PEDF-R and PEDF-R encoding nucleic acid and amino acid sequences. Wild type PEDF-R, PEDF-R variants, soluble PEDF-R variants, chimeric PEDF-R, and antibodies which bind to the PEDF-R (including agonist and neutralizing antibodies), as well as various uses for these molecules are described. Assay systems for detecting ligands to PEDF-R, systems for studying the physiological role of PEDF-R and its ligands, diagnostic techniques for identifying PEDF-related conditions, therapeutic techniques for the treatment of PEDF-related and PEDF-R related conditions, and methods for identifying molecules homologous to PEDF-R.

### CROSS REFERENCE TO RELATED APPLICATIONS

This application claims priority, under 35 USC § 119(e), to U.S. Application Nos. 60/579,177, filed Jun. 12, 2004, and 60/493,713, filed Aug. 7, 2003, the disclosures of which are incorporated by reference in their entireties.

### GOVERNMENT RIGHTS

The work leading to the disclosed inventions was funded in whole or in part with Federal funds from the National Institutes of Health, under Project No. 1Z01EY000306-09. Accordingly, the U.S. Government has rights in these inventions.

### FIELD

The present invention relates to a pigment epithelium derived factor (“PEDF”) receptor designated PEDF-R and provides for PEDF-R encoding nucleic acid and amino acid sequences. In particular, the invention relates to wild type PEDF-R, PEDF-R variants, soluble PEDF-R variants, chimeric PEDF-R, and antibodies which bind to the PEDF-R (including agonist and neutralizing antibodies), as well as various uses for these molecules. It also relates to assay systems for detecting ligands to PEDF-R, systems for studying the physiological role of PEDF-R and its ligands, diagnostic techniques for identifying PEDF-related conditions, therapeutic techniques for the treatment of PEDF-related and PEDF-R related conditions, and methods for identifying molecules homologous to PEDF-R.

### BACKGROUND

Many types of neurons depend upon the availability of special regulatory molecules, known as neurotrophic factors, for their survival and well-being. The best characterized of the neurotrophic factors is nerve growth factor (NGF). NGF regulates the survival and specialized function of sympathetic and dorsal root ganglion neurons in the peripheral nervous system and of some cholinergic neurons in the central nervous system. Trophic factors, which act on other neurons, have also been identified, and two such factors, ciliary neurotrophic factor (CNTF) and brain-derived neurotrophic factor (BDNF) have been purified. Moreover, it has recently been shown that some growth factors, such as fibroblast growth factor (FGF) and epidermal growth factor (EGF), which initially were identified based on their mitogenic effects upon cells, also function as survival-promoting agents for some neurons. Post-synaptic target cells and satellite cells, such as glial cells, appear to be major sources of neurotrophic factors.

It has been proposed that the survival of retinal photoreceptor cells can also be regulated by specific neurotrophic factors. Evidence supporting this concept includes the observation that photoreceptors undergo developmental neuronal death in some species, a phenomenon which is generally considered to reflect the limited availability of neurotrophic factors. Photoreceptor development, as well as maintenance of normal function, has also been shown to require interactions with the retinal pigment epithelium (RPE), suggesting that RPE-derived molecules or factors could be necessary for photoreceptor function and survival.

The RPE develops in advance of and lies adjacent to the neural retina. A closed compartment between the two cell layers contains the interphotoreceptor matrix, and many soluble secretory products of RPE and neural retina cells are contained in the interphotoreceptor matrix. Nutrients, metabolites or trophic factors exchanged between the RPE and neural retina, must pass through the interphotoreceptor matrix. RPE cells, for example, are thought to synthesize and secrete a photoreceptor survival-promoting factor (PSPA).

The neural-derived RPE forms a monolayer of cells interposed between the neural retina and circulating blood within the choroid. In this strategic location, the RPE forms a part of the blood-retina barrier, performs functions essential to retinal integrity and functions, and plays important roles in vascular, inflammatory, degenerative, and dystrophic diseases of the retina and choroid. The functions of the RPE in relation to the visual process are several-fold and include light-dark adaptation, phagocytosis of shed photoreceptor outer segment membranes and nutrition. On the other hand, the close interdependence of the RPE and the neural retina during normal development has been known for a long time, but functionally is not well understood, although it is known that the RPE is important for retinal regeneration. It has been consistently observed that loss of contact of the neural retina with the RPE of many vertebrates (retinal detachment) results in degeneration of the retina. As a side effect of the retinal detachment, strong cell proliferation, originating from the RPE which underlies the areas of detachment, has often been observed.

Pigment epithelium derived factor (“PEDF”), a multifaceted neurotrophic factor was first identified in conditioned medium from fetal human retinal pigment epithelium cell culture. It has since been identified as a member of the serpin family of serine protease inhibitors. The mammalian serine protease inhibitors (serpins) are a superfamily of single chain proteins that contain a conserved structure of approximately 370-420 amino acids and generally range between 50 and 100 kDa in molecular mass. The majority of serpins function as protease inhibitors and so are involved in regulation of several proteinase-activated physiological processes, such as blood coagulation, fibrinolysis, complement activation, extracellular matrix turnover, cell migration and prohormone activation. Serpins inhibit proteolytic events by forming a 1:1 stoichiometric complex with the active site of their cognate proteinases, which is resistant to denaturants. The identification of new Serpin polypeptides permits the development of a range of derivatives, agonists and antagonists at the nucleic acid and protein levels which in turn have applications in the treatment and diagnosis of a range of conditions such as blood coagulation, fibrinolysis, complement activation, extracellular matrix turnover, cell migration and prohormone activation. Gettins, et al., Biol. Chem., 383: 1677-1682, 2002; Potempa, et al., J. Biol. Chem., 269: 15957-19560, 1994; Cohen, et al., Biochemistry, 17: 392-400, 1987.

Although a member of the serpin family of mainly serine protease inhibitors, many of PEDF's effects, e.g., neurotrophic, neuronotrophic, antiangiogenic and gliastatic effects, are independent from serpin activity. PEDF shares folding activity with the serine protease inhibitors, but has some very different activities, for example, PEDF acts in neuronal survival and differentiation in the retina and CNS. It also acts in excluding vessels from invading the retina, vitreous, and aqueous, as well as vessels from nourishing tumors.

Neurotrophic factors such as PEDF have been proposed as potential means for enhancing specific neuronal cell survival, for example, as a treatment for neurodegenerative diseases such as amyotrophic lateral sclerosis, Alzheimer's disease, stroke, epilepsy, Huntington's disease, Parkinson's disease, and peripheral neuropathy. Protein neurotrophic factors, or neurotrophins, which influence growth and development of the vertebrate nervous system, are believed to play an important role in promoting the differentiation, survival, and function of diverse groups of neurons in the central nervous system and periphery. Neurotrophic factors are believed to have important signaling functions in neural tissues, based in part upon the precedent established with nerve growth factor (NGF). NGF supports the survival of sympathetic, sensory, and basal forebrain neurons both in vitro and in vivo. Administration of exogenous NGF rescues neurons from cell death during development. Conversely, removal or sequestration of endogenous NGF by administration of anti-NGF antibodies promotes such cell death. Heumann, et al., J. Exp. Biol., 132: 133-150, 1987; Hefti, et al., J. Neurosci., 6: 2155-2162, 1986; Thoenen, et al., Annu. Rev. Physiol., 60: 284-335, 1980.

Among its many other activities, PEDF is a potent extracellular neuronal differentiation and survival factor for cells derived from the retina and CNS. It is known to induce neuronal differentiation in retinoblastoma cells, protects retinal neurons, e.g., photoreceptors, from death by apoptosis and other insults, and has a morphogenetic effect on photoreceptor cells. PEDF has neurotrophic effect on neurons from areas including the cerebellum, hippocampus and spinal cord. The PEDF gene spans about 16 kb of DNA and contains 8 exons. It has been mapped to human chromosome 17p13, a part of the chromosome involved in retinal degenerative disease caused by a loss of photoreceptor function and resulting in vision loss. These diseases include retinitis pigmentosa, leber's congenital amaurosis and cone-rod dystrophy. PEDF has an effect in the treatment of all of these diseases and conditions. Biochemically, PEDF is a 50 kDa glycoprotein with high binding affinity to cell surface receptors in human retinoblastoma cells which is mediated by interactions between PEDF polypeptide and extracellular domains of the protein. Blockage of the binding interactions of PEDF has many effects, including neurotrophic ones. PEDF also has binding affinity, albeit lower affinity, for other molecules, including glycosaminoglycans, including heparin, heparin- and chondroitin-sulfates. The PEDF gene and protein sequences can be found in, for example, U.S. Pat. Nos. 6,319,687, 6,451,763, 5,840,686 and WO publication 05/33480. Tink, et al., Nature Reviews Neuroscience, 4: 628-636, 2003; Alberdi, et al., BMC Biochemistry, 4: 1-9, 2003; Gettins, et al., Biol. Chem., 383: 1687-1682, 2002; Alberdi, et al., Journal of Biological Chemistry, 274: 31605-31612, 1999; Aymerich, et al., Investigative Ophthalmology & Visual Science, 42: 3287-3293, 2001.

The aberrant expression or uncontrolled regulation of any one of neurotrophic factor receptors, such as PEDF-R, can result in different malignancies and pathological disorders. Therefore, there exists a need to identify means to regulate, control and manipulate PEDF-R and their associated ligands, in order to provide new and additional means for the diagnosis and therapy of PEDF-related disorders and cellular processes. The present meets this and other needs.

### SUMMARY

The present invention relates to a transmembrane receptor with binding affinity to PEDF termed the PEDF receptor or “PEDF-R”. In particular, it relates to a polynucleotide comprising a coding sequence for PEDF-R, a polynucleotide that selectively hybridizes to the complement of a PEDF-R coding sequence, expression vectors containing such polynucleotides, genetically engineered host cells containing such polynucleotides, PEDF-R polypeptides, PEDF-R fusion proteins, therapeutic compositions, PEDF-R domain mutants, antibodies specific for PEDF-R polypeptides, methods for detecting the expression of PEDF-R, methods of modulating PEDF-R expression and activity, and methods of modulating PEDF activity. A wide variety of uses are encompassed by the invention including, but not limited to, treatment of neurological diseases and disorders; ocular diseases and disorders; diseases and disorders caused by angiogenesis; and obesity-related disorders (by prevention of lipid accumulation, an indication that is known to occur in several retinal pathologies, including, but not limited to, age-related macular degeneration referred to as AMD, diabetic retinopathy, and the like).

In one aspect, the invention provides an isolated PEDF-R polynucleotide, that is (a) a polynucleotide that comprises the sequence of SEQ ID NO: 1, 2 or 4; (b) a polynucleotide that hybridizes under stringent hybridization conditions to (a) and encodes a polypeptide having the sequence of SEQ ID NO: 3 or 5; (c) a polynucleotide that hybridizes under stringent hybridization conditions to (a) and encodes a polypeptide with at least 25 contiguous residues of the polypeptide of SEQ ID NO: 3 or 5; or (d) a polynucleotide that hybridizes under stringent hybridization conditions to (a) and has at least 12 contiguous bases identical to or exactly complementary to SEQ ID NO: 1, 2, or 4, wherein the polynucleotide encodes a polypeptide having PEDF-R activity.

In one aspect, the invention provides an isolated PEDF-R polynucleotide, that is (a) a polynucleotide that comprises the sequence of SEQ ID NO: 12, 13, 15 or 16; (b) a polynucleotide that hybridizes under stringent hybridization conditions to (a) and encodes a polypeptide having the sequence of SEQ ID NO: 14 or 17; (c) a polynucleotide that hybridizes under stringent hybridization conditions to (a) and encodes a polypeptide with at least 25 contiguous residues of the polypeptide of SEQ ID NO: 14 or 17; or (d) a polynucleotide that hybridizes under stringent hybridization conditions to (a) and has at least 12 contiguous bases identical to or exactly complementary to SEQ ID NO: 12, 13, 15 or 16, wherein the polynucleotide encodes a polypeptide having PEDF-R activity.

In one aspect the invention provides an isolated PEDF-R polynucleotide encoding a polypeptide comprising a sequence at least 60% identical to SEQ ID NO:3, SEQ ID NO:5, SEQ ID NO:14, or SEQ ID NO:17 and having PEDF-R activity. In one embodiment, the invention provides an isolated PEDF-R polynucleotide encoding a polypeptide comprising the sequence of SEQ ID NO:3, 5, 14 or 17. In a related aspect, the present invention provides a PEDF-R polynucleotide encoding a polypeptide that specifically binds to amino acids 44-121 of PEDF. In a related aspect, the present invention provides a PEDF-R polynucleotide encoding a polypeptide that specifically binds to amino acids 78-121 of PEDF. In a related aspect, the present invention provides a PEDF-R polynucleotide encoding a polypeptide that specifically binds to amino acids 44-77 of PEDF. In an embodiment of the present invention, the provided PEDF-R polynucleotide encodes a polypeptide having a binding affinity of at least 10⁴ M⁻¹ for binding PEDF. The invention also provides functional fragments and conservatively modified variants of SEQ ID NOS. 3, 5, 14 or 17 wherein said functional fragments and conservatively modified variants have PEDF-R activity. The invention also provides nucleic acid molecules encoding functional fragments of a polypeptide comprising SEQ ID NOS. 3, 5, 14 or 17 or conservatively modified variants of a polypeptide comprising SEQ ID NOS. 3, 5, 14 or 17 wherein said functional fragments and conservatively modified variants have PEDF-R activity (e.g., specifically bind to PEDF).

In one aspect, the present invention provides a PEDF-R polynucleotide comprising SEQ ID NO:1, 2, 4, 12, 13, 15 or 16 or a complement thereof.

In one aspect, the present invention an isolated polynucleotide comprising a nucleotide sequence having at least 60% identity to SEQ ID NO:1, 2, 4, 12, 13, 15, 16 or complement thereof and having PEDF-R activity. In one embodiment, the invention provides an isolated polypeptide comprising a nucleotides sequence that has at least 90% sequence identity to SEQ ID NO:3, SEQ ID NO:5, SEQ ID NO:14, or SEQ ID NO:17 and is immunologically cross-reactive with SEQ ID NO:3, SEQ ID NO:5, SEQ ID NO:14, or SEQ ID NO:17 or shares a biological function with native PEDF-R.

In another aspect, the present invention provides vectors, such as expression vectors, comprising a polynucleotide sequence of the invention. In other embodiments, the present invention provides host cells or progeny of the host cells comprising a vector of the invention. In certain embodiments, the host cell is a eukaryote. In other embodiments, the expression vector comprises a PEDF-R polynucleotide in which the nucleotide sequence of the polynucleotide is operatively linked with a regulatory sequence that controls expression of the polynucleotide in a host cell. In certain embodiments, the invention provides a host cell comprising a PEDF-R polynucleotide, wherein the nucleotide sequence of the polynucleotide is operatively linked with a regulatory sequence that controls expression of the polynucleotide in a host cell, or progeny of the cell. The nucleotide sequence of the polynucleotide can be operatively linked to the regulatory sequence in a sense or antisense orientation.

In another aspect, the invention provides a PEDF-R polynucleotide that is an antisense polynucleotide. In a preferred embodiment, the antisense polynucleotide is less than about 200 bases in length. In other embodiments, the invention provides an antisense oligonucleotide complementary to a messenger RNA comprising SEQ ID NO:1, 2, 4, 12, 13, 15, or 16 and encoding PEDF-R, wherein the oligonucleotide inhibits the expression of PEDF-R.

In another aspect, the present invention provides an isolated DNA that encodes a PEDF-R protein as shown in SEQ ID NO: 3, 5, 14, or 17. In certain embodiments, the PEDF-R polynucleotide is RNA.

The present invention provides a method of producing a polypeptide comprising (i) culturing a host cell of the present invention under conditions such that the polypeptide is expressed; and (ii) recovering the polypeptide from the cultured host cell of its cultured medium.

The invention further provides an isolated PEDF-R polypeptide encoded by a PEDF-R polynucleotide. In some embodiments, the PEDF-R polypeptide has 60% sequence identity to the amino acid sequence of SEQ ID NO:5 and has PEDF-R activity. In some embodiments, the PEDF-R polypeptide comprises the amino acid sequence of SEQ ID NO:3, 5, 14, or 17. In some embodiments, the isolated PEDF-R polypeptide is cell-membrane associated. In other embodiments, the isolated PEDF-R polypeptide is soluble. In one aspect, the PEDF-R polypeptide is fused with a heterologous polypeptide.

The present invention further provides an isolated antibody or antibody composition that specifically binds to a polypeptide having the amino acid sequence as shown in SEQ ID NO:3′ SEQ ID NO:5, SEQ ID NO:14 or SEQ ID NO:17. In some embodiments, the antibody is monoclonal. In other embodiments, the antibody is polyclonal. In some embodiments, the antibodies of the present invention are labeled. In some embodiments, the isolated antibodies of the present invention are conjugated to a toxic or non-toxic moiety. In one aspect, the isolated antibodies of the present invention are neutralizing antibodies. In one embodiment, the invention provides hybridomas capable of secreting the antibodies of the present invention.

In addition to PEDF-R's ability to act as a transmembrane receptor with affinity to PEDF and to modulate PEDF as well as other PEDF-R ligands, it is also contemplated that PEDF-R plays a role in the transport and secretion of PEDF-R ligands across cell membranes, for example, the retinal pigment epithelium (RPE).

The present invention further provides a method for identifying a compound or agent that binds to a PEDF-R polypeptide comprising (i) contacting a PEDF receptor polypeptide with the compound or agent under conditions which allow binding of the compound to the PEDF-R polypeptide to form a complex and (ii) detecting the presence of the complex.

The invention further provides a method of detecting a PEDF-R polypeptide in a sample, comprising (i) contacting the sample with an antibody of the present invention, and (ii) determining whether a hydridization complex has been formed between the antibody and the PEDF-R polypeptide.

The present invention further provides a method of detecting a PEDF-R polypeptide in a sample, comprising (i) contacting the sample with a PEDF-R polynucleotide or a polynucleotide that comprises a sequence of at least 12 nucleotides and is complementary to a contiguous sequence of the PEDF-R polynucleotide and (ii) determining whether a hydridization complex has been formed. In some embodiments, the methods of the present invention are used to diagnose a disease or disorder of the nervous system, a disease or disorder associated with angiogenesis, or an ocular disease or disorder.

The present invention provides a method of detecting a PEDF-R nucleotide in a sample, comprising: (i) using a polynucleotide that comprises a sequence of at least 12 nucleotides and is complementary to a contiguous sequence of a PEDF-R polynucleotide, in an amplification process, and (ii) determining whether a specific amplification product has been formed.

The present invention further provides a pharmaceutical composition comprising a PEDF-R polynucleotide, or a PEDF-R polypeptide or an antibody capable of specifically binding to PEDF-R and a pharmaceutically acceptable carrier.

The present invention further provides a method of modulating PEDF activity in vivo or in vitro, comprising (i) modulating the expression of a PEDF-R gene; (ii) modulating the ability of a PEDF-R protein to bind to another cell; or (iii) modulating the ability of a PEDF-R protein to bind to another protein.

The present invention further provides a method of modulating PEDF activity in a subject, comprising administering to the subject a therapeutically effective amount of a pharmaceutical composition of the present invention. In some embodiments, the PEDF activity is neurotrophic, neuronotrophic, gliastatic, angiogenic, and adipostatic. In one aspect, the PEDF activity is the inhibition of ocular angiogenesis or neovascularizaton. In another aspect, the PEDF activity is ocular angiogenesis caused by ischemia. In another aspect, the PEDF activity is the inhibition of retinal cell degeneration. In a further aspect, the PEDF activity is the prevention of the accumulation of lipids or leads to the dissipation of the accumulation of lipids, which, in turn, could lead to protecting neuronal cells and photoreceptors from apoptotic death and/or preventing angiogenesis itself.

The present invention provides a method of treating a neurological disease or disorder, an ocular disease or disorder, or a disease or disorder associated with angiogenesis or neovascularization in a subject comprising administering to the subject a therapeutically effective amount of a pharmaceutical composition comprising a pharmaceutical composition of the present invention.

### BRIEF DESCRIPTION OF THE DRAWINGS

FIG. 1. Organization of the Human PEDF-R1 cDNA. A. The ORF is indicated by an open box, the predicted transmembrane (TM) domains by gray boxes (amino acid residues 7-24, 43-63, 140-159, and 325-347) and N-glycosylation sites by ticks at the top (amino acid residues 9, 39, 209 and 425). The hatched box shows the PEDF binding region p12 (amino acid residues 250-383). B. Hydrophobicity plot of the derived amino acid sequence of R1. C. Diagram showing a model for R1 topology. TM positions with preferred orientations were predicted using TMpred software. D. Amino acid sequence derived from human R1 cDNA and its alignment to adiponutrin. Non conserved amino acids sequence is shown for the adiponutrin. A patatin-like region is in bold italic (amino acid residues 7-180) and TM domains are indicated by open boxes. E. Regions of R1 (p12 and C-terminal region) showing similarity with human collagen I (alpha chain). Sequences were aligned using SIM-LALNVIEW software and similarities above a threshold of 25% were considered. The range of identity between p12 regions (253-293) and several areas of human collagen I (alpha chain) is 25-71.4%. The range of identity between a C-terminal regions of R1 (450-504) and several areas of human collagen I (alpha chain) is 25-66.7%. Proline (red) rich regions, typical of collagen chain, are shown. This shows that R1 has similarity to human collagen I in the PEDF binding region (p12) and C-end region. We have shown that PEDF has binding affinity for collagen I. before (Meyer et al., JBC, 277: 45400-7, 2002). This is of interest because it may represent the molecular basis for the binding affinity of R1 for PEDF. F. Alignment of partial sequences around conserved residues of R1, patatin B2 and cytoplasmic cPLA2. Active site residues of cPLA2: Ser228, Asp549, of patatin B2 Ser54 and Asp192. The homologous patatin phospholipase A (PLA) active residues of human R1 correspond to Ser47 (S47) and Asp166 (D166). The sites in Patatin B2 and cPLA2 have been obtained from crystallographic and mutational studies of these proteins (Hirschberg et al., Eur J Biochem, 268: 5037-5044, 2001). X-ray crystallographic data clearly revealed that patatin possessed a Ser-Asp catalytic dyad and an active site similar to that observed in the catalytic domain of human cytosolic cPLA2 (Rydel et al., Biochemistry, 42: 6696-6708, 2003).

FIG. 2. PCR of reverse-transcribed mRNA from retina cells with p12 primers. Polymerase Chain Reaction (PCR) of cDNA samples from retina cells with specific p12 primers. Templates were Ret, human retina; RPE and RPE2, human retinal pigment epithelium; ARPE19 and hTERT, human RPE cells; RGC-5, rat retinal ganglion cells; and R28, rat retinal cells. PCR products were resolved by agarose gel electrophoresis and visualized with ethidium bromide. GADPH primers were used as a control.

FIG. 3. PCR of Reverse-transcribed mRNA from Cell Lines. Polymerase Chain Reaction (PCR) of cDNA samples from cells with specific primers (In2, mIN2, rIn2, p12) as indicated. Templates were p12 plasmid (human); BHK, baby hamster kidney cell line; HUVEC, human umbilical cord vein endothelial cells; human RPE, retinal pigment epithelium; ARPE19, RPE cells; pR1 plasmid (human), mouse NIH 3T3 L1 preadipocytes cell line; mouse NIH 3T3 fibroblasts cell line; RGC-5, rat retinal ganglion cell line; and R28, rat retinal cell line. PCR products were resolved by agarose gel electrophoresis and visualized with ethidium bromide. 18S and GADPH primers were used as controls.

FIG. 4. PCR of Reverse-transcribed RNA of human Tissues with p12 primer. Polymerase Chain Reaction (PCR) of cDNA samples from human tissues with specific p12 primers. Templates were as indicated at the top of the figure. PCR products were resolved by agarose gel electrophoresis and visualized with ethidium bromide. 18S primers were used as a control. Control RNA was from Ambion. Detecton of PEDF-R1 by PCR with p12 primers, was very high in adipose tissue.

FIG. 5. Northern analysis of human adipose and skin RNA for R1. Northern blot of RNA isolated from BHK cells, rat R28 cells, and commercially available RNA from human skin. The Northern was performed following the instructions by Ambion for the Glyo Max gel and transfer to Brightstar membranes. The probe was prepared following instructions by Ambion; Psolaren-biotin label of a p12 PCR fragment (as above). Prehybridization, hybridization and washes were in Brightstar Psolaren-biotin label kit (Ambion). A photograph under UV light of the gel before transfer is shown at the bottom to visualize RNA in gel. The size of the transcript for R1 in adipose tissue was confirmed by Northern and the size was as expected for PEDF-R1 mRNA (from TTS2.2, Accession BC017280, GI: 16878146).

FIG. 6. Constructs of Candidate PEDF Receptor, R1. R1 expression constructs were prepared. Nucleotide sequences of each pEXP1-12N, pEXP1-12C, pEXP1-R1N and pEXP2-R1C vector were confirmed by standard methods.

FIG. 7. Overexpression of p12 and R1 cDNAs in bacterial extracts. Coupled transcription/translation reactions for in vitro protein synthesis were performed in E. coli extracts from expression vectors containing R1 cDNA fragments under the control of the T7 transcriptional promoter (RTS). Western blots of the extracts against Anti-Xpress and anti-V5 are shown. Plasmids used in each reaction are indicated at the top of each lane. pEXP1-LacZ and pEXP2-LacZ are positive controls for the reactions.

FIG. 8. Purification of recombinant R1 polypeptides by Ni-NTA Affinity Column Chromatography. Poros MC column attached to a BioCad 700E computerized system was used. A. Purification of p12 from an IVS reaction mixture with pEXP1-R1N. A chromatogram, SDS-PAGE and western blot vs. anti-His tag (Ab-His) of fractions from the purification are shown. B. Purification of R1 from a RTS reaction mixture with pEXP1-R1N. A chromatogram, SDS-PAGE of fractions from the purification are shown. L, Load; FT, Flow-trough; asterisks indicate peak fractions; arrow indicates migration positions of recombinant proteins.

FIG. 9. Solubility studies on recombinant R1 polypeptides. Western blots showing p12N (A) and R1N (B) present in the soluble and insoluble fractions of a solubility assay (see scheme of the fractionation procedure in C). Reaction mixtures were resuspended in buffers containing increasing concentrations of chaotropic agents or detergents, incubated for 30 min on ice and subjected to centrifugation. The R1 polypeptides detected by Western blot vs. anti-Xpress.

FIG. 10. PEDF binding to R1. Soluble fractions of RTS-500 reaction mixtures containing R1N polypeptides were mixed without or with PEDF in binding buffer. A. Western blot of PEDF immunoreactivity in His-tag pull-down assays. B. Western blot of PEDF immunoreactivity in complex formation assays. Complex formation assays were performed as described by Meyer et al., JBC, 2002.

FIG. 11. PEDF binding to p12. Soluble fractions of IVS reaction mixtures containing p12N polypeptides were mixed without or with PEDF in binding buffer. A. His-tag pull-down assays with p12 and PEDF. Reactions were analyzed by Western blotting against anti-PEDF. B. Solid phase binding assays of purified p12 to immobilized PEDF or BSA. Bound p12 was detected with Anti-HisG-HRP and ELISA Femto by luminescence. LU=arbitrary units for photons detected from the HRP reaction with luminometer.

FIG. 12. Surface Plasmon Resonance. Real Time Surface Plasmon Resonance assays (BIAcore) of the binding of R1 and p12 to PEDF and kinetic analysis for binding of R1 to PEDF. PEDF protein was immobilized on the surface of a CM5 sensor chip; the reference cell was without PEDF. (A) Soluble R1 and p12 polypeptides were analyzed for the real time binding reaction in buffer HBS-N. A plot showing the R.U. of analytes after binding to immobilized PEDF. Controls minus R1 peptides, were LacZ, fractions of Ni-NTA column chromatography with out R1 peptides. (B) Kinetic analysis of binding of R1N to PEDF in PBS buffer containing 0.1% NP-40. Kinetic parameters for R1-PEDF interaction are reported on table in (C).

FIG. 13. Phospholipase activity in R1. A. Scheme of phospholipase A (PLA) activity assay. PLA substrate, [1,2-dilinoleoyl]-phosphatidilcoline; coupling enzyme, lipoxygenase as. PLA catalyses the release of linoleic acid, which is oxidized by lipoxygenase, forming a derivative hydroperoxide that is detected at 234 nm as a result of the formation of the linoleic acid hydroperoxide. A scan of the products formed every minute for 10 min is shown. B. Phospholipase A activity of R1. R1N was the soluble fraction from in vitro protein synthesis (RTS-500) in 3 mM DOC, Tris-C1 pH 7.5. B. Reaction mixtures were in 3 mM DOC, 50 mM Tris-C1 pH 7.5. The reaction rates (Y axis, ΔAbsorbance @ 234 nm/min), are plotted for each PLA2 and R1N assay. The concentration of PLA2 activity was estimated from the activity of the commercial PLA2 by comparing the known dA/min of the commercial PLA (shown) with the activity of the R1N fraction. C. Optimization of the PLA activity in R1. R1N was resuspended in Tris or Borate Buffer at the indicated pH. The soluble fractions were separated by centrifugation and assayed for PLA activity.

FIG. 14. Subcellular localization of R1. Immunofluorescence (A) and confocal imagery (B) analyses with epitope-tagged protein transiently expressed in COS-7 and retina RCG-5 cells show that PEDF-R1 localizes to membranes.

FIG. 15. Fractionation of COS-7 cells transiently transfected with pLumio-R1N DNA plasmids. The fractionation of cells into cytosolic and plasma membranes was performed as described before by Aymerich et al., IOVS 1999. In brief, harvested cells were washed by centrifugation with PBS and homogenization buffer was added. After homogenization and sonication, debris was separated by low speed centrifugation. The soluble material was further subjected to high speed centrifugation to fractionate plasma membranes in the pellet. The pellet was resuspended in SDS-sample buffer. Protein concentration was determined in the fractions and then equal amount loaded on gels for further analysis. Alternatively, Lumio reagent was detected as described by Invitrogen in gels using Laser scanning (Typhoon). Note the Lumio reagent fluoresces as the biarsenical reagent reacts with tetracysteines in the Lumio-tag fused to R1 (see www.invitrogen.com). Proteins were transferred to nitrocellulose membranes and stained with Ponceau Red. Immunostaining using Anti-V5 antibody was performed as described above (Invitrogen's instructions).

FIG. 16. Complex formation assays between PEDF and R1. Complex formation assays were performed with PEDF and the cytosolic fractions from COS-7 cells transiently expressing R1 using centricon-100 devices, as described by Meyer et al., JBC, 277: 45400-7, 2002. This shows that PEDF binds to Lumio-R1N protein from a mammalian system.

FIG. 17. Effect of PEDF on the expression of PEDF-R1RNA. Human ARPE-19 and mouse NIH3T3-L1, rat R28 and rat RGC-5 cells were cultured in media containing serum (FBS). Media was replaced without serum and plus and minus 50 nM human recombinant PEDF protein. A. The NIH3T3-L1 and ARPE-19 cells were incubated for 24 hours before harvesting. B. The R28 and RGC-5 cells were incubated for 36 hours before harvesting. C. The run curve depicts the fluorescence of the sample (ARPE-19-FBS, +50 nM PEDF) after every cycle in the amplification reaction, showing the relative amounts of R1 expression for each sample. D. The sample temperature was increased in 0.5° C. increments and the fluorescence of the sample was measured at each increment. The downward sloping line represents the melting of the PCR product, and the peak curve is the derivative of the melting curve. The single peak in each sample shows that one PCR product was amplified in each sample. R1 expression is downregulated in NIH3T3-L1 and ARPE-19 cells grown with serum or in the presence of PEDF as compared to cells grown in the absence of both serum and PEDF. The decrease in PEDF levels between cells without and with PEDF treatment is a constant ratio of 1.6 in the studied cell lines. However, R1 expression was upregulated in neural retina precursors R28 and RGC-5 cells cultured with serum, but not with PEDF as compared to cells grown in the absence of serum.

FIG. 18. The role PEDF has on adipogenesis was investigated using an established assay (from Chemicon) in which differentiation of NIH3T3-L1 preadipocytes to mature adipocytes is induced with dexamethasone, isobutylmethylxanthine (IBMX) and insulin to accumulate intracellular lipids. Staining the cells with Oil Red O can reveal the intracellular lipid droplet accumulation under the microscope. Quantification can be accomplished by measuring the extracted lipid stain spectrophotometrically. We found that exogenous additions of PEDF at 5 and 50 nM to the cultures decreased the Oil Red O staining in the cells and the absorbance of the extracts similar to the effects of the cytokine transforming growth factor beta (TGF beta). This suggested that PEDF might interfere with lipid accumulation in cells undergoing adipocyte maturation.

FIG. 19. FIG. 19 provides the amino acid alignment of the mouse (Accession number BAC27476.1), rat (Accession number XP_{—}341961.1) and human (Accession number AAH17280.1) PEDF-R protein.

FIG. 20. FIG. 20 provides the nucleic acid alignment of the mouse (Accession number AK031609.1; chromosome 7), rat (Accession number XM_{—}341960.1; chromosome 1) and human (Accession number BC017280.1; chromosome 11) PEDF-R cDNA.

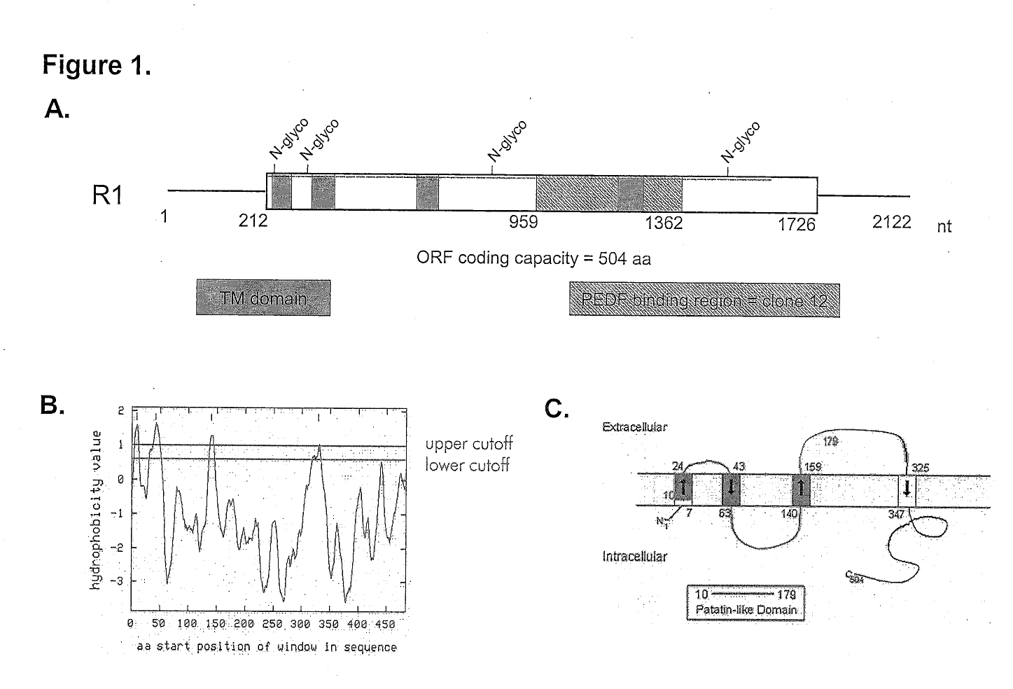

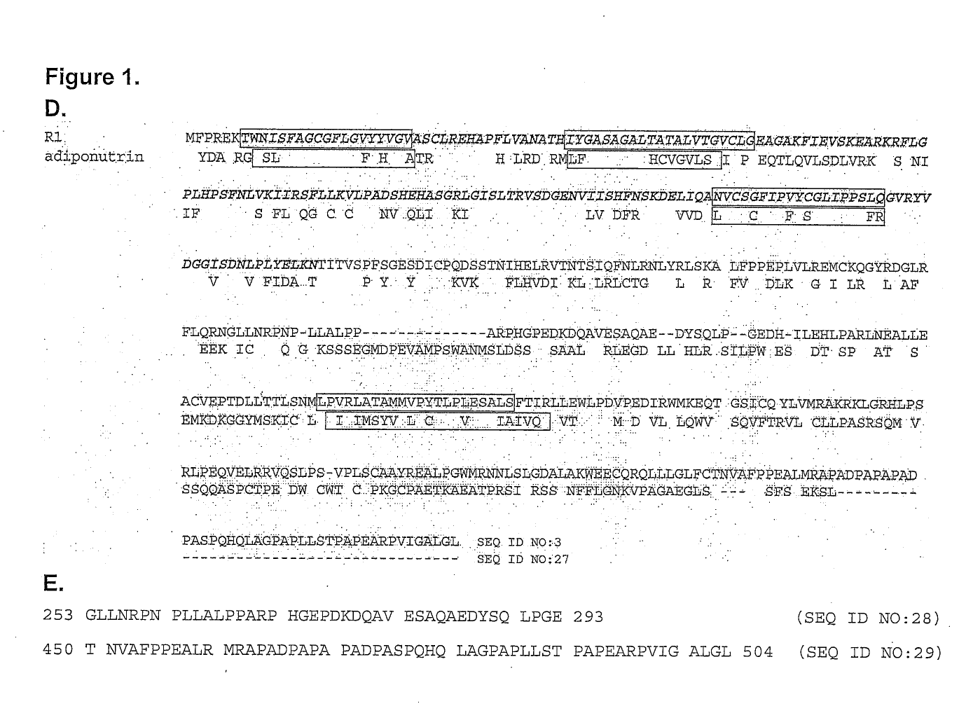

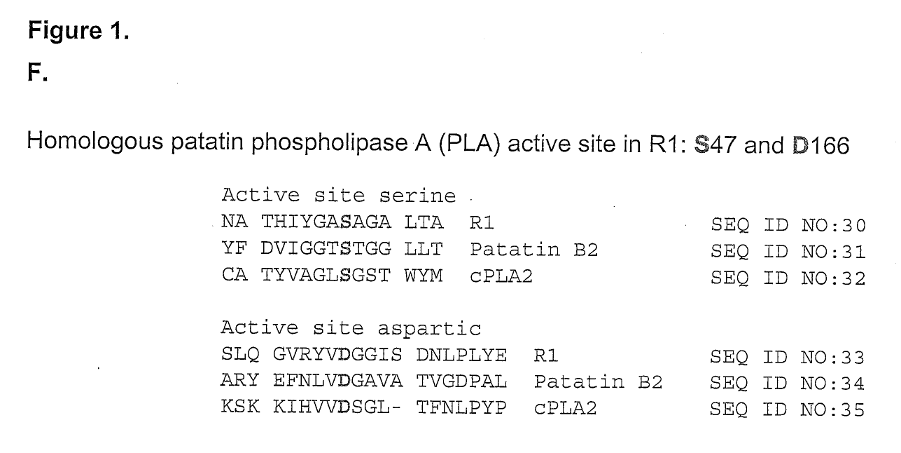

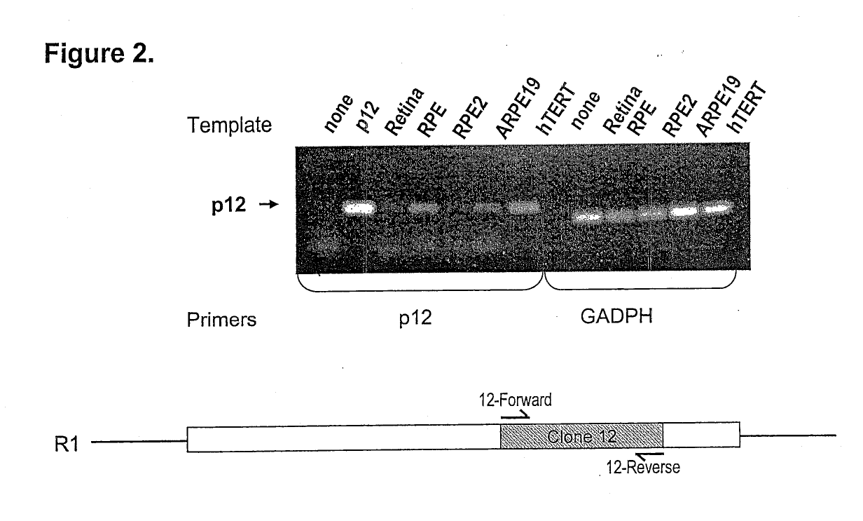

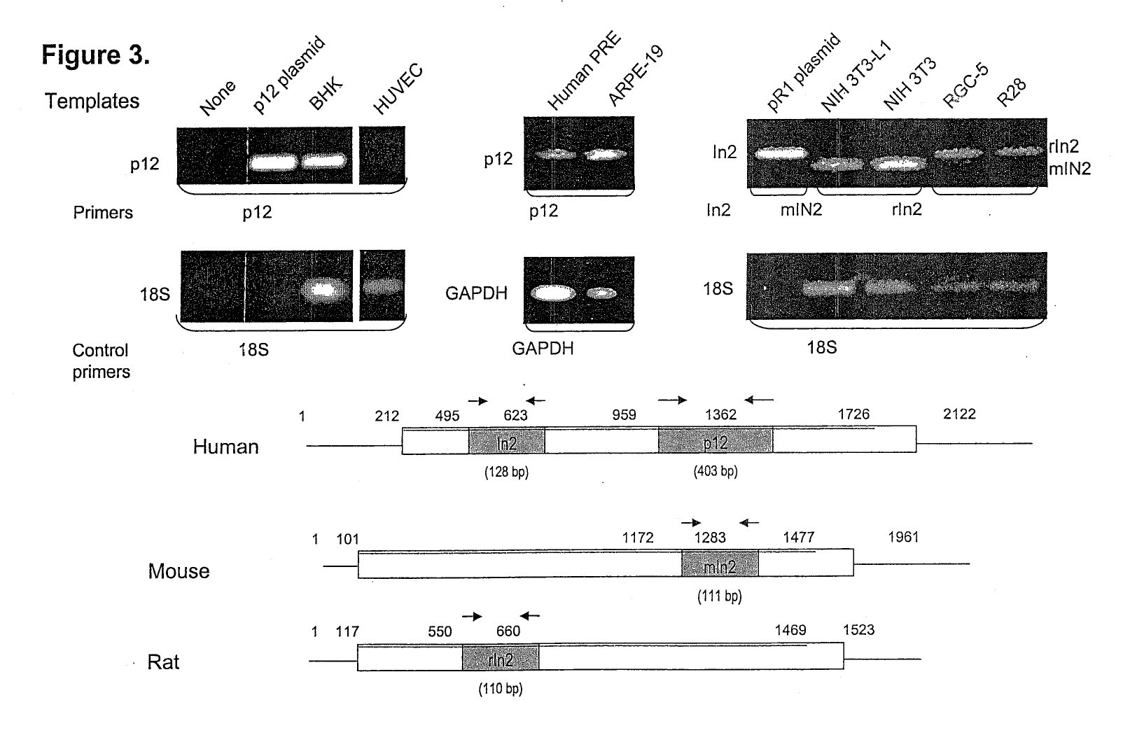

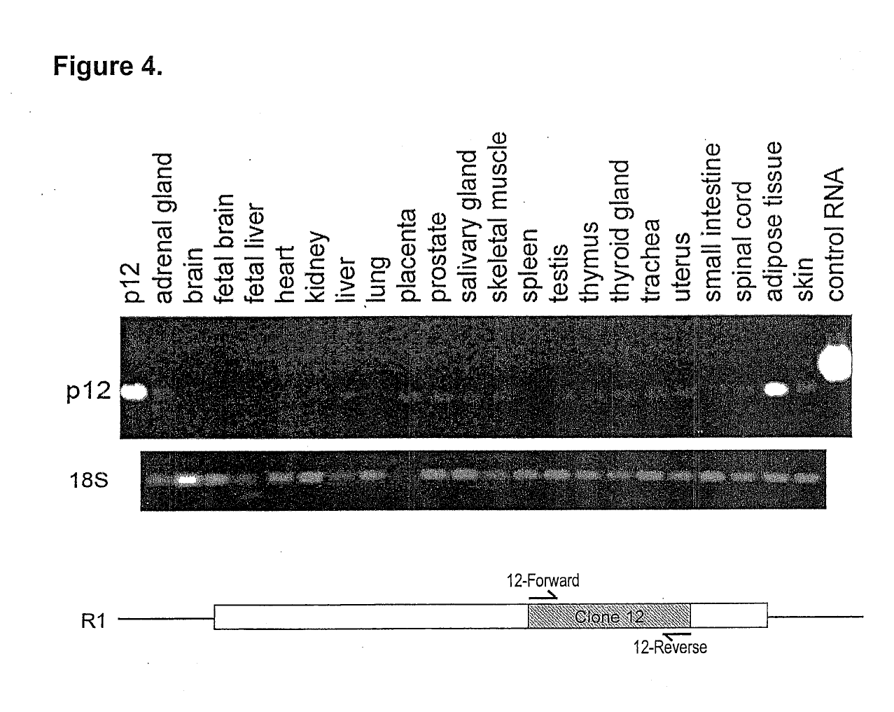

### DETAILED DESCRIPTION

The present invention provides PEDF-R nucleic acid and amino acid sequences. The terms “PEDF-R” or “PEDF-R polypeptide” when used herein encompass wild type PEDF-R; PEDF-R variants; PEDF-R extracellular domain; and chimeric PEDF-R (each of which is defined herein).

PEDF-R refers to a polypeptide that is a transmembrane receptor with binding affinity to PEDF. The term PEDF-R therefore refers to polymorphic variants, alleles, mutants, and interspecies homologs that: (1) have substantial identity to the amino acid sequence of SEQ ID NO:3, SEQ ID NO:5, SEQ ID NO:14, or SEQ ID NO:17 (2) bind to antibodies raised against an immunogen comprising an amino acid sequence selected from the group consisting of SEQ ID NO:3, SEQ ID NO:5, SEQ ID NO:14, SEQ ID NO:17 and conservatively modified variants thereof; (3) encoded by a nucleotide sequence that specifically hybridizes under stringent hybridization conditions to a sequence selected from the group consisting of SEQ ID NO:1, SEQ ID NO:2, SEQ ID NO:4, SEQ ID NO:12, SEQ ID NO:13, SEQ ID NO:15, SEQ ID NO:16 and conservatively modified variants thereof; or (4) encoded by a nucleic acid that is amplified by primers that specifically hybridize under stringent hybridization conditions to the same sequence as a primer set consisting of SEQ ID NO:6, SEQ ID NO:7, and SEQ ID NO:8.

Substantial identity of polynucleotide sequences for these purposes normally means sequence identity of at least 25%. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99% compared to a reference sequence using the programs described herein; preferably BLAST using standard parameters, as described below. Accordingly, polynucleotides of the present invention encoding a protein of the present invention include nucleic acid sequences that have substantial identity to the nucleic acid sequences of SEQ ID NO:1, SEQ ID NO:2, SEQ ID NO:4, SEQ ID NO:12, SEQ ID NO:13, SEQ ID NO:15, or SEQ ID NO:16. Polypeptides or proteins of the present invention include amino acid sequences that have substantial identity to SEQ ID NO:3, SEQ ID NO:5, SEQ ID NO:14, or SEQ ID NO:17. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 30% to 100%. More preferred embodiments include at least 30%, 35%, 40%, 45%, 50%, 55%, 60%, 65%, 70%, 75%, 80%, 85%, 90%, 95%, or 99%. Most preferred embodiments include 61%, 62%, 63%, 64%, 65%, 66%, 67%, 68%, 69%, 70%, 71%, 72%, 73%, 74% and 75%. Polypeptides that are “substantially similar” share sequences as noted above except that residue positions which are not identical can differ by conservative amino acid changes.

The invention also relates to nucleic acids that selectively hybridize to exemplified PEDF-R sequences (including hybridizing to the exact complements of these sequences). Selective hybridization can occur under conditions of high stringency (also called “stringent hybridization conditions”), moderate stringency, or low stringency.

“Stringent hybridization conditions” are conditions under which a probe will hybridize to its target subsequence, typically in a complex mixture of nucleic acid, but not to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. In a specific embodiment, a nucleic acid which is hybridizable to a PEDF-R nucleic acid under the following conditions of high stringency is provided: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 8-16 h at 65° C. in prehybridization mixture containing 100 μg/ml denatured salmon sperm DNA and 5−20×10⁶ cpm of ³²P-labeled probe. Washing of filters is done at 65° C. for 15-30 h in a solution containing 2×SSC, 0.1% SDS. This is followed by a wash in 0.2×SSC and 0.1% at 50° C. for 15-30 min before autoradiography.

In another specific embodiment, a nucleic acid, which is hybridizable to a PEDF-R nucleic acid under conditions of moderate stringency is provided. Examples of procedures using such conditions of moderate stringency are as follows: Filters containing DNA are pretreated for 6 h at 55° C. in a solution containing 6×SSC, 5× Denhart's solution, 0.5% SDS and 100 μg/ml denatured salmon sperm DNA. Hybridizations are carried out in the same solution and 5−20×10⁶ cpm ³²P-labeled probe is used. Filters are incubated in hybridization mixture for 12-16 h at 55° C., and then washed twice for 30 minutes at 50° C. in a solution containing 1×SSC and 0.1% SDS. Filters are blotted dry and exposed for autoradiography. Other conditions of moderate stringency which can be used are well-known in the art. Washing of filters is done at 45° C. for 1 h in a solution containing 0.2×SSC and 0.1% SDS.

By way of example and not limitation, procedures using such conditions of low stringency are as follows (see also Shilo, et al., Proc. Natl. Acad. Sci. U.S.A., 78: 6789-6792, 1981): Filters containing DNA are pretreated for 6 h at 40 C in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 g/ml salmon sperm DNA, 10% (wt/vol) dextran sulfate, and 5−20×10⁶ cpm ³²P-labeled probe is used. Filters are incubated in hybridization mixture for 18-20 h at 40° C., and then washed for 1.5 h at 55 C in a solution containing 2×SSC and 0.1% SDS. The wash solution is replaced with fresh solution and incubated an additional 30 minutes at 50-55° C. Filters are blotted dry and exposed for autoradiography. If necessary, filters are washed for a third time at 60-65° C. and reexposed to film. Other conditions of low stringency that can be used are well known in the art (e.g., as employed for cross-species hybridizations).

A “wild type PEDF-R” or “native PEDF-R” comprises a polypeptide having the same amino acid sequence as a PEDF-R derived from nature. Thus, a wild type PEDF-R can have the amino acid sequence of naturally occurring rat PEDF-R, murine PEDF-R, human PEDF-R, or PEDF-R from any other mammalian species. Such wild type PEDF-R polypeptides can be isolated from nature or can be produced by recombinant or synthetic means. The term “wild type PEDF-R” specifically encompasses naturally-occurring truncated forms of the PEDF-R, naturally-occurring variant forms (e.g., alternatively spliced forms), and naturally-occurring allelic variants of the PEDF-R.

The phrase “nucleic acid” or “polynucleotide sequence” refers to a single or double-stranded polymer of deoxyribonucleotide or ribonucleotide bases read from the 5′ to the 3′ end. Nucleic acids can also include modified nucleotides that permit correct read through by a polymerase and do not alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the wild type sequence or sequences which can be introduced to provide codon preference in a specific host cell.

The term “promoter” refers to a region or sequence determinants located upstream or downstream from the start of transcription and which are involved in recognition and binding of RNA polymerase and other proteins to initiate transcription.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants.

“Increased or enhanced expression or activity of a polypeptide of the present invention,” or “increased or enhanced expression or activity of a polynucleotide encoding a polypeptide of the present invention,” refers to an augmented change in activity of the polypeptide or protein. Examples of such increased activity or expression include the following: Activity of the protein or expression of the gene encoding the protein is increased above the level of that in wild-type, non-transgenic controls. Activity of the protein or expression of the gene encoding the protein is in an organ, tissue or cell where it is not normally detected in wild-type, non-transgenic controls (i.e., spatial distribution of the protein or expression of the gene encoding the protein is altered). Activity of the protein or expression of the gene encoding the protein is increased when activity of the protein or expression of the gene encoding the protein is present in an organ, tissue or cell for a longer period than in a wild-type, non-transgenic controls (i.e., duration of activity of the protein or expression of the gene encoding the protein is increased).

“Decreased expression or activity of a protein or polypeptide of the present invention,” or “decreased expression or activity of a nucleic acid or polynucleotide encoding a protein of the present invention,” refers to a decrease in activity of the protein. Examples of such decreased activity or expression include the following: Activity of the protein or expression of the gene encoding the protein is decreased below the level of that in wild-type, non-transgenic controls.

An “expression cassette” refers to a nucleic acid construct, which when introduced into a host cell, results in transcription and/or translation of a RNA or polypeptide, respectively. Expression cassettes can be derived from a variety of sources depending on the host cell to be used for expression. For example, an expression cassette can contain components derived from a viral, bacterial, insect, or mammalian source. In the case of both expression of transgenes and inhibition of endogenous genes (e.g., by antisense, or sense suppression) one of skill will recognize that the inserted polynucleotide sequence need not be identical and can be “substantially identical” to a sequence of the gene from which it was derived. As explained below, these variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “polynucleotide sequence from” a particular gene. In addition, the term specifically includes sequences (e.g., full length sequences) substantially identical with a gene sequence encoding a polypeptide of the present invention, e.g., SEQ ID NO: 3, 5, 14, or 17 and that encode proteins that retain the function of a protein of the present invention, e.g., specific binding to PEDF.

In the case of polynucleotides used to inhibit expression of an endogenous gene, the introduced sequence need not be perfectly identical to a sequence of the target endogenous gene. The introduced polynucleotide sequence will typically be at least substantially identical to the target endogenous sequence.

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity is evaluated using any of the variety of sequence comparison algorithms and programs known in the art. Such algorithms and programs include, but are by no means limited to, TBLASTN, BLASTP, FASTA, TFASTA, CLUSTALW, FASTDB, the disclosures of which are incorporated by reference in their entireties. Pearson, et al., Proc. Natl. Acad. Sci. U.S.A., 85: 2444-2448, 1988; Altschul, et al., J. Mol. Biol., 215: 403-410, 1990; Thompson, et al., Nucleic Acids Res., 22: 4673-4680, 1994; Higgins, et al., Meth. Enzymol., 266: 383402, 1996; Altschul, et al., Nature Genetics, 3: 266-272, 1993; Brutlag, et al., Comp. App. Biosci., 6: 237-24, 1990.

In a particularly preferred embodiment, protein and nucleic acid sequence identities are evaluated using the Basic Local Alignment Search Tool (“BLAST”) which is well known in the art the disclosures of which are incorporated by reference in their entireties. In particular, five specific BLAST programs are used to perform the following task: (1) LASTP and BLAST3 compare an amino acid query sequence against a protein sequence database; (2) BLASTN compares a nucleotide query sequence against a nucleotide sequence database; (3) LASTX compares the six-frame conceptual translation products of a query nucleotide sequence (both strands) against a protein sequence database; (4) BLASTN compares a query protein sequence against a nucleotide sequence database translated in all six reading frames (both strands); and (5) BLASTX compares the six-frame translations of a nucleotide query sequence against the six-frame translations of a nucleotide sequence database. Karlin, et al., Proc. Natl. Acad. Sci. U.S.A., 87: 2267-2268, 1990; Altschul, et al., Nuc. Acids Res., 25: 3389-3402, 1997.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix, the disclosures of which are incorporated by reference in their entireties). Less preferably, the PAM or PAM250 matrices can also be used (see, e.g., Schwartz, et al., eds., Matrices For Detecting Distance Relationships: Atlas Of Protein Sequence And Structure, Washington: National Biomedical Research Foundation, 1978, the disclosure of which is incorporated by reference in its entirety). The BLAST programs evaluate the statistical significance of all high-scoring segment pairs identified, and preferably select those segments which satisfy a user-specified threshold of significance, such as a user-specified percent homology. Preferably, the statistical significance of a high-scoring segment pair is evaluated using the statistical significance formula of Karlin, the disclosure of which is incorporated by reference in its entirety. The BLAST programs can be used with the default parameters or with modified parameters provided by the user. Gonnet, et al., Science, 256: 1443-1445, 1992; Henikoff, et al., Proteins, 17: 49-61, 1993; Karlin, et al., 1990.

Another preferred method for determining the best overall match between a query nucleotide sequence (a sequence of the present invention) and a subject sequence, also referred to as a global sequence alignment, can be determined using the FASTDB computer program based on the algorithm of Brutlag, et al. 1990, the disclosure of which is incorporated by reference in its entirety. In a sequence alignment the query and subject sequences are both DNA sequences. An RNA sequence can be compared by first converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty-0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using 10, the specified parameters, to arrive at a final percent identity score. This corrected score is what is used for the purposes of the present invention. Only nucleotides outside the 5′ and 3′ nucleotides of the subject sequence, as displayed by the FASTDB alignment, which are not matched/aligned with the query sequence, are calculated for the purposes of manually adjusting the percent identity score. For example, a 90 nucleotide subject sequence is aligned to a 100 nucleotide query sequence to determine percent identity. The deletions occur at the 5′ end of the subject sequence and therefore, the FASTDB alignment does not show a matched/alignment of the first 10 nucleotides at 5′ end. The 10 unpaired nucleotides represent 10% of the sequence (number of nucleotides at the 5′ and 3′ ends not matched/total number of nucleotides in the query sequence) so 10% is subtracted from the percent identity score calculated by the FASTDB program. If the remaining 90 nucleotides were perfectly matched the final percent identity would be 90%. In another example, a 90 nucleotide subject sequence is compared with a 100 nucleotide query sequence. This time the deletions are internal deletions so that there are no nucleotides on the 5′ or 3′ of the subject sequence which are not matched/aligned with the query. In this case the percent identity calculated by FASTDB is not manually corrected. Once again, only nucleotides 5′ and 3′ of the subject sequence which are not matched/aligned with the query sequence are manually corrected. No other manual corrections are made for the purposes of the present invention.

The nucleic acid compositions of the present invention, while often in a native sequence (except for modified restriction sites and the like), from either cDNA, genomic or mixtures may be mutated, thereof in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

To improve or alter the characteristics of polypeptides of the present invention, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the polypeptides of the present invention can be produced as multimers including dimers, trimers and tetramers. Multimerization can be facilitated by linkers or recombinantly though heterologous polypeptides such as Fc regions.

It is known in the art that one or more amino acids can be deleted from the N-terminus or C-terminus without substantial loss of biological function. See, e.g., Ron, et al., Biol Chem., 268: 2984-2988, 1993. Accordingly, the present invention provides polypeptides having one or more residues deleted from the amino terminus. Similarly, many examples of biologically functional C-terminal deletion mutants are known (see, e.g., Dobeli, et al., 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides which show substantial PEDF-R polypeptide activity. Such mutants include deletions, insertions, inversions, repeats, and substitutions selected according to general rules known in the art so as to have little effect on activity.

There are two main approaches for studying the tolerance of an amino acid sequence to change, see, Bowie, et al., Science, 247: 1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the ramide residues Asn and Gln, exchange of the basic residues Lys and Arg and replacements among the aromatic residues Phe, Tyr. Thus, the polypeptide of the present invention can be, for example: (i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the PEDF-R polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the polypeptides of the present invention can include one or more amino acid substitutions, deletions, or additions, either from natural mutations or human manipulation. As indicated, changes are preferably of a minor nature, such as conservative amino acid substitutions that do not significantly affect the folding or activity of the protein. The following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Furthermore, polypeptides of the present invention can include one or more amino acid substitutions that mimic modified amino acids. An example of this type of substitution includes replacing amino acids that are capable of being phosphorylated (e.g., serine, threonine, or tyrosine) with a negatively charged amino acid that resembles the negative charge of the phosphorylated amino acid (e.g., aspartic acid or glutamic acid). Also included is substitution of amino acids that are capable of being modified by hydrophobic groups (e.g., arginine) with amino acids carrying bulky hydrophobic side chains, such as tryptophan or phenylalanine. Therefore, a specific embodiment of the invention includes PEDF-R polypeptides that include one or more amino acid substitutions that mimic modified amino acids at positions where amino acids that are capable of being modified are normally positioned. Further included are PEDF-R polypeptides where any subset of modifiable amino acids is substituted. For example, a PEDF-R polypeptide that includes three serine residues can be substituted at any one, any two, or all three of said serines. Furthermore, any PEDF-R polypeptide amino acid capable of being modified can be excluded from substitution with a modification-mimicking amino acid.

The present invention is further directed to fragments of the polypeptides of the present invention. More specifically, the present invention embodies purified, isolated, and recombinant polypeptides comprising at least any one integer between 6 and 504 (or the length of the polypeptides amino acid residues minus 1 if the length is less than 1000) of consecutive amino acid residues. Preferably, the fragments are at least 6, preferably at least 8 to 10, more preferably 12, 15, 20, 25, 30, 35, 40, 50, 60, 75, 100, 125, 150, 175, 200, 225, 250, 275, 300, 325, 350, 360, or more consecutive amino acids of a polypeptide of the present invention.

The present invention also provides for the exclusion of any species of polypeptide fragments of the present invention specified by 5′ and 3′ positions or sub-genuses of polypeptides specified by size in amino acids as described above. Any number of fragments specified by 5′ and 3′ positions or by size in amino acids, as described above, can be excluded.

A preferred embodiment of the present invention is directed to epitope-bearing polypeptides and epitope-bearing polypeptide fragments. These epitopes can be “antigenic epitopes” or both an “antigenic epitope” and an “immunogenic epitope”. An “immunogenic epitope” is defined as a part of a protein that elicits an antibody response in vivo when the polypeptide is the immunogen. On the other hand, a region of polypeptide to which an antibody binds is defined as an “antigenic determinant” or “antigenic epitope.” The number of immunogenic epitopes of a protein generally is less than the number of antigenic epitopes (see, e.g., Geysen, et al., Proc. Natl. Acad. Sci. U.S.A., 81: 3998-4002, 1984, which disclosure is hereby incorporated by reference in its entirety). It is particularly noted that although a particular epitope cannot be immunogenic, it is nonetheless useful since antibodies can be made to both immunogenic and antigenic epitopes. When the antigen is a polypeptide, it is customary to classify epitopes as being linear (i.e., composed of a contiguous sequence of amino acids repeated along the polypeptide chain) or nonlinear (i.e., composed of amino acids brought into proximity as a result of the folding of the polypeptide chain). Nonlinear epitopes are also called “conformational” because they arise through the folding of the polypeptide chain into a particular conformation, i.e., a distinctive 3-D shape.

An epitope can comprise as few as 3 amino acids in a spatial conformation, which is unique to the epitope. Generally an epitope consists of at least 6 such amino acids, and more often at least 8-10 such amino acids. In preferred embodiment, antigenic epitopes comprise a number of amino acids that is any integer between 3 and 50. Fragments which function as epitopes can be produced by any conventional means (see, e.g., Houghten, Proc. Natl. Acad. Sci. U.S.A., 82: 5131-5135, 1985), also further described in U.S. Pat. No. 4,631,21, which disclosures are hereby incorporated by reference in their entireties. Methods for determining the amino acids which make up an epitope include x-ray crystallography, 2-dimensional nuclear magnetic resonance, and epitope mapping, e.g. the Pepscan method described by Geysen, et al., 1984; PCT Publication No. WO 84/03564; and PCT Publication No. WO 84/03506, which disclosures are hereby incorporated by reference in their entireties. Nonlinear epitopes are determined by methods such as protein footprinting (U.S. Pat. No. 5,691,448, which disclosure is hereby incorporated by reference in its entirety). Another example is the algorithm of Jameson, et al., Comp. Appl. Biosci., 4: 181-186, 1988, (said reference incorporated by reference in its entirety). The Jameson-Wolf antigenic analysis, for example, can be performed using the computer program PROTEAN, using default parameters (Version 4.0 Windows, DNASTAR, Inc., 1228 South Park Street Madison, Wis.)

Preferably, the epitope-containing polypeptide comprises a contiguous span of at least 6, preferably at least 8 to 10, more preferably 12, 15, 20, 25, 30, 35, 40, 50, 60, 75, 100, 125, 150, 175, 200, 225, 250, 275, or 300 or more amino acids of a polypeptide of the present invention.

Nonlinear epitopes comprise more than one noncontiguous polypeptide sequence of at least one amino acid each. Such epitopes result from noncontiguous polypeptides brought into proximity by secondary, tertiary, or quaternary structural features. Therefore, the present invention encompasses isolated, purified, or recombinant polypeptides and fragments thereof which comprise a nonlinear epitope. Preferred polypeptides providing nonlinear epitopes are formed by a contiguous surface of natively folded protein and are thus at least 10 amino acids in length, further preferably 12, 15, 20, 25, 30, 35, 40, 50, 60; 75, 100, 125, 150, 175, 200, 225, 250, 275, 300 or more amino acids of a polypeptide of the present invention, to the extent that a contiguous span of these lengths is consistent with the lengths of said selected sequence. Further preferred polypeptides comprise full-length polypeptide sequences selected from the group consisting of the polypeptide sequences of the Sequence Listing. Additionally, nonlinear epitopes can be formed by synthetic peptides that mimic an antigenic site or contiguous surface normally presented on a protein in the native conformation. Therefore, preferred polypeptides providing nonlinear epitopes can be formed by synthetic proteins that comprise a combination of at least 5, 6, 7, 8, 9, 10, 12, 15, 20, 25, 30, 35, 40, 50, 60, 75, 100, 125, 150, 175, 200, 225, 250, 275, or 300 or amino acids.

The epitope-bearing fragments of the present invention can comprise 6 to 50 amino acids (i.e. any integer between 6 and 50, inclusive) of a polypeptide of the present invention. Also, included in the present invention are antigenic fragments between the integers of 6 and the full length PEDF-R sequence of the sequence listing. All combinations of sequences between the integers of 6 and the full-length sequence of a PEDF-R polypeptide are included. The epitope-bearing fragments can be specified by either the number of contiguous amino acid residues (as a sub-genus) or by specific N-terminal and C-terminal positions (as species) as described above for the polypeptide fragments of the present invention. Any number of epitope-bearing fragments of the present invention can also be excluded in the same manner.

Antigenic epitopes are useful, for example, to raise antibodies, including monoclonal antibodies that specifically bind the epitope (see, Wilson, et al., 1984; Sutcliffe, et al., Science, 219: 660-666, 1983, which disclosures are hereby incorporated by reference in their entireties). The antibodies are then used in various techniques such as diagnostic and tissue/cell identification techniques, as described herein, and in purification methods such as immunoaffinity chromatography.

As one of skill in the art will appreciate, and discussed above, the polypeptides of the present invention optionally comprising an immunogenic or antigenic epitope can be fused to heterologous polypeptide sequences. For example, the polypeptides of the present invention can be fused with the constant domain of immunoglobulins (IgA, IgE, IgG, IgM), or portions thereof (CH1, CH2, CH3, any combination thereof including both entire domains and portions thereof) resulting in chimeric polypeptides. These fusion proteins facilitate purification, and show an increased half-life in vivo. This has been shown, e.g., for chimeric proteins consisting of the first two domains of the human CD4-polypeptide and various domains of the constant regions of the heavy or light chains of mammalian immunoglobulins (see, e.g., EPA 0,394,827; Traunecker, et al., Nature, 331: 84-86, 1988, which disclosures are hereby incorporated by reference in their entireties). Fusion proteins that have a disulfide-linked dimeric structure due to the IgG portion can also be more efficient in binding and neutralizing, other molecules than monomeric polypeptides or fragments thereof alone (see, e.g., Fountoulakis, et al., Biochem., 270: 3958-30 3964, 1995, which disclosure is hereby incorporated by reference in its entirety). Nucleic acids encoding the above epitopes can also be recombined with a gene of interest as an epitope tag to aid in detection and purification of the expressed polypeptide.

Additional fusion proteins of the invention can be generated through the techniques of gene-shuffling, motif-shuffling, exon-shuffling, or codon-shuffling (collectively referred to as “DNA shuffling”). DNA shuffling can be employed to modulate the activities of polypeptides of the present invention thereby effectively generating agonists and antagonists of the polypeptides. See, for example, U.S. Pat. Nos. 5,605,793; 5,811,238; 5,834,252; 5,837,458; Patten, et al., Curr. Opinion Biotechnol., 8: 724-733, 1997; Harayama, Trends Biotechnol., 16: 76-82, 1998; Hansson, et al., J. Mol. Biol., 287: 265-276, 1999; Lorenzo, et al., Biotechniques, 24: 308-313, 1998. (Each of these documents is hereby incorporated by reference). In one embodiment, one or more components, motifs, sections, parts, domains, fragments, etc., of coding polynucleotides of the invention, or the polypeptides encoded thereby can be recombined with one or more components, motifs, sections, parts, domains, fragments, etc. of one or more heterologous molecules.

The “PEDF-R extracellular domain” is a form of the PEDF-R which is essentially free of the transmembrane and cytoplasmic domains of PEDF-R, i.e., has less than 1% of such domains, preferably 0.5 to 0% of such domains, and more preferably 0.1 to 0% of such domains. For example, SEQ ID NO:5 is part of the PEDF-R extracellular domain.

A “chimeric PEDF-R” is a polypeptide comprising full-length PEDF-R or one or more domains thereof (e.g., the extracellular domain) fused or bonded to heterologous polypeptide. The chimeric PEDF-R will generally share at least one biological property in common with PEDF-R. Examples of chimeric PEDF-R include immunoadhesins and epitope-tagged PEDF-R

The term “immunoadhesin” is used interchangeably with the expression “PEDF-R immunoglobulin chimera” and refers to a chimeric molecule that combines a portion of the PEDF-R (generally the extracellular domain thereof) with an immunoglobulin sequence. The immunoglobulin sequence preferably, but not necessarily, is an immunoglobulin constant domain. The immunoglobulin moiety in the chimeras of the present invention can be obtained from IgG1, IgG2, IgG3 or IgG4 subtypes, IgA, IgE, IgD or IgM, but preferably IgG1 or IgG3.

The term “epitope-tagged” when used herein refers to a chimeric polypeptide comprising PEDF-R fused to a “tag polypeptide”. The tag polypeptide has enough residues to provide an epitope against which an antibody can be made, yet is short enough such that it does not interfere with biological activity of the PEDF-R. The tag polypeptide preferably also is fairly unique so that the antibody does not substantially cross-react with other epitopes. Suitable tag polypeptides generally have at least six amino acid residues and usually between about 8-50 amino acid residues (preferably between about 9-30 residues). Preferred are poly-histidine sequences, which bind nickel, allowing isolation of the tagged protein by Ni-NTA chromatography as described for example in Lindsay, et al. Neuron, 17: 571-574, 1996.

The nucleic acids of the invention are present in whole cells, in a cell lysate, or in a partially purified or substantially pure form. A nucleic acid is “isolated” when purified away from other cellular components or other contaminants, e.g., other cellular nucleic acids or proteins, by standard techniques, including alkaline/SDS treatment, CsCl banding, column chromatography, agarose gel electrophoresis and others well known in the art (See, e.g., Sambrook, Tijssen and Ausubel discussed herein and incorporated by reference for all purposes). The nucleic acid sequences of the invention and other nucleic acids used to practice this invention, whether RNA, cDNA, genomic DNA, or hybrids thereof, may be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook, Tijssen and Ausubel. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

“Isolated PEDF-R” means PEDF-R that has been purified from a PEDF-R source, e.g., retinal cells, or has been prepared by recombinant or synthetic methods and is sufficiently free of other peptides or proteins.

“Essentially pure” protein means a composition comprising at least about 90% by weight of the protein, based on total weight of the composition, preferably at least about 95% by weight. “Essentially homogeneous” protein means a composition comprising at least about 99% by weight of protein, based on total weight of the composition.

“Inhibitors,” “activators,” and “modulators” of PEDF-R activity are used to refer to inhibitory, activating, or modulating molecules, respectively, identified using in vitro and in vivo assays for PEDF-R binding or signaling, e.g., ligands, agonists, antagonists, and their homologs and mimetics.

The term “modulator” includes inhibitors and activators. Inhibitors are agents that, e.g., bind to, partially or totally block stimulation, decrease, prevent, delay activation, inactivate, desensitize, or down regulate the activity of PEDF-Rs, e.g., antagonists. Activators are agents that, e.g., bind to, stimulate, increase, open, activate, facilitate, enhance activation, sensitize or up regulate the activity of PEDF-Rs, e.g., agonists. Modulators include agents that, e.g., alter the interaction of PEDF-Rs with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring PEDF-R ligands, e.g., with altered activity, as well as naturally occurring and synthetic ligands, antagonists, agonists, small chemical molecules and the like. Such assays for inhibitors and activators include, e.g., applying putative modulator compounds to a cell expressing a PEDF-R and then determining the functional effects on PEDF-R signaling, as described herein. Samples or assays comprising PEDF-R that are treated with a potential activator, inhibitor, or modulator are compared to control samples without the inhibitor, activator, or modulator to examine the extent of inhibition. Control samples (untreated with inhibitors) can be assigned a relative PEDF-R activity value of 100%. Inhibition of PEDF-R is achieved, for example, when the PEDF-R activity value relative to the control is about 80%, optionally 50% or 25-0%. Activation of PEDF-R is achieved when the PEDF-R activity value relative to the control is 110%, optionally 150%, optionally 200-500%, or 1000-3000% higher. Exemplary PEDF-R binding activity assays of the present invention are: a PEDF ligand blot assay (Aymerich, et al., Invest Ophthalmol. Vis. Sci., 42: 3287-93, 2001); a PEDF affinity column chromatography assay (Alberdi, J. Biol. Chem., 274: 31605-12, 1999) and a PEDF Ligand binding assay (Alberdi, et al., J. Biol. Chem., 274: 31605-12, 1999). These references are herein incorporated by reference for all purposes.

The ability of a molecule to bind to PEDF-R can be determined, for example, by the ability of the putative ligand to bind to PEDF-R immunoadhesin coated on an assay plate. Specificity of binding can be determined by comparing binding to PEDF-R.

PEDF binding assays, for example, include radioligand binding assays for PEDF to cells, plasma membranes, detergent-solubilized plasma membrane proteins, immobilized collagen (Alberdi et al., 1999, JBC; Meyer et al., 2002); PEDF-affinity column chromatography (Alberdi, et al., J. B. C., 1999; Aymerich, et al., 2001); PEDF ligand blot using a radio- or fluorosceinated-ligand (Aymerich, et al., 2001; Meyer, et al., 2002); Size-exclusion ultrafiltration (Alberdi, et al., Biochem., 1998; Meyer, et al., 2002); or ELISA.

In one embodiment, PEDF binding to PEDF-R can be assayed by either immobilizing the ligand or the receptor. For example, the assay can include immobilizing PEDF-R fused to a His tag onto Ni-activated NTA resin beads. PEDF can be added in an appropriate buffer and the beads incubated for a period of time at a given temperature. After washes to remove unbound material, the bound protein can be released with, for example, SDS, buffers with a high pH, and the like and analyzed.

PEDF binding to PEDF-R has been identified, for example, in the inner segments of photoreceptors and the retinal ganglion cell layer of bovine retina sections, on the surface of human retinoblastoma Y-79 cells, cerebellar granule cell neurons, and motor neurons, and on the surface of endothelias cells, HUVECs and BRECs.

The terms “biological activity” and “biologically active” with regard to a PEDF ligand of the present invention refer to the ability of a molecule to specifically bind to and signal through a native or recombinant PEDF-R, or to block the ability of a native or recombinant PEDF-R to participate in signal transduction. Thus, the (native and variant) PEDF ligands of the present invention include agonists and antagonists of a native or recombinant PEDF-R. Preferred biological activities of the PEDF ligands of the present invention include the ability to induce or inhibit, for example, neovascularization, neurotrophic activity, neuronotrophic activity, angiogenic activity, gliastatic activity, or obesity related disorders. The ability to induce vascularization will be useful for the treatment of biological conditions and diseases, where vascularization is desirable, such as wound healing. On the other hand, the ability to inhibit or block vascularization may, for example, be useful in preventing cell proliferative disorders and other diseases where neovascularization is not desirable, for example, cancer, ischaemia, and diabetic retinopathy. The ability of PEDF to prevent the accumulation of lipids or bring about the dissipation of the accumulation of lipids is also desirable. In several retinal pathologies (e.g., AMD, Diabetic Retinopathy) RPE cells have been shown to produce lipid accumulations, and in the same pathologies cell death and angiogenesis accompany this accumulation. PEDF, as anti-angiogenic and neurotrophic factor prevent angiogenesis and neuronal cell death respectively. As disclosed herein, PEDF interacts with a membrane receptor (R1) and this interaction brings about phospholipasic activity (others have shown a generic trigliceride lipase activity is possible can also be brought about by PEDF). For its particular interaction with R1, PEDF could activate R1 's lipasic activity, decreasing the lipid accumulation (in the same way it decreases lipid droplets in the differentiated NIH3T3-L1 cells), protecting neuronal cells and photoreceptors from apoptotic death and/or preventing angiogenesis.

The term “high affinity” for a ligand refers to an equilibrium association constant (Ka) of at least about 10³M⁻¹, at least about 10⁴M⁻¹, at least about 10⁵M⁻¹, at least about 10⁶M⁻¹, at least about 10⁷M⁻¹, at least about 10⁸M⁻¹, at least about 10⁹M⁻¹, at least about 10¹⁰M⁻¹, at least about 10¹¹M⁻¹, or at least about 10¹²M⁻¹ or greater, e.g., up to 10¹³M⁻¹ or 10¹⁴M⁻¹ or greater. However, “high affinity” binding can vary for other ligands.

The term “Kₐ”, as used herein, is intended to refer to the equilibrium association constant of a particular ligand-receptor interaction, e.g., antibody-antigen interaction. This constant has units of 1/M.

The term “K_{d}”, as used herein, is intended to refer to the equilibrium dissociation constant of a particular ligand-receptor interaction. This constant has units of M.

The term “kₐ”, as used herein, is intended to refer to the kinetic association constant of a particular ligand-receptor interaction. This constant has units of 1/s.

The term “k_{d}”, as used herein, is intended to refer to the kinetic dissociation constant of a particular ligand-receptor interaction. This constant has units of 1/s.

“Particular ligand-receptor interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

The term “antagonist” is used in the broadest sense, and includes any molecule that partially or fully blocks, inhibits, or neutralizes a biological activity of a PEDF-R polypeptide. In a similar manner, the term “agonist” is used in the broadest sense and includes any molecule that mimics or enhances a biological activity of a PEDF-R polypeptide. Suitable agonist or antagonist molecules specifically include agonist or antagonist antibodies or antibody fragments, fragments or amino acid sequence variants of native PEDF polypeptides, peptides, antisense oligonucleotides, small organic molecules, and the like. Methods for identifying agonists or antagonists of a PEDF-R polypeptide can comprise contacting a PEDF-R polypeptide with a candidate agonist or antagonist molecule and measuring a detectable change in one or more biological activities normally associated with the PEDF-R polypeptide.

The expression “control sequences” or “regulatory sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors, are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

The term “sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny cannot be precisely identical in DNA content, due to deliberate or inadvertent mutations. Mutant progeny that have the same function or biological activity as screened for in the originally transformed cell are included. Where distinct designations are intended, it will be clear from the context.

The term “receptor” denotes a cell-associated protein, for example PEDF-R that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors, for example PEDF-R, are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

The phrase “enhancing proliferation of a cell” encompasses the step of increasing the extent of growth and/or reproduction of the cell relative to an untreated cell either in vitro or in vivo. An increase in cell proliferation in cell culture can be detected by counting the number of cells before and after exposure to a molecule of interest. The extent of proliferation can be quantified via microscopic examination of the degree of confluence.

By “enhancing differentiation of a cell” is meant the act of increasing the extent of the acquisition or possession of one or more characteristics or functions which differ from that of the original cell (i.e. cell specialization). This can be detected by screening for a change in the phenotype of the cell (e.g., identifying morphological changes in the cell).

The term “treating” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations. Accordingly, the term “treating” includes the administration of the compounds or agents of the present invention to inhibit tumor angiogenesis, tumor growth, or to cause the regression of already existing tumors. It also includes the administration of the compounds of the present invention to promote neurotrophic, neurotronophic, or gliastatic activity in a subject. Accordingly, the term “treating” includes the administration of the compounds or agents of the present invention to prevent or delay, to alleviate, or to arrest or inhibit development of the symptoms or conditions associated with angiogenesis, ocular disease, neural diseases or disorders, and obesity-related disorders. The term “therapeutic effect” refers to the reduction, elimination, or prevention of the disease, symptoms of the disease, or side effects of the disease in the subject.

“Concomitant administration” of a known drug with a compound of the present invention means administration of the drug and the compound at such time that both the known drug and the compound will have a therapeutic effect or diagnostic effect. Such concomitant administration can involve concurrent (i.e. at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” is understood as a cell having specific structural properties, lacking differentiation and being capable of invasion and metastasis. Examples of cancers are kidney, colon, breast, prostate and liver cancer. (see DeVita, et al. (eds.), Cancer Principles and Practice of Oncology, 6th. Ed., Lippincott Williams & Wilkins, Philadelphia, Pa., 2001; this reference is herein incorporated by reference in its entirety for all purposes).

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

In the context of the cancer, the term “transformation” refers to the change that a normal cell undergoes as it becomes malignant. In eukaryotes, the term “transformation” can be used to describe the conversion of normal cells to malignant cells in cell culture.

“Proliferating cells” are those which are actively undergoing cell division and growing exponentially. “Loss of cell proliferation control” refers to the property of cells that have lost the cell cycle controls that normally ensure appropriate restriction of cell division. Cells that have lost such controls proliferate at a faster than normal rate, without stimulatory signals, and do not respond to inhibitory signals.

The term “apoptosis” and “programmed cell death” (PCD) are used as synonymous terms and describe the molecular and morphological processes leading to controlled cellular self-destruction (see, e.g., Kerr, et al., Br. J. Cancer., 26: 239-257, 1972). Apoptotic cell death can be induced by a variety of stimuli, such as ligation of cell surface receptors, starvation, growth factor/survival factor deprivation, heat shock, hypoxia, DNA damage, viral infection, and cytotoxic/chemotherapeutical agents. The apoptotic process is involved in embryogenesis, differentiation, proliferation/homoeostasis, removal of defect and therefore harmful cells, and especially in the regulation and function of the immune system. Thus, dysfunction or disregulation of the apoptotic program is implicated in a variety of pathological conditions, such as immunodeficiency, autoimmune diseases, neurodegenerative diseases, and cancer. Apoptotic cells can be recognized by stereotypical morphological changes: the cell shrinks, shows deformation and looses contact to its neighboring cells. Its chromatin condenses, and finally the cell is fragmented into compact membrane-enclosed structures, called “apoptotic bodies” which contain cytosol, the condensed chromatin, and organelles. The apoptotic bodies are engulfed by macrophages and thus are removed from the tissue without causing an inflammatory response. This is in contrast to the necrotic mode of cell death in which case the cells suffer a major insult, resulting in loss of membrane integrity, swelling and disrupture of the cells. During necrosis, the cell contents are released uncontrolled into the cell's environment what results in damage of surrounding cells and a strong inflammatory response in the corresponding tissue. See, e.g., Tomei, et al., (eds.), APOPTOSIS: THE MOLECULAR BASIS OF CELL DEATH, PLAINVILLE, NY: Cold Spring Harbor Laboratory Press, 1991; Isaacs, et al., Environ. Health. Perspect., 101: 27-33, 1993; each of which is herein incorporated by reference in its entirety for all purposes. A variety of apoptosis assays are well known to one of skill in the art (e.g., DNA fragmentation assays, radioactive proliferation assays, DNA laddering assays for treated cells, Fluorescence microscopy of 4′-6-Diamidino-2-phenylindole (DAPI) stained cells assays, and the like).

The term “subject” or “patient” as used herein means any mammalian patient or subject to which the compositions of the invention can be administered. The term mammals, human patients and non-human primates, as well as experimental animals such as rabbits, rats, and mice, and other animals. In an exemplary embodiment, of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other routine methods allow the clinician to select patients in need of therapy using the methods and formulations of the invention.

By “solid phase” is meant a non-aqueous matrix to which a reagent of interest (e.g., the PEDF-R or an antibody thereto) can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

“Neurotrophic” activity is defined herein as the ability to induce differentiation of a neuronal cell population. For example, PEDF's ability to induce differentiation in cultured retinoblastoma cells is considered neurotrophic activity.

“Neuronotrophic” activity is defined herein as the ability to enhance survival of neuronal cell populations. For example, PEDF's ability to act as a neuron survival factor on neuronal cells is neuronotrophic activity.

“Gliastatic” activity is defined herein as the ability to inhibit glial cell growth and proliferation. For example, PEDF's ability to prevent growth and/or proliferation of glial cells is gliastatic activity.

“Adipostatic” activity is defined herein as the as the ability to modulate adipocyte differentiation. For example, PEDF's ability to block adipogenesis of preadiocytes NIH-3T3-L1 cells is adipostatic activity.

The phrase “specifically (or selectively) binds” to an antibody refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

The phrase “specifically bind(s)” or “bind(s) specifically” when referring to a peptide refers to a peptide molecule which has intermediate or high binding affinity, exclusively or predominately, to a target molecule. The phrase “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify peptides that specifically react with PEDF domain-containing proteins. Typically a specific or selective reaction will be at least twice background signal or noise and more typically more than 10 times background. Specific binding between PEDF and PEDF-R means a binding affinity of at least 10³ M⁻¹, and preferably 10⁵, 10⁶, 10⁷, 10⁸, 10⁹ or 10¹⁰ M⁻¹. The binding affinity of PEDF to PEDF-R is preferably between about 10⁶ M⁻¹ to about 10¹⁰ M⁻¹.

The present invention is based on the discovery of the PEDF-R, a protein that binds PEDF with a high affinity. The experiments described herein demonstrate that this molecule is a receptor which plays a role in mediating responses to PEDF. In particular, this receptor has been found to be present in a variety of tissue and cell populations, including neurons, thus indicating that PEDF ligands, such as agonist antibodies, can be used to stimulate proliferation, growth, survival, differentiation, metabolism, or regeneration of PEDF-R containing cells.

This invention relies on routine techniques in the field of recombinant genetics. Basic texts disclosing the general methods of use in this invention include Sambrook, et al., Molecular Cloning, A Laboratory Manual, 2nd ed., 1989; Kriegler, Gene Transfer and Expression: A Laboratory Manual, 1990; Ausubel, et al., (eds.), Current Protocols in Molecular Biology, 1994.

PEDF-R nucleic acids, polymorphic variants, orthologs, and alleles that are substantially identical to sequences provided herein can be isolated using PEDF-R nucleic acid probes and oligonucleotides under stringent hybridization conditions, by screening libraries. Alternatively, expression libraries can be used to clone PEDF-R protein, polymorphic variants, orthologs, and alleles by detecting expressed homologs immunologically with antisera or purified antibodies made against human PEDF-R or portions thereof.

To make a cDNA library, one should choose a source that is rich in PEDF-R RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler, et al., Gene, 25: 263-269, 1983; Sambrook, et al., supra, 1983; Ausubel, et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton, et al., Science, 196: 180-182, 1977. Colony hybridization is carried out as generally described in Grunstein, et al., Proc. Natl. Acad. Sci. U.S.A., 72: 3961-3965, 1975.

An alternative method of isolating PEDF-R nucleic acid and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; Innis, et al., PCR Protocols: A Guide to Methods and Applications, eds. 1990). Methods such as polymerase chain reaction (PCR) and ligase chain reaction (LCR) can be used to amplify nucleic acid sequences of human PEDF-R directly from mRNA, from cDNA, from genomic libraries or cDNA libraries. Degenerate oligonucleotides can be designed to amplify PEDF-R homologs using the sequences provided herein. Restriction endonuclease sites can be incorporated into the primers. Polymerase chain reaction or other in vitro amplification methods can also be useful, for example, to clone nucleic acid sequences that code for proteins to be expressed, to make nucleic acids to use as probes for detecting the presence of PEDF-R encoding mRNA in physiological samples, for nucleic acid sequencing, or for other purposes. Genes amplified by the PCR reaction can be purified from agarose gels and cloned into an appropriate vector.

Gene expression of PEDF-R can also be analyzed by techniques known in the art, e.g., reverse transcription and amplification of mRNA, isolation of total RNA or poly A⁺ RNA, northern blotting, dot blotting, in situ hybridization, RNase protection, high density polynucleotide array technology, e.g., and the like.

Nucleic acids encoding PEDF-R protein can be used with high density oligonucleotide array technology (e.g., GeneChip™) to identify PEDF-R protein, orthologs, alleles, conservatively modified variants, and polymorphic variants in this invention. In the case where the homologs being identified are linked to PEDF related diseases, they can be used with GeneChip™ as a diagnostic tool in detecting the disease in a biological sample, see, e.g., Gunthand, et al., AIDS Res. Hum. Retroviruses, 14: 869-876, 1998; Kozal, et al., Nat. Med., 2: 753-759, 1996; Matson, et al., Anal. Biochem., 224: 110-106, 1995; Lockhart, et al., Nat. Biotechnol., 14: 1675-1680, 1996; Gingeras, et al., Genome Res., 8: 435-448, 1998; Hacia, et al., Nucleic Acids Res., 26: 3865-3866, 1998.

The gene for PEDF-R is typically cloned into intermediate vectors before transformation into prokaryotic or eukaryotic cells for replication and/or expression. These intermediate vectors are typically prokaryote vectors, e.g., plasmids, or shuttle vectors.

To obtain high level expression of a cloned gene, such as those cDNAs encoding PEDF-R, one typically subclones PEDF-R into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook, et al., Ausubel, et al., supra. Bacterial expression systems for expressing the PEDF-R protein are available in, e.g., E. coli, Bacillus sp., and Salmonella (Palva, et al., Gene, 22: 229-235, 1983; Mosbach, et al., Nature, 302: 543-545, 1983. Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. Promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to PEDF-R encoding DNA by removing the promoter from the source DNA by restriction enzyme digestion and inserting the isolated promoter sequence into the vector. Both the native PEDF-R promoter sequence and many heterologous promoters can be used to direct amplification and/or expression of the PEDF-R DNA. However, heterologous promoters are preferred, as they generally permit greater transcription and higher yields of PEDF-R as compared to the native PEDF-R promoter.

Promoters suitable for use with prokaryotic hosts include the β-lactamase and lactose promoter systems (Chang, et al., Nature, 275: 615, 1978; Goeddel, et al., Nature, 281: 544, 1979), alkaline phosphatase, a tryptophan (trp) promoter system (Goeddel, Nucleic Acids Res., 8: 4057, 1980; EP 36,776), and hybrid promoters such as the tac promoter. DeBoer, et al., Proc. Natl. Acad. Sci. U.S.A., 80: 21-25, 1983. However, other known bacterial promoters are suitable. Their nucleotide sequences have been published, thereby enabling a skilled worker operably to ligate them to DNA encoding PEDF-R (Siebenlist, et al., Cell, 20: 269, 1980) using linkers or adaptors to supply any required restriction sites. Promoters for use in bacterial systems also will contain a Shine-Delgamo (S. D.) sequence operably linked to the DNA encoding PEDF-R.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CXCAAT region where X can be any nucleotide. At the 3′ end of most eukaryotic genes is an AATAAA sequence that can be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase (Hitzeman, et al., J Biol. Chem., 255: 2073, 1980) or other glycolytic enzymes (Hess, et al., J Adv. Enzyme Reg., 7: 149, 1968; Holland, Biochemistry, 17: 4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

PEDF-R transcription from vectors in mammalian host cells is controlled, for example, by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, and from the promoter normally associated with the PEDF-R sequence, provided such promoters are compatible with the host cell systems. The early and late promoters of the SV40 virus are conveniently obtained as an SV40 restriction fragment that also contains the SV40 viral origin of replication. Fiers, et al., Nature, 273: 113, 1978; Mulligan, et al., Science, 209: 1422-1427, 1980; Pavlakis, et al., Proc. Natl. Acad. Sci. U.S.A., 78: 7398-7402, 1981. The immediate early promoter of the human cytomegalovirus is conveniently obtained as a HindIII E restriction fragment. Greenaway, et al., Gene, 18: 355-360, 1982. A system for expressing DNA in mammalian hosts using the bovine papilloma virus as a vector is disclosed in U.S. Pat. No. 4,419,446. A modification of this system is described in U.S. Pat. No. 4,601,978. See also Gray, et al., Nature, 295: 503-508, 1982, on expressing cDNA encoding immune interferon in monkey cells; Reyes, et al., Nature, 297: 598-601, 1982, on expression of human β-interferon cDNA in mouse cells under the control of a thymidine kinase promoter from herpes simplex virus; Canaani, et al., Proc. Natl. Acad. Sci. U.S.A., 79: 5166-5170, 1982, on expression of the human interferon β1 gene in cultured mouse and rabbit cells; and Gorman, et al., Proc. Natl. Acad. Sci. U.S.A., 79: 6777-6781, 1982, on expression of bacterial CAT sequences in CV-1 monkey kidney cells, chicken embryo fibroblasts, Chinese hamster ovary cells, HeLa cells, and mouse NIH-3T3 cells using the Rous sarcoma virus long terminal repeat as a promoter.

In addition to the promoter, the expression vector typically contains a transcription unit or expression cassette that contains all the additional elements required for the expression of the PEDF-R encoding nucleic acid in host cells. A typical expression cassette thus contains a promoter operably linked to the nucleic acid sequence encoding PEDF-R and signals required for efficient polyadenylation of the transcript, ribosome binding sites, and translation termination. Additional elements of the cassette can include enhancers and, if genomic DNA is used as the structural gene, introns with functional splice donor and acceptor sites.

In addition to a promoter sequence, the expression cassette can also contain a transcription termination region downstream of the structural gene to provide for efficient termination. The termination region can be obtained from the same gene as the promoter sequence or can be obtained from different genes.

The particular expression vector used to transport the genetic information into the cell is not particularly critical. Any of the conventional vectors used for expression in eukaryotic or prokaryotic cells can be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a PEDF-R encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of PEDF-R protein, which are then purified using standard techniques (see, e.g., Colley, et al, J. Biol. Chem., 264: 17619-17622, 1989; Deutscher, ed., Guide to Protein Purification, in Methods in Enzymology, vol. 182, 1990). Transformation of eukaryotic and prokaryotic cells are performed according to standard techniques (see, e.g., Morrison, Bact., 132: 349-351, 1977; Wu, et al., (eds.), Clark-Curtiss & Curtiss, Methods in Enzymology, 101: 347-362, 1983.

Any of the well-known procedures for introducing foreign nucleotide sequences into host cells can be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing PEDF-R.

After the expression vector is introduced into the cells, the transfected cells are cultured under conditions favoring expression of PEDF-R, which is recovered from the culture using standard techniques identified below.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. Strain W3110 is a particularly preferred host or parent host because it is a common host strain for recombinant DNA product fermentations. Preferably, the host cell should secrete minimal amounts of proteolytic enzymes. For example, strain W3110 can be modified to effect a genetic mutation in the genes encoding proteins, with examples of such hosts including E. coli W3110 strain 27C7. Alternatively, the strain of E. coli having mutant periplasmic protease disclosed in U.S. Pat. No. 4,946,783 issued Aug. 7, 1990 can be employed. Alternatively still, methods of cloning, e.g., PCR or other nucleic acid polymerase reactions, are suitable.

In addition to prokaryotes, eukaryotic microbes such as filamentous fungi or yeast are suitable cloning or expression hosts for PEDF-R-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe (Beach, et al., Nature, 290: 140, 1981; EP 139,383 published May 2, 1985); Kluyveromyces hosts (U.S. Pat. No. 4,943,529; Fleer, et al., supra) such as, e.g., K. lactis (MW98-8C, CBS683, CBS4574; Louvencourt, et al., J. Bacteriol., 737, 1983), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg, et al., supra), K. thermotolerans, and K. marxianus; yarrowia (EP 402,226); Pichia pastoris (EP 183,070; Sreekrishna, et al., J Basic Microbiol., 28: 265-278, 1988); Candida; Trichoderma reesia (EP 244,234); Neurospora crassa (Case, et al., Proc. Natl. Acad. Sci. U.S.A., 76: 5259-5263, 1979); Schwanniomyces such as Schwanniomyces occidentalis (EP 394,538 published Oct. 31, 1990); and filamentous fingi such as, e.g., Neurospora, Penicillium, Tolypocladium (WO 91/00357 published Jan. 10, 1991), and Aspergillus hosts such as A. nidulans (Balance, et al., Biochem. Biophys. Res. Commun., 112: 284-289, 1983; Tilburn, et al., Gene, 26: 205-221, 1983; Yelton, et al., Proc. Natl. Acad. Sci. U.S.A., 81: 1470-1474, 1984) and A. niger. (Kelly, et al., EMBO J., 4: 475-479, 1985).

Suitable host cells for the expression of glycosylated PEDF-R are derived from multicellular organisms. Such host cells are capable of complex processing and glycosylation activities. In principle, any higher eukaryotic cell culture is workable, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. See, e.g., Luckow, et al., Bio/Technology, 6: 47-55, 1988; Miller, et al., in GENETIC ENGINEERING, Setlow et al, eds., Vol. 8 (Plenum Publishing, 1986), pp. 277-279; Maeda, et al., Nature, 315: 592-594, 1985. A variety of viral strains for transfection are publicly available, e.g., the L-1 variant of Autographa californica NPV and the Bm-5 strain of Bombyx mori NPV, and such viruses can be used as the virus herein according to the present invention, particularly for transfection of Spodoptera frugiperda cells

Either naturally occurring or recombinant PEDF-R can be purified for use in functional assays. Naturally occurring PEDF-R can be purified, e.g., from human tissue. Recombinant PEDF-R can be purified from any suitable expression system.

The PEDF-R protein can be purified to substantial purity by standard techniques, including selective precipitation with such substances as ammonium sulfate; column chromatography, immunopurification methods, and others (see, e.g., Scopes; Protein Purification: Principles and Practice (1982); U.S. Pat. No. 4,673,641; Ausubel, et al., supra; Sambrook, et al., supra).

A number of procedures can be employed when recombinant PEDF-R protein is being purified. For example, proteins having established molecular adhesion properties can be reversible fused to the PEDF-R protein. With the appropriate ligand, PEDF-R protein can be selectively adsorbed to a purification column and then freed from the column in a relatively pure form. The fused protein is then removed by enzymatic activity. Finally, PEDF-R protein could be purified using immunoaffinity columns.

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

It is possible to purify PEDF-R protein from bacteria periplasm. After lysis of the bacteria, when the PEDF-R protein exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding an amount of ammonium sulfate to a known concentration to precipitate a protein of interest. For transmembrane proteins such as PEDF-R, saturated ammonium sulfate is added to the protein solution so that the resultant ammonium sulfate concentration is between 20-30%. This concentration will generally be sufficient to precipitate transmembrane proteins. If it is not sufficient, additional ammonium sulfate can be added until the protein is precipitated. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

In one embodiment, PEDF-R is purified by PEDF-affinity column chromatography (Alberdi, et al., 1999; Aymerich, et al., 2001; these references are herein incorporated by reference for all purposes). For example, the plasma membrane proteins can be isolated by differential centrifugation. These proteins can then be solubilized with detergents. The solubilized plasma membrane proteins containing the PEDF-R can then be subjected to PEDF-affinity column chromatography. The unbound proteins can be eluted. After several washes to remove the unbound proteins, 0.5 M NaCl can be added to remove proteins bound by ionic interactions with the column. To elute the PEDF-R, buffers at pH 2 is applied followed by buffers at pH 11. The PEDF-R can then be eluted with buffers at pH 11.

The molecular weight of the PEDF-R proteins can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The PEDF-R proteins can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Accordingly, the PEDF-R can be used for affinity purification of ligands that bind to the PEDF-R, either naturally-occurring or synthetic ligands. PEDF is a preferred ligand for purification. Briefly, this technique involves: (a) contacting a source of PEDF ligand with an immobilized PEDF-R under conditions whereby the PEDF ligand to be purified is selectively adsorbed onto the immobilized receptor; (b) washing the immobilized PEDF-R and its support to remove non-adsorbed material; and (c) eluting the PEDF ligand molecules from the immobilized PEDF-R to which they are adsorbed with an elution buffer. In a particularly preferred embodiment of affinity purification, PEDF-R is covalently attached to an inert and porous matrix or resin (e.g., agarose reacted with cyanogen bromide). Especially preferred is a PEDF-R immunoadhesin immobilized on a protein A column. A solution containing PEDF ligand is then passed through the chromatographic material. The PEDF ligand adsorbs to the column and is subsequently released by changing the elution conditions (e.g. by changing pH or ionic strength). Novel ligands can be detected by monitoring displacement of a known, labelled PEDF-R ligand, such as ¹²⁵I or biotinylated-PEDF.

In addition to the detection of PEDF-R genes and gene expression using nucleic acid hybridization technology, one can also use immunoassays to detect PEDF-R proteins of the invention. Such assays are useful for screening for modulators of PEDF-R regulation of cellular proliferation, as well as for therapeutic and diagnostic applications. Immunoassays can be used to qualitatively or quantitatively analyze PEDF-R proteins. A general overview of the applicable technology can be found in Harlow & Lane, Antibodies: A Laboratory Manual (1988). This reference is incorporated in its entirety for all purposes.

“Antibodies” as used herein includes polyclonal and monoclonal antibodies, chimeric, single chain, and humanized antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

The term “antibody” is used in the broadest sense and specifically covers monoclonal antibodies, antibody compositions with polyepitopic specificity, bispecific antibodies, diabodies, and single-chain molecules, as well as antibody fragments (e.g., Fab, F(ab′)₂, and Fv), so long as they exhibit the desired biological activity. Antibodies can be labeled/conjugated to toxic or non-toxic moieties. Toxic moieties include, for example, bacterial toxins, viral toxins, radioisotopes, and the like. Antibodies can be labeled for use in biological assays (e.g., radioisotope labels, fluorescent labels) to aid in detection of the antibody. Antibodies can also be labeled/conjugated for diagnostic or therapeutic purposes, e.g., with radioactive isotopes that deliver radiation directly to a desired site for applications such as radioimmunotherapy (Garmestani, et al., Nucl. Med. Biol., 28: 409, 2001), imaging techniques and radioimmunoguided surgery or labels that allow for in vivo imaging or detection of specific antibody/antigen complexes. Antibodies may also be conjugated with toxins to provide an immunotoxin (see, Kreitman, R. J Adv. Drug Del. Rev., 31: 53, 1998).

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method first described by Kohler, et al., Nature, 256: 495, 1975, or can be made by recombinant DNA methods (see, e.g., U.S. Pat. No. 4,816,567, Cabilly, et al.). The “monoclonal antibodies” can also be isolated from phage antibody libraries using the techniques described in Clackson, et al., 624-628, 1991; Marks, et al., J. Mol. Biol., 222: 581-597, 1991, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly, et al., supra; Morrison, et al., Proc. Natl. Acad. Sci. U.S.A., 81: 6851-6855, 1984).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones, et al., Nature, 321: 522-525, 1986; Reichmann, et al., Nature, 332: 323-329, 1988; Presta, Curr. Op. Struct. Biol., 2: 593-596, 1992. The humanized antibody includes a Primatized™ antibody wherein the antigen-binding region of the antibody is derived from an antibody produced by immunizing macaque monkeys with the antigen of interest.

“Non-immunogenic in a human” means that upon contacting the polypeptide of interest in a physiologically acceptable carrier and in a therapeutically effective amount with the appropriate tissue of a human, no state of sensitivity or resistance to the polypeptide of interest is demonstrable upon the second administration of the polypeptide of interest after an appropriate latent period (e.g., 8 to 14 days).

A “neutralizing antibody” is meant an antibody which is able to block or significantly reduce an effector function of wild type or recombinant PEDF-R For example, a neutralizing antibody can inhibit or reduce PEDF-R activation by an agonist antibody, as determined, for example, in a neurite survival assays, a PEDF-R binding assay, or other assays taught herein or known in the art.

Methods of producing polyclonal and monoclonal antibodies that react specifically with the PEDF-R proteins are known to those of skill in the art (see, e.g., Coligan, Current Protocols in Immunology, 1991; Harlow, et al., supra; Goding, Monoclonal Antibodies: Principles and Practice (2d ed.), 1986; Kohler, et al., Nature, 256: 495-497, 1975. Such techniques include antibody preparation by selection of antibodies from libraries of recombinant antibodies in phage or similar vectors, as well as preparation of polyclonal and monoclonal antibodies by immunizing rabbits or mice (see, e.g., Huse, et al., Science, 246: 1275-1281, 1989; Ward, et al., Nature, 341: 544-546, 1989).

A number of immunogens comprising portions of PEDF-R protein can be used to produce antibodies specifically reactive with PEDF-R protein. For example, recombinant PEDF-R protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein can also be used either in pure or impure form. The product is then injected into an animal capable of producing antibodies. Either monoclonal or polyclonal antibodies can be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies can be obtained by various techniques familiar to those skilled in the art. Briefly, spleen cells from an animal immunized with a desired antigen are immortalized, commonly by fusion with a myeloma cell (see, Kohler, et al., Eur. J. Immunol., 6: 511-519, 1976). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one can isolate DNA sequences which encode a monoclonal antibody or a binding fragment thereof by screening a DNA library from human B cells according to the general protocol outlined by Huse, et al., Science, 246: 1275-1281, 1989.

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-PEDF-R proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular PEDF-R ortholog, such as human PEDF-R, can also be made, by subtracting out other cross-reacting orthologs from a species such as a non-human mammal.

Once the specific antibodies against PEDF-R protein are available, the protein can be detected by a variety of immunoassay methods. In addition, the antibody can be used therapeutically as PEDF-R modulators. For a review of immunological and immunoassay procedures, see Basic and Clinical Immunology (Stites & Terr eds., 7th ed., 1991). Moreover, the immunoassays of the present invention can be performed in any of several configurations, which are reviewed extensively in Enzyme Immunoassay (Maggio, ed., 1980); and Harlow & Lane, supra.

In a further embodiment, antibodies or antibody fragments can be isolated from antibody phage libraries generated using the techniques described in McCafferty, et al., Nature, 348: 552-554, 1990; Clackson, et al., Nature, 352: 624-628, 1991; Marks, et al., J. Mol. Biol., 222: 581-597, 1991, describe the isolation of murine and human antibodies, respectively, using phage libraries. Subsequent publications describe the production of high affinity (nM range) human antibodies by chain shuffling (Mark, et al., Bio/Technology, 10: 779-783, 1992), as well as combinatorial infection and in vivo recombination as a strategy for constructing very large phage libraries (Waterhouse, et al., Nuc. Acids. Res., 21: 2265-2266, 1993). Thus, these techniques are viable alternatives to traditional monoclonal antibody hybridoma techniques for isolation of monoclonal antibodies.

The DNA also can be modified, for example, by substituting the coding sequence for human heavy- and light-chain constant domains in place of the homologous murine sequences (Cabilly, et al., supra; Morrison, et al., Proc. Nat. Acad. Sci. U.S.A., 81: 6851, 1984), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Chimeric or hybrid antibodies also can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate.

Methods for humanizing non-human antibodies are well known in the art. Generally, a humanized antibody has one or more amino acid residues introduced into it from a source which is nonhuman. These non-human amino acid residues are often referred to as “import” residues, which are typically taken from an “import” variable domain. Humanization can be essentially performed following the method of Winter and co-workers (Jones, et al., Nature, 321: 522-525, 1986; Riechmann, et al., Nature, 332: 323-327, 1988; Verhoeyen, et al., Science, 239: 1534-1536, 1988), by substituting rodent CDRs or CDR sequences for the corresponding sequences of a human antibody. Accordingly, such “humanized” antibodies are chimeric antibodies (Cabilly, et al., supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims, et al., J Immunol., 151: 2296, 1993; Chothia, et al, J. Mol. Biol., 196: 901, 1987). Another method uses a particular framework derived from the consensus sequence of all human antibodies of a particular subgroup of light or heavy chains. The same framework can be used for several different humanized antibodies (Carter, et al., Proc. Natl. Acad. Sci. U.S.A., 89: 4285, 1992; Presta, et al., J. Immunol., 151: 2623, 1993).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits, et al., Proc. Natl. Acad. Sci. U.S.A., 90: 2551, 1993; Jakobovits, et al., Nature, 362: 255-258, 1993; Bruggermann, et al., Year in Immuno., 7: 33, 1993. Human antibodies can also be produced in phage—display libraries (Hoogenboom, et al., J. Mol. Biol., 227: 381, 1991; Marks, et al., J. Mol. Biol., 222: 581, 1991).

Bispecific antibodies (BsAbs) are antibodies that have binding specificities for at least two different antigens. BsAbs can be used as tumor targeting or imaging agents and can be used to target enzymes or toxins to a cell possessing the PEDF-R. Such antibodies can be derived from full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein, et al., Nature, 305: 537-539, 1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker, et al., EMBO J., 10: 3655-3659,1991.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details of generating bispecific antibodies see, for example, Suresh, et al., Methods in Enzymology, 121: 210, 1986.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies can be made using any convenient cross-linking methods. Suitable crosslinking agents are well known in the art, and are disclosed in U.S. Pat. No. 4,676,980, along with a number of cross-linking techniques.

Techniques for generating bispecific antibodies from antibody fragments have also been described in the literature. The following techniques can also be used for the production of bivalent antibody fragments which are not necessarily bispecific. According to these techniques, Fab′-SH fragments can be recovered from E. coli, which can be chemically coupled to form bivalent antibodies. Shalaby, et al., J. Exp. Med., 175: 217-225, 1992, describe the production of a fully humanized BsAb F(ab′)₂ molecule. Each Fab′ fragment was separately secreted from E. coli and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also Rodriguez, et al., Int. J. Cancers, (Suppl.) 7: 45-50, 1992.

Various techniques for making and isolating bivalent antibody fragments directly from recombinant cell culture have also been described. For example, bivalent heterodimers have been produced using leucine zippers. Kostelny, et al., J Immunol., 148: 1547-1553, 1992. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger, et al., Proc. Natl. Acad. Sci. U.S.A., 90: 6444-6448, 1993, has provided an alternative mechanism for making BsAb fragments. The fragments comprise a heavy-chain variable domain (VH) connected to a light-chain variable domain (V_{L}) by a linker which is too short to allow pairing between the two domains on the same chain. Accordingly, the V_{H} and V_{L} domains of one fragment are forced to pair with the complementary V_{H} and V_{L} domains of another fragment, thereby forming two antigen-binding sites. Another strategy for making BsAb fragments by the use of single-chain Fv (sFv) dimers has also been reported. See Gruber, et al., J. Immunol., 152: 5368, 1994.

Gene amplification and/or expression can be measured in a sample directly, for example, by conventional Southern blotting, Northern blotting to quantitate the transcription of mRNA (Thomas, Proc. Natl. Acad. Sci. U.S.A., 77: 5201-5205, 1980), dot blotting (DNA analysis), or in situ hybridization, using an appropriately labeled probe, based on the sequences provided herein. Various labels can be employed, most commonly radioisotopes, particularly ³²P. However, other techniques can also be employed, such as using biotin-modified nucleotides for introduction into a polynucleotide. The biotin then serves as the site for binding to avidin or antibodies, which can be labeled with a wide variety of labels, such as radionuclides, fluorescers, enzymes, or, the like. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of tissue sections and assay of cell culture or body fluids, to quantitate directly the expression of gene product. With immunohistochemical, staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu, et al., Am. J. Clin. Path., 75: 734-738, 1980.

Antibodies useful for immunohistochemical staining and/or assay of sample fluids can be either monoclonal or polyclonal, and can be prepared as described herein.

When PEDF-R is produced in a recombinant cell other than one of human origin, the PEDF-R is completely free of proteins or polypeptides of human origin. However, it is necessary to purify PEDF-R from recombinant cell proteins or polypeptides to obtain preparations that are substantially homogeneous as to PEDF-R. As a first step, the culture medium or lysate can be centrifuged to remove particulate cell debris. PEDF-R can then be purified from contaminant soluble proteins and polypeptides with the following procedures, which are exemplary of suitable purification procedures: by fractionation on an ion-exchange column; ethanol precipitation; reverse phase HPLC; chromatography on silica; chromatofocusing; immunoaffinity; epitope-tag binding resin; SDS-PAGE; ammonium sulfate precipitation; gel filtration using, for example, Sephadex G-75; and protein A Sepharose columns to remove contaminants such as IgG.

PEDF-R variants in which residues have been deleted, inserted, or substituted are recovered in the same fashion as wild type PEDF-R, taking account of any substantial changes in properties occasioned by the variation. Immunoaffinity resins, such as a monoclonal anti-PEDF-R resin, can be employed to absorb the PEDF-R variant by binding it to at least one remaining epitope.

A protease inhibitor such as phenyl methyl sulfonyl fluoride (PMSF) also can be useful to inhibit proteolytic degradation during purification, and antibiotics can be included to prevent the growth of adventitious contaminants.

Covalent modifications of PEDF-R polypeptides are included within the scope of this invention. Both wild type PEDF-R and amino acid sequence variants of the PEDF-R can be covalently modified. One type of covalent modification of the PEDF-R is introduced into the molecule by reacting targeted amino acid residues of the PEDF-R with an organic derivatizing agent that is capable of reacting the N-terminal residue, the C-terminal residue, or with selected side chains.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with dimethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing a-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed under alkaline conditions because of the high pKa of the guanidine functional group. Furthermore, these reagents can react with the groups of lysine as well as with the arginine epsilon-amino group.

The specific modification of tyrosyl residues can be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method being suitable.

Carboxyl side groups (aspartyl or glutamyl) are selectively modified by reaction with carbodiimides (RN═C═NR′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking PEDF-R to a water-insoluble support matrix or surface for use in the method for purifying anti-PEDF-R antibodies, and vice versa. Commonly used crosslinking agents include, e.g., 1,1-bis(diazoacetyl)-2-phenyletha-ne, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-((p-azidophenyl)dithio)propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure And Molecular Properties, W.H. Freeman & Co., San Francisco, pp. 79-86, 1983), acetylation of the N-terminal amine, and amidation of any C-terminal carboxyl group.

Another type of covalent modification of the PEDF-R polypeptide included within the scope of this invention comprises altering the native glycosylation pattern of the polypeptide. By altering is meant deleting one or more carbohydrate moieties found in native PEDF-R, and/or adding one or more glycosylation sites that are not present in the native PEDF-R.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars Naceylgalactosamine, galactose, or xylose to a hydroxylamino acid, most commonly serine or threonine, although 5-hydroxyproline or 5-hydroxylysine can also be used.

Addition of glycosylation sites to the PEDF-R polypeptide is conveniently accomplished by altering the amino acid sequence such that it contains one or more of the above-described tripeptide sequences (for N-linked glycosylation sites). The alteration can also be made by the addition of, or substitution by, one or more serine or threonine residues to the native PEDF-R sequence (for O-linked glycosylation sites). For case, the PEDF-R amino acid sequence is preferably altered through changes at the DNA level, particularly by mutating the DNA encoding the PEDF-R polypeptide at preselected bases such that codons are generated that will translate into the desired amino acids. The DNA mutation(s) can be made using methods described above and in U.S. Pat. No. 5,364,934, supra.

Another means of increasing the number of carbohydrate moieties on the PEDF-R polypeptide is by chemical or enzymatic coupling of glycosides to the polypeptide. These procedures are advantageous in that they do not require production of the polypeptide in a host cell that has glycosylation capabilities for N- or O-linked glycosylation. Depending on the coupling mode used, the sugar(s) can be attached to (a) arginine and histidine, (b) free carboxyl groups, (c) free sulthydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin, et al., CRC Crit. Rev. Biochem., 259-306, 1981.

Removal of carbohydrate moieties present on the PEDF-R polypeptide can be accomplished chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin, et al., Arch. Biochem. Biophys., 259: 52, 1987; Edge, et al., Anal. Biochem., 118: 131, 1981. Enzymatic cleavage of carbohydrate moieties on polypeptides can be achieved by the use of a variety of endo- and exo-glycosidases as described by Thotakura, et al., Methyl. Enzymol., 138: 350, 1987.

Glycosylation at potential glycosylation sites can be prevented by the use of the compound tunicamycin as described by Duskin, et al., J. Biol. Chem., 257: 3105, 1982. Tunicamycin blocks the formation of protein-N-glycoside linkages.

Another type of covalent modification of PEDF-R comprises linking the PEDF-R polypeptide to one of a variety of nonproteinaceous polymers, e.g. polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

Variants can be assayed as taught herein. A change in the immunological character of the PEDF-R molecule, such as affinity for a given antibody, can be measured by a competitive-type immunoassay. Other potential modifications of protein or polypeptide properties such as redox or thermal stability, hydrophobicity, susceptibility to proteolytic degradation, or the tendency to aggregate with carriers or into multimers are assayed by methods well known in the art.

This invention encompasses chimeric polypeptides comprising PEDF-R fused to a heterologous polypeptide. A chimeric PEDF-R is one type of PEDF-R variant as defined herein. In one preferred embodiment, the chimeric polypeptide comprises a fusion of the PEDF-R with a tag polypeptide which provides an epitope to which an anti-tag antibody or molecule can selectively bind. The epitope-tag is generally provided at the amino- or carboxyl-terminus of the PEDF-R. Such epitope-tagged forms of the PEDF-R are desirable, as the presence thereof can be detected using a labeled antibody against the tag polypeptide. Also, provision of the epitope tag enables the PEDF-R to be readily purified by affinity purification using the anti-tag antibody. Affinity purification techniques and diagnostic assays involving antibodies are described later herein.

Tag polypeptides and their respective antibodies are well known in the art. Examples include the flu HA tag polypeptide and its antibody 12CA5 (Field, et al., Mol. Cell. Biol., 8: 2159-2165, 1988); the c-myc tag and the 8F9, 3C7, 6E10, G4, B7 and 9E10 antibodies thereto (Evan, et al., Molecular and Cellular Biology, 5: 3610-3616, 1985); and the Herpes Simplex virus glycoprotein D (gD) tag and its antibody. Paborsky, et al., Protein Engineering, 3: 547-553, 1990. Other tag polypeptides have been disclosed. Examples include the Flag-peptide (Hopp, et al., BioTechnology, 6: 1204-1210, 1988); the KT3 epitope peptide (Martin, et al., Science, 255: 192-194, 1992); and α-tubulin epitope peptide (Skinner, et al., J. Biol. Chem., 266: 15163-15166, 1991); and the T7 gene 10 protein peptide tag. Lutz-Freyermuth, et al., Proc. Natl. Acad. Sci. U.S.A., 87: 6393-6397, 1990. Once the tag polypeptide has been selected, an antibody thereto can be generated using the techniques disclosed herein. A C-terminal poly-histidine sequence tag is preferred. Poly-histidine sequences allow isolation of the tagged protein by Ni-NTA chromatography for example as described in Lindsay, et al., Neuron, 17: 571-574, 1996.

The general methods suitable for the construction and production of epitope-tagged PEDF-R are the same as those disclosed herein above. PEDF-R tag polypeptide fusions are most conveniently constructed by fusing the cDNA sequence encoding the PEDF-R portion in-frame to the tag polypeptide DNA sequence and expressing the resultant DNA fusion construct in appropriate host cells. Ordinarily, when preparing the PEDF-R-tag polypeptide chimeras of the present invention, nucleic acid encoding the PEDF-R will be fused at its 3′ end to nucleic acid encoding the N-terminus of the tag polypeptide, however 5′ fusions are also possible.

Epitope-tagged PEDF-R can be conveniently purified by affinity chromatography using the anti-tag antibody. The matrix to which the affinity antibody is attached is most often agarose, but other matrices are available (e.g. controlled pore glass or poly(styrenedivinyl)benzene)-. The epitope-tagged PEDF-R can be eluted from the affinity column by varying the buffer pH or ionic strength or adding chaotropic agents, for example.

Chimeras constructed from a receptor sequence linked to an appropriate immunoglobulin constant domain sequence (immunoadhesins) are known in the art. Immunoadhesins reported in the literature include fusions of the T cell receptor (Gascoigne, et al., Proc. Natl. Acad. Sci. U.S.A., 84: 2936-2940, 1987); CD4 (Capon, et al., Nature, 337: 525-531, 1989; Traunecker, et al., Nature, 339: 68-70, 1989; Zettmeissl, et al., DNA Cell Biol. U.S.A, 9: 347-353, 1990; Byrn, et al., Nature, 344: 667-670, 1990); L-selectin (homing receptor) ((Watson, et al., J. Cell. Biol., 110: 2221-2229, 1990; Watson, et al., Nature, 349: 164-167, 1991); CD44 (Aruffo, et al., Cell, 61: 1303-1313, 1990); CD28 and B7 (Linsley, et al., J. Exp. Med., 173: 721-730, 1991); CTLA-4 (Lisley, et al., J. Exp. Med., 174: 561-569, 1991); CD22 (Stamenkovic, et al., Cell, 66:1133-1144, 1991); TNF receptor (Ashkenazi, et al., Proc. Natl. Acad. Sci. U.S.A., 88: 10535-10539, 1991; Lesslauer, et al., Eur. J. Immunol., 27: 2883-2886, 1991; Peppel, et al., J. Exp. Med., 174:1483-1489, 1991); NP receptors (Bennett, et al., J. Biol. Chem., 266: 23060-23067, 1991); and IgE receptor a (Ridgway, et al., J. Cell. Biol., 115: abstr. 1448,1991). See also U.S. Pat. Nos. 6,406,697, 6,403,769, 5,998,598, and 5,116,964. These references are hereby incorporated by reference in their entirety for all purposes.

The simplest and most straightforward immunoadhesin design combines the binding region(s) of the “adhesion” protein with the hinge and Fc regions of an immunoglobulin heavy chain. Ordinarily, when preparing the PEDF-Rimmunoglobulin chimeras of the present invention, nucleic acid encoding the extracellular domain of the PEDF-R will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge and CH2 and CH3 domains of the constant region of an immunoglobulin heavy chain. Fusions are also made to the C-terminus of the Fc portion of a constant domain, or immediately Nterminal to the CH1 of the heavy chain or the corresponding region of the light chain.

The precise site at which the fusion is made is not critical; particular sites are well known and can be selected in order to optimize the biological activity, secretion or binding characteristics of the PEDF-R immunoglobulin chimeras.

In some embodiments, the PEDF-R immunoglobulin chimeras are assembled as monomers, or hetero- or homo-multimer, and particularly as dimers or tetramers, essentially as illustrated in WO 91/08298.

In a preferred embodiment, the PEDF-R extracellular domain sequence is fused to the N-terminus of the C-terminal portion of an antibody (in particular the Fc domain), containing the effector functions of an immunoglobulin. It is possible to fuse the entire heavy chain constant region to the PEDF-R extracellular domain sequence. However, more preferably, a sequence beginning in the hinge region just upstream of the papain cleavage site (which defines IgG Fc chemically; residue 216, taking the first residue of heavy chain constant region to be 114, or analogous sites of other immunoglobulins) is used in the fusion. In a particularly preferred embodiment, the PEDF-R amino acid sequence is fused to the hinge region and CH2 and CH3, or to the CH1, hinge, CH2 and CH3 domains of an IgG₁, IgG₂, or IgG₃ heavy chain. The precise site at which the fusion is made is not critical, and the optimal site can be determined by routine experimentation.

In some embodiments, the PEDF-R immunoglobulin chimeras are assembled as multimer, and particularly as homo-dimers or -tetramers. Generally, these assembled immunoglobulins will have known unit structures. A basic four chain structural unit is the form in which IgG, IgD, and IgE exist. A four unit is repeated in the higher molecular weight immunoglobulins; IgM generally exists as a pentamer of basic four units held together by disulfide bonds. IgA globulin, and occasionally IgG globulin, can also exist in multimeric form in serum. In the case of multimer, each four unit can be the same or different.

Alternatively, the PEDF-R extracellular domain sequence can be inserted between immunoglobulin heavy chain and light chain sequences such that an immunoglobulin comprising a chimeric heavy chain is obtained. In this embodiment, the PEDF-R sequence is fused to the 3′ end of an immunoglobulin heavy chain in each arm of an immunoglobulin, either between the hinge and the CH2 domain, or between the CH2 and CH3 domains. Similar constructs have been reported by Hoogenboom, et al., Mol. Immunol., 28: 1027-1037, 1991.

Although the presence of an immunoglobulin light chain is not required in the immunoadhesins of the present invention, an immunoglobulin light chain might be present either covalently associated to a PEDF-R-immunoglobulin heavy chain fusion polypeptide, or directly fused to the PEDF-R extracellular domain. In the former case, DNA encoding an immunoglobulin light chain is typically coexpressed with the DNA encoding the PEDF-R immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567.

In a preferred embodiment, the immunoglobulin sequences used in the construction of the immunoadhesins of the present invention are from an IgG immunoglobulin heavy chain constant domain. For human immunoadhesins, the use of human IgG₁ and IgG₃ immunoglobulin sequences is preferred. A major advantage of using IgG₁ is that IgG₃, immunoadhesins can be purified efficiently on immobilized protein A. In contrast, purification of IgG₃ requires protein G, a significantly less versatile medium. However, other structural and functional properties of immunoglobulins should be considered when choosing the Ig fusion partner for a particular immunoadhesin construction. For example, the IgG₃ hinge is longer and more flexible, so it can accommodate larger adhesion domains that cannot fold or function properly when fused to IgG₁. Another consideration can be valency; IgG immunoadhesins are bivalent homodimers, whereas Ig subtypes like IgA and IgM can give rise to dimeric or pentameric structures, respectively, of the basic Ig homodimer unit. For PEDF-R immunoadhesins designed for in vivo application, the pharmacokinetic properties and the effector functions specified by the Fc region are important as well. Although IgG₁, IgG₂ and IgG₄ all have in vivo half-lives of 21 days, their relative potencies at activating the complement system are different. IgG₄ does not activate complement, and IgG2 is significantly weaker at complement activation than IgG1. Moreover, unlike IgG₁, IgG₂ does not bind to Fe receptors on mononuclear cells or neutrophils. While IgG₃ is optimal for complement activation, its in vivo half-life is approximately one third of the other IgG isotypes. Another important consideration for immunoadhesins designed to be used as human therapeutics is the number of allotypic variants of the particular isotype. In general, IgG isotypes with fewer serologically-defined allotypes are preferred. For example, IgG₁ has only four serologically-defined allotypic sites, two of which (Gim and 2) are located in the Fe region; and one of these sites G1m1, is non-immunogenic. In contrast, there are 12 serologically defined allotypes in IgG₃, all of which are in the Fc region; only three of these sites (G3 m5, 11 and 21) have one allotype which is nonimmunogenic. Thus, the potential immunogenicity of a γ3 immunoadhesin is greater than that of a γ1 immunoadhesin.

With respect to the parental immunoglobulin, a useful joining point is just upstream of the cysteines of the hinge that form the disulfide bonds between the two heavy chains. In a frequently used design, the codon for the C-terminal residue of the PEDF-R part of the molecule is placed directly upstream of the codons for the sequence DKTHTCPPCP of the IgG1 hinge region.

The general methods suitable for the construction and expression of immunoadhesins are the same as those disclosed herein above with regard to PEDF-R immunoadhesins are most conveniently constructed by fusing the cDNA sequence encoding the PEDF-R portion in-frame to an Ig cDNA sequence. However, fusion to genomic Ig fragments can also be used (see, e.g., Gascoigne, et al., Proc. Natl. Acad. Sci. U.S.A., 84: 2936-2940, 1987; Aruffo, et al., Cell, 61: 1303-1313, 1990; Stamenkovic, et al., Cell, 66: 1133-1144, 1991). The latter type of fusion requires the presence of Ig regulatory sequences for expression. cDNAs encoding IgG heavy-chain constant regions can be isolated based on published sequence from cDNA libraries derived from spleen or peripheral blood lymphocytes, by hybridization or by polymerase chain reaction (PCR) techniques. The cDNAs encoding the PEDF-R and Ig parts of the immunoadhesin are inserted in tandem into a plasmid vector that directs efficient expression in the chosen host cells. For expression in mammalian cells, pRK5-based vectors (Schall, et al., Cell, 61: 361-370, 1990) and CDM8-based vectors (Seed, Nature, 329: 840, 1989) can be used. The exact junction can be created by removing the extra sequences between the designed junction codons using oligonucleotide-directed deletional mutagenesis (Zoller, et al., Nucleic Acids Res., 10: 6487, 1982; Capon, et al., Nature, 337: 525-531, 1989). Synthetic oligonucleotides can be used, in which each half is complementary to the sequence on either side of the desired junction; ideally, these are 36 to 48-mers. Alternatively, PCR techniques can be used to join the two parts of the molecule in-frame with an appropriate vector.

The choice of host cell line for the expression of PEDF-R immunoadhesins depends mainly on the expression vector. Another consideration is the amount of protein that is required. Milligram quantities often can be produced by transient transfections. For example, the adenovirus EIA transformed 293 human embryonic kidney cell line can be transfected transiently with pRK5-based vectors by a modification of the calcium phosphate method to allow efficient immunoadhesin expression. CDM8 based vectors can be used to transfect COS cells by the DEAE-dextran method (Aruffo, et al., Cell, 61: 1303-1313, 1990; Zettmeissl, et al., DNA Cell Biol. US., 9: 347-353, 1990). If larger amounts of protein are desired, the immunoadhesin can be expressed after stable transfection of a host cell line. For example, a pRK5-based vector can be introduced into Chinese hamster ovary (CHO) cells in the presence of an additional plasmid encoding dihydrofolate reductase (DHFR) and conferring resistance to G418. Clones resistant to G418 can be selected in culture; these clones are grown in the presence of increasing levels of DHFR inhibitor methotrexate; clones are selected, in which the number of gene copies encoding the DHFRand immunoadhesin sequences is co-amplified. If the immunoadhesin contains a hydrophobic leader sequence at its N-terminus, it is likely to be processed and secreted by the transfected cells. The expression of immunoadhesins with more complex structures can require uniquely suited host cells; for example, components such as light chain or J chain can be provided by certain myeloma or hybridoma cell hosts (Gascoigne, et al., supra, 1987; Martin, et al., J. Virol., 67: 3561-3568, 1993).

Immunoadhesins can be conveniently purified by affinity chromatography. The suitability of protein A as an affinity ligand depends on the species and isotype of the immunoglobulin Fe domain that is used in the chimera. Protein A can be used to purify immunoadhesins that are based on human γ1, γ2, or γ4 heavy chains (Lindmark, et al., J Immunol Meth., 62: 1-13, 1983). Protein G is recommended for all mouse isotypes and for human γ3 (Guss, et al., EMBO J, 5: 1567-1575, 1986). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. The conditions for binding an immunoadhesin to the protein A or G affinity column are dictated entirely by the characteristics of the Fe domain; that is, its species and isotype. Generally, when the proper ligand is chosen, efficient binding occurs directly from unconditioned culture fluid. One distinguishing feature of immunoadhesins is that, for human γ1 molecules, the binding capacity for protein A is somewhat diminished relative to an antibody of the same Fe type. Bound immunoadhesin can be efficiently eluted either at acidic pH (at or above 3.0), or in a neutral pH buffer containing a mildly chaotropic salt. This affinity chromatography step can result in an immunoadhesin preparation that is >95% pure.

Other methods known in the art can be used in place of, or in addition to, affinity chromatography on protein A or G to purify immunoadhesins. Immunoadhesins behave similarly to antibodies in thiophilic gel chromatography (Hutchens, et al., Anal. Biochem., 159: 217-226, 1986) and immobilized metal chelate chromatography (Al-Mashikhi, et al., J Dairy Sci., 71: 1756-1763, 1988). In contrast to antibodies, however, their behavior on ion exchange columns is dictated not only by their isoelectric points, but also by a charge dipole that can exist in the molecules due to their chimeric nature.

If desired, the immunoadhesins can be made bispecific. Thus, the immunoadhesins of the present invention can combine a PEDF-R extracellular domain and a domain, such as the extracellular domain, of another cytokine or neurotrophic factor receptor subunit. Exemplary cytokine receptors from which such bispecific immunoadhesin molecules can be made include TPO (or mpl ligand), EPO, G-CSF, IL-4, IL-7, GH, PRL, IL-3, GM-CSF, IL-5, IL-6, LIF, OSM, CNTF, GDNF and IL-2 receptors. For bispecific molecules, trimeric molecules, composed of a chimeric antibody heavy chain in one arm and a chimeric antibody heavy chain-light chain pair in the other amine of their antibody-like structure are advantageous, due to ease of purification. In contrast to antibody-producing quadromas traditionally used for the production of bispecific immunoadhesins, which produce a mixture of ten tetramers, cells transfected with nucleic acid encoding the three chains of a trimeric immunoadhesin structure produce a mixture of only three molecules, and purification of the desired product from this mixture is correspondingly easier.

The PEDF-R protein and PEDF-R gene are believed to find ex vivo or in vivo therapeutic use for administration to a mammal, particularly humans, in the treatment of diseases or disorders, related to PEDF activity or benefited by PEDF-R responsiveness. Conditions particularly amenable to treatment with the embodiments of the invention are those related to, for example, neuronal survival in the CNS and retina, differentiation in the CNS and retina, and proliferative disorders. The patient is administered an effective amount of PEDF-R, PEDF-R agonists (e.g. PEDF), PEDF-R antagonists (which compete with and bind endogenous PEDF), or anti-PEDF-R antibodies. The present invention also provides for pharmaceutical compositions comprising PEDF-R, PEDF-R agonists (e.g. PEDF), PEDF-R antagonists (which compete with and bind endogenous PEDF), or anti-PEDF-R antibodies in a suitable pharmacologic carrier. The PEDF-R, PEDF-R agonists (e.g. PEDF), PEDF-R antagonists (which compete with and bind endogenous PEDF), or anti-PEDF-R antibodies can be administered systemically or locally. Applicable to the methods taught herein, the receptor protein can be optionally administered concomitantly with (or in complex with) PEDF or other PEDF-R ligands. As taught herein, PEDF-R can be provided to target cells in the absence of PEDF to increase the responsiveness of those cells to subsequently administered PEDF ligands.

Certain conditions can benefit from an increase in PEDF (or other PEDF-R ligand) responsiveness. It can therefore be beneficial to increase the number of or binding affinity of PEDF-R in cells of patients suffering from such conditions. This can be achieved through, for example, gene therapy using PEDF-R encoding nucleic acid. Selective expression of recombinant PEDF-R in appropriate cells could be achieved using PEDF-R genes controlled by tissue specific or inducible promoters or by producing localized infection with replication defective viruses carrying a recombinant PEDF-R gene. Conditions which can benefit from increased sensitivity to PEDF include, but are not limited to, PEDF-related disorders such as neural disorders.

A disease or medical disorder is considered to be nerve or neural damage if the survival or function of nerve cells and/or their axonal processes is compromised. Such nerve damage occurs as the result conditions including (a) Physical injury, which causes the degeneration of the axonal processes and/or nerve cell bodies near the site of the injury; (b) Ischemia, as a stroke; (c) Exposure to neurotoxins, such as the cancer and AIDS chemotherapeutic agents such as cisplatin and dideoxycytidine (ddC), respectively; (d) Chronic metabolic diseases, such as diabetes or renal dysfunction; and (e) Neurodegenerative diseases, for examples diseases triggered by the death of cerebellar neurons. Neurodegenerative diseases treatable by the methods of the present invention include, for example, Parkinson's disease, Alzheimer's disease, and Amyotrophic Lateral Sclerosis (ALS, also known as Lou Gherig's disease), which cause the degeneration of specific neuronal populations. Conditions involving nerve damage include Parkinson's disease, Alzheimer's disease, Amyotrophic Lateral Sclerosis, stroke, diabetic polyneuropathy, toxic neuropathy, and physical damage to the nervous system such as that caused by physical injury of the brain and spinal cord or crush or cut injuries to the arm and hand or other parts of the body, including temporary or permanent cessation of blood flow to parts of the nervous system, as in stroke. The present invention provides methods for treating such diseases by administering therapeutic compounds, e.g., pharmaceutical compositions comprising one or more selected compounds of the present invention, to a subject.

The present invention also provides methods for treating ocular disease. Ocular-related disorders appropriate for treatment using the present inventive materials and methods include, but are not limited to, diabetic retinopathies, proliferative retinopathies, retinopathy of prematurity, retinal vascular diseases, vascular anomalies, age-related macular degeneration and other acquired disorders, endophthalmitis, infectious diseases, inflammatory diseases, AIDS-related disorders, ocular ischemia syndrome, pregnancy-related disorders, peripheral retinal degenerations, retinal degenerations, toxic retinopathies, cataracts, retinal tumors, corneal neovascularization, choroidal tumors, choroidal disorders, choroidal neovascularization, neovascular glaucoma, vitreous disorders, retinal detachment and proliferative vitreoretinopathy, cyclitis, non-penetrating trauma, penetrating trauma, post-cataract complications, Hippel-Lindau Disease, dry eye, inflammatory optic neuropathies, macular edema, pterygium, iris neovascularization, and surgical-induced disorders.

Selected compounds and compositions of the present invention are particularly useful for ocular disease caused by ocular neovascularization, i.e., the abnormal proliferation of new blood vessels within the eye. Accordingly, in one aspect of the present invention, neovascularization of the choroids is treated using the compounds of the present invention. The choroid is a thin, vascular membrane located under the retina. Age-related macular degeneration, one of the leading causes of blindness, is characterized by the sprouting of choroids vessels into the subretinal space of the macula and is thus treatable by the methods and compositions of the present invention. Abnormal neovascularization from, for example, photocoagulation, anterior ischemic optic neuropathy, Best's disease, choroidal hemangioma, metallic intraocular foreign body, choroidal nonperfusion, choroidal osteomas, choroidal rupture, bacterial endocarditis, choroideremia, chronic retinal detachment, drusen, deposit of metabolic waste material, endogenous Candida endophthalmitis, neovascularization at ora serrata, operating microscope burn, punctate inner choroidopathy, radiation retinopathy, retinal cryoinjury, retinitis pigmentosa, retinochoroidal coloboma, rubella, subretinal fluid drainage, tilted disc syndrome, Taxoplasma Tetinochoroiditis, or tuberculosis can treated with the methods and compounds of the present invention.

Neovascularization of the cornea is also appropriate for treatment by the method of the present invention. The cornea is a projecting, transparent section of the fibrous tunic, the outer most layer of the eye. The outermost layer of the cornea contacts the conjunctiva, while the innermost layer comprises the endothelium of the anterior chamber. Corneal neovascularization stems from, for example, ocular injury, surgery, infection, improper wearing of contact lenses, and diseases such as, for example, corneal dystrophies.

Alternatively, the compositions and methods of the present invention are used to treat ocular neovascularization of the retina. Retinal neovascularization is an indication associated with numerous ocular diseases and disorders, many of which are named above. Preferably, the neovascularization of the retina treated in accordance with the present inventive method is associated with diabetic retinopathy. Common causes of retinal neovascularization include ischemia, viral infection, and retinal damage. Neovascularization of the retina can lead to macular edema, subretinal discoloration, and/or scarring.

The compositions and methods of the present invention can be used to promote the development, maintenance, regeneration, migration, or process-outgrowth of neurons in vivo, including central (brain and spinal chord), peripheral (sympathetic, parasympathetic, sensory, and enteric neurons), and motoneurons. The ligands, agonists and antagonists can accordingly be used to stimulate or inhibit these activities associated with neurodegenerative conditions and conditions involving trauma and injury to the nervous system. Consequently, selected compositions of the present invention can be utilized in methods for the diagnosis and/or treatment of a variety of neurologic diseases and disorders.

In some embodiments of the invention, selected compounds can be administered to patients in whom the nervous system has been damaged by trauma, surgery, stroke, ischemia, infection, metabolic disease, nutritional deficiency, malignancy, or toxic agents, to promote the survival or growth of neurons. Selected compounds can be used to treat human neurodegenerative disorders, such as Alzheimer's disease, Parkinson's disease, epilepsy, demyelinating diseases, such as multiple sclerosis, Huntington's chorea, Down's Syndrome, nerve deafness, Meniere's disease, and other disorders of the cerebellum (Hefti, Neurobiol., 25: 1418-35, 1994; Marsden, Lancet, 335: 948-952, 1990; Agid, Lancet, 337: 1321-1327, 1991; Wexler, et al., Ann. Rev. Neurosci., 14: 503-529, 1991). Selected compounds can be used as cognitive enhancer; to enhance learning particularly in dementias or trauma, since they can promote axonal outgrowth and synaptic plasticity. Selected compounds can be used in bacterial and viral infections of the nervous system, deficiency diseases, such as Wernicke's disease and nutritional polyneuropathy, progressive supranuclear palsy, Shy Drager's syndrome, multistem degeneration and olivo ponto cerebellar atrophy, and peripheral nerve damage.

Selected compounds of the invention can also be used to treat neuropathy, and especially peripheral neuropathy. “Peripheral neuropathy” refers to a disorder affecting the peripheral nervous system, most often manifested as one or a combination of motor, sensory, sensorimotor, or autonomic neural dysfunction. The wide variety of morphologies exhibited by peripheral neuropathies can each be attributed uniquely to an equally wide number of causes. For example, peripheral neuropathies can be genetically acquired, can result from a systemic disease, or can be induced by a toxic agent. Examples include but are not limited to distal sensorimotor neuropathy, or autonomic neuropathies such as reduced motility of the gastrointestinal tract or atony of the urinary bladder. Examples of neuropathies associated with systemic disease include post-polio syndrome; examples of hereditary neuropathies include Charcot-Marie-Tooth disease, Refsum's disease, Abetalipoproteinemia, Tangier disease, Krabbe's disease, Metachromatic leukodystrophy, Fabry's disease, and Dejerine-Sottas syndrome; and examples of neuropathies caused by a toxic agent include those caused by treatment with a chemotherapeutic agent such as vincristine, cisplatin, methotrexate, or 3′-azido-3′-deoxythymidi-ne.

The development of a vascular supply, commonly referred to as angiogenesis or neovascularization, is essential for the growth, maturation, and maintenance of normal tissues, including neuronal tissues. It is also required for wound healing and the rapid growth of solid tumors and is involved in a variety of other pathological conditions. Current concepts of angiogenesis, based in large part on studies on the vascularization of tumors, suggest that cells secrete angiogenic factors which induce endothelial cell migration, proliferation, and capillary formation. Numerous factors have been identified which induce vessel formation in vitro or in vivo in animal models. These include FGF-α, FGF-β, TGF-α, TNF-α, VPF or VEGF, monobutyrin, angiotropin, angiogenin, hyaluronic acid degradation products, and more recently, B61 for TNF-α induced angiogenesis (Pandey, et al., Science, 268: 567-569, 1995). The major development of the vascular supply occurs during embryonic development, at ovulation during formation of the corpus luteum, and during wound and fracture healing. Many pathological disease states are characterized by augmented angiogenesis including tumor growth, diabetic retinopathy, neovascular glaucoma, psoriasis, and rheumatoid arthritis. During these processes normally quiescent endothelial cells which line the blood vessels sprout from sites along the vessel, degrade extracellular matrix barriers, proliferate, and migrate to form new vessels. Angiogenic factors, secreted from surrounding tissue, direct the endothelial cells to degrade stromal collagens, undergo directed migration (chemotaxis), proliferate, and reorganize into capillaries.

The present invention includes methods of treating an angiogenesis-mediated disease with an effective amount of one or more of the compositions of the present invention. An effective amount of anti-angiogenic protein is an amount sufficient to inhibit the angiogenesis which results in the disease or condition, thus completely, or partially, alleviating the disease or condition. Alleviation of the angiogenesis-mediated disease can be determined by observing an alleviation of symptoms of the disease, e.g., a reduction in the size of a tumor, or arrested tumor growth. As used herein, the term “effective amount” also means the total amount of each active component of the composition or method that is sufficient to show a meaningful patient benefit, i.e., treatment, healing, prevention or amelioration of the relevant medical condition, or an increase in rate of treatment, healing, prevention or amelioration of such conditions. When applied to a combination, the term refers to combined amounts of the active ingredients that result in the therapeutic effect, whether administered in combination, serially or simultaneously. Angiogenesis-mediated diseases include; but are not limited to, cancers, solid tumors, blood-born tumors (e.g., leukemias), tumor metastasis, benign tumors (e.g., hemangiomas, acoustic neuromas, neurofibromas, organ fibrosis, trachomas, and pyogenic granulomas), rheumatoid arthritis, psoriasis, ocular angiogenic diseases (e.g., diabetic retinopathy, retinopathy of prematurity, macular degeneration, corneal graft rejection, neovascular glaucoma, retrolental fibroplasia, rubeosis), Osler-Webber Syndrome, myocardial angiogenesis, plaque neovascularization, telangiectasia, hemophiliac joints, angiofibroma, and wound granulation. The anti-angiogenic proteins are useful in the treatment of diseases of excessive or abnormal stimulation of endothelial cells. These diseases include, but are not limited to, intestinal adhesions, Crohn's disease, atherosclerosis, scleroderma, fibrosis and hypertrophic scars (i.e., keloids). The anti-angiogenic proteins can be used as a birth control agent by preventing vascularization required for embryo implantation. Anti-angiogenic proteins, such as PEDF or PEDF-like proteins are useful in the treatment of diseases that have angiogenesis as a pathologic consequence such as cat scratch disease (Rochele minalia quintosa) and ulcers (Heliobacterpylori). The anti-angiogenic proteins can also be used to prevent dialysis graft vascular access stenosis, and obesity, e.g., by inhibiting capillary formation in adipose tissue, thereby preventing its expansion. The anti-angiogenic proteins can also be used to treat localized (e.g., nonmetastisized) diseases.

Alternatively, where an increase in angiogenesis is desired, e.g., in wound healing, or in post-infarct heart tissue, antibodies or antisera to the anti-angiogenic proteins can be used to block localized, native anti-angiogenic proteins and processes, and thereby increase formation of new blood vessels so as to inhibit atrophy of tissue. Accordingly, selected compounds of the invention, e.g., neutralizing antibodies, can find further use in promoting or enhancing angiogenesis by receptor inactivation on endothelial or stromal cells. The induction of vascularization is a critical component of the wound healing process. It is desirable to induce neovascularization as early as possible in the course of wound healing, particularly in the case of patients having conditions that tend to retard wound healing, e.g., burns, decubitus ulcers, diabetes, obesity and malignancies. Even normal post-surgical patients will be benefited if they can be released from hospital care at any earlier date because of accelerated wound healing. This invention provides novel compositions and methods for modulating angiogenesis. A patient bearing a wound can be treated by applying an angiogenically active dose of selected compounds of the present invention to the wound. This facilitates the neovascularization of surgical incisions, burns, traumatized tissue, skin grafts, ulcers and other wounds or injuries where accelerated healing is desired. In individuals who have substantially impaired wound healing capacity, thereby lack the ability to provide to the wound site endogenous factors necessary for the process of wound healing, the addition of exogenous compositions of the invention enable-wound healing to proceed in a normal manner. Novel topical compositions containing selected angiogenic compounds of the present invention are provided for use in the inventive method, as are novel articles such as sutures, grafts and dressings containing these selected compounds.

The term “wound” is defined herein as any opening in the skin, mucosa or epithelial linings, most such openings generally being associated with exposed, raw or abraded tissue. There are no limitations as to the type of wound or other traumata that can be treated in accordance with this invention, such wounds including (but are not limited to): first, second and third degree burns (especially second and third degree); surgical incisions, including those of cosmetic surgery; wounds, including lacerations, incisions, and penetrations; and ulcers, e.g., chronic non-healing dermal ulcers, including decubital ulcers (bed-sores) and ulcers or wounds associated with diabetic, dental, hemophilic, malignant and obese patients. Furthermore, normal wound-healing can be retarded by a number of factors, including advanced age, diabetes, cancer, and treatment with anti-inflammatory drugs or anticoagulants, and the proteins described herein can be used to offset the delayed wound-healing effects of such treatments.

In a further embodiment of the invention, patients that suffer from an excess of PEDF-R, hypersensitivity to PEDF, excess PEDF, and the like can be treated by administering an effective amount of anti-sense RNA or anti-sense oligodeoxyribonucleotides corresponding to the PEDF-R gene coding region thereby decreasing expression of PEDF-R.

Transposon insertions or tDNA insertions can be used to inhibit expression of a gene of the present invention. Standard methods are known in the art. Catalytic RNA molecules or ribozymes can also be used to inhibit expression of the genes of the present invention.

Oligonucleotide sequences that include sense RNA and DNA molecules, anti-sense RNA and DNA molecules and ribozymes that function to inhibit the translation of a PEDF-R mRNA are within the scope of the invention. Such molecules are useful in cases where downregulation of PEDF-R expression is desired. Anti-sense RNA and DNA molecules act to directly block the translation of mRNA by binding to targeted mRNA and preventing protein translation. The invention provides methods and antisense oligonucleotide or polynucleotide reagents which can be used to reduce expression of PEDF-R gene products in vitro or in vivo. Administration of the antisense reagents of the invention to a target cell results in reduced PEDF-R activity. As will be apparent to one of skill specific PEDF-R domains can be specifically targeted for inhibition.

Without intending to be limited to any particular mechanism, it is believed that antisense oligonucleotides bind to, and interfere with the translation of, the sense PEDF-R mRNA. Alternatively, the antisense molecule can render the PEDF-R mRNA susceptible to nuclease digestion, interfere with transcription, interfere with processing, localization or otherwise with RNA precursors (“pre-mRNA”), repress transcription of PEDF-R from the PEDF-R gene, or act through some other mechanism. However, the particular mechanism by which the antisense molecule reduces PEDF-R expression is not critical.

The antisense polynucleotides of the invention can comprise an antisense sequence of at least 7 to 10 to typically 20 or more nucleotides that specifically hybridize to a sequence from mRNA encoding PEDF-R or mRNA transcribed from the PEDF-R gene. More often, the antisense polynucleotide of the invention is from about 10 to about 50 nucleotides in length or from about 14 to about 35 nucleotides in length. In other embodiments, antisense polynucleotides are polynucleotides of less than about 100 nucleotides or less than about 200 nucleotides. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate), among other factors. Generally, to assure specific hybridization, the antisense sequence is substantially complementary to the target PEDF-R mRNA sequence. In certain embodiments, the antisense sequence is exactly complementary to the target sequence. The antisense polynucleotides can also include, however, nucleotide substitutions, additions, deletions, transitions, transpositions, or modifications, or other nucleic acid sequences or non-nucleic acid moieties so long as specific binding to the relevant target sequence corresponding to PEDF-R RNA or its gene is retained as a functional property of the polynucleotide.

It will be appreciated that the PEDF-R polynucleotides and oligonucleotides of the invention can be made using nonstandard bases (e.g., other than adenine, cytidine, guanine, thymine, and uridine) or nonstandard backbone structures to provides desirable properties (e.g., increased nuclease-resistance, tighter-binding, stability or a desired TM). Techniques for rendering oligonucleotides nuclease-resistant include those described in PCT publication WO 94/12633. A wide variety of useful modified oligonucleotides may be produced, including oligonucleotides having a peptide-nucleic acid (PNA) backbone (Nielsen et al., 1991, Science 254: 1497) or incorporating 2′-O-methyl ribonucleotides, phosphorothioate nucleotides, methyl phosphonate nucleotides, phosphotriester nucleotides, phosphorothioate nucleotides, phosphoramidates. Still other useful oligonucleotides may contain alkyl and halogen-substituted sugar moieties comprising one of the following at the 2′ position: OH, SH, SCH₃, F, OCN, OCH₃OCH₃, OCH₃—O—(CH₂)ₙCH₃, O(CH₂)ₙNH₂ or O(CH₂)ₙCH₃, where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a cholesteryl group; a folate group; a reporter group; an intercalator; a group for improving the pharmacokinetic properties of an oligonucleotide; or a group for improving the pharmacodynamic properties of an oligonucleotide and other substituents having similar properties. Folate, cholesterol or other groups that facilitate oligonucleotide uptake, such as lipid analogs, may be conjugated directly or via a linker at the 2′ position of any nucleoside or at the 3′ or 5′ position of the 3′-terminal or 5′-terminal nucleoside, respectively. One or more such conjugates may be used. Oligonucleotides may also have sugar mimetics such as cyclobutyls in place of the pentofuranosyl group. Other embodiments may include at least one modified base form or “universal base” such as inosine, or inclusion of other nonstandard bases such as queosine and wybutosine as well as acetyl-, methyl-, thio- and similarly modified forms of adenine, cytidine, guanine, thymine, and uridine which are not as easily recognized by endogenous endonucleases. The antisense oligonucleotide can comprise at least one modified base moiety which is selected from the group including, but not limited to, 5 fluorouracil, 5 bromouracil, 5 chlorouracil, 5 iodouracil, hypoxanthine, xanthine, 4 acetylcytosine, 5 (carboxyhydroxylmethyl) uracil, 5 carboxymethylaminomethyl-2 thiouridine, 5 carboxymethylaminomethyluracil, dihydrouracil, beta D-galactosylqueosine, inosine, N6 isopentenyladenine, 1 methylguanine, 1 methylinosine, 2,2 dimethylguanine, 2 methyladenine, 2 methylguanine, 3 methylcytosine, 5 methylcytosine, N6 adenine, 7 methylguanine, 5 methylaminomethyluracil, 5 methoxyaminomethyl-2 thiouracil, beta-D mannosylqueosine, 5 D methoxycarboxymethyluracil, 5 methoxyuracil, 2 methylthio N6-isopentenyladenine, uracil 5-oxyacetic acid (v), wybutoxosine, pseudouracil, queosine, 2 thiocytosine, 5 methyl-2 thiouracil, 2 thiouracil, 4 thiouracil, 5 methyluracil, uracil-5 oxyacetic acid methylester, uracil-5 oxyacetic acid (v), 5 methyl-2 thiouracil, 3 (3 amino-3 N 2-carboxypropyl) uracil, (acp3)w, and 2,6 diaminopurine.

The invention further provides oligonucleotides having backbone analogues such as phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, chiral-methyl phosphonates, nucleotides with short chain alkyl or cycloalkyl intersugar linkages, short chain heteroatomic or heterocyclic intersugar (“backbone”) linkages, or CH₂—NH—O—CH₂, CH₂—N(CH₃)—OCH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂), or mixtures of the same. Also useful are oligonucleotides having morpholino backbone structures (U.S. Pat. No. 5,034,506).

Useful references include Oligonucleotides and Analogues, A Practical Approach, edited by F. Eckstein, IRL Press at Oxford University Press, 1991; Antisense Strategies, Annals of the New York Academy of Sciences, Volume 600, Eds. Baserga and Denhardt (NYAS1992); Milligan, et al., J. Med. Chem., 36: 1923-1937, 1993; ANTISENSE RESEARCH AND APPLICATIONS (1993, CRC Press), in its entirety and specifically Chapter 15, by Sanghvi, entitled “Heterocyclic base modifications in nucleic acids and their applications in antisense oligonucleotides;” and Antisense Therapeutics, ed. Sudhir Agrawal (Humana Press, Totowa, N.J., 1996).

In one embodiment, the antisense sequence is complementary to relatively accessible sequences of the PEDF-R mRNA (e.g., relatively devoid of secondary structure). This can be determined by analyzing predicted RNA secondary structures using, for example, the MFOLD program (Genetics Computer Group, Madison Wis.) and testing in vitro or in vivo as is known in the art. Another useful method for identifying effective antisense compositions uses combinatorial arrays of oligonucleotides (see, e.g., Milner, et al., Nature Biotechnology, 15: 537, 1997).

In some embodiments, administration of antisense oligonucleotides will result in reduction of human PEDF-R mRNA expression by at least about 50%, as assessed by Northern analysis after administration of an antisense phosphorothioate oligonucleotide at a concentration of 1 μM, 5 μM, 10 μM or 20 μM.

The invention also provides an antisense polynucleotide that has sequences in addition to the antisense sequence (i.e., in addition to anti-PEDF-R-sense sequence). In this case, the antisense sequence is contained within a polynucleotide of longer sequence. In another embodiment, the sequence of the polynucleotide consists essentially of, or is, the antisense sequence.

The antisense nucleic acids (DNA, RNA, modified, analogues, and the like) can be made using any suitable method for producing a nucleic acid, such as the chemical synthesis and recombinant methods disclosed herein. In one embodiment, for example, antisense RNA molecules of the invention can be prepared by de novo chemical synthesis or by cloning. For example, an antisense RNA that hybridizes to PEDF-R mRNA can be made by inserting (ligating) a PEDF-R DNA sequence (e.g., SEQUENCE ID No: 1, or fragment thereof) in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention. The term “operably linked” refers to a functional linkage between a nucleic acid expression control sequence (such as a promoter or enhancer) and a second nucleic acid sequence, wherein the expression control sequence directs transcription of the nucleic acid corresponding to the second sequence.

In one embodiment, antisense DNA oligodeoxyribonucleotides derived from the translation initiation site, e.g., between −10 and +10 regions of a PEDF-R nucleotide sequence, are used. For general methods relating to antisense polynucleotides, see ANTISENSE RNA AND DNA, 1988, D. A. Melton, Ed., Cold Spring Harbor Laboratory, Cold Spring Harbor, N.Y.). See also, Dagle, et al., Nucleic Acids Research, 19: 1805, 1991. For a review of antisense therapy, see, e.g., Uhlmann, et al., Chem. Reviews, 90: 543-584, 1990.

Ribozymes are enzymatic RNA molecules capable of catalyzing the specific cleavage of RNA. The mechanism of ribosome action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of PEDF-R RNA sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site can be evaluated for predicted structural features such as secondary structure that can render the oligonucleotide sequence unsuitable. The suitability of candidate targets can also be evaluated by testing their accessibility to hybridization with complementary oligonucleotides, using ribonuclease protection assays.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, Anticancer Drug Des., 6: 569-584, 1991; Helene, et al., Ann. N.Y. Acad. Sci., 660: 27-36, 1992; Maher, Bioassays, 14: 807-815,1992).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleotide sequences can be pyrimidine-based, which will result in TAT and CGC+ triplets across the three associated strands of the resulting triple helix. The pyrimidine-rich molecules provide base complementarily to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

The anti-sense RNA and DNA molecules, ribozymes and triple helix molecules of the invention can be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules can be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

Another method of suppression is sense suppression. Introduction of vectors in which a nucleic acid is configured in the sense orientation with respect to the promoter has been shown to be an effective means by which to block the transcription of target genes. For an example of the use of this method to modulate expression of endogenous genes.

For sense suppression, the introduced sequence in the vector, needing less than absolute identity, also need not be full length, relative to either the primary transcription product or fully processed mRNA. Furthermore, the introduced sequence need not have the same intron or exon pattern, and identity of non-coding segments will be equally effective. Normally, a sequence of the size ranges noted above for antisense regulation is used.

One of skill in the art will recognize that using technology based on specific nucleotide sequences (e.g., antisense or sense suppression technology), families of homologous genes can be suppressed with a single sense or antisense transcript. For instance, if a sense or antisense transcript is designed to have a sequence that is conserved among a family of genes, then multiple members of a gene family can be suppressed. Conversely, if the goal is to only suppress one member of a homologous gene family, then the sense or antisense transcript should be targeted to sequences with the most variance between family members.

Another means of inhibiting gene function is by creation of dominant negative mutations. In this approach, non-functional, mutant polypeptides of the present invention, which retain the ability to interact with wild-type subunits are introduced.

Expression of a polypeptide of the present invention may also be specifically suppressed by methods such as RNA interference (RNAi). A review of this technique is found in Science, 288: 1370-1372, 2000, herein incorporated by reference in its entirety for all purposes. Briefly, traditional methods of gene suppression, employing anti-sense RNA or DNA, operate by binding to the reverse sequence of a gene of interest such that binding interferes with subsequent cellular processes and therefore blocks synthesis of the corresponding protein. RNAi also operates on a post-translational level and is sequence specific, but suppresses gene expression far more efficiently. Exemplary methods for controlling or modifying gene expression are provided in WO 99/49029, WO 99/53050 and WO0/75164, the disclosures of which are hereby incorporated by reference in their entirety for all purposes. In these methods, post-transcriptional gene silencing is brought about by a sequence-specific RNA degradation process which results in the rapid degradation of transcripts of sequence-related genes. Studies have shown that double-stranded RNA may act as a mediator of sequence-specific gene silencing (see, for example, Montgomery, et al., Trends in Genetics, 14: 255-258, 1998). Gene constructs that produce transcripts with self-complementary regions are particularly efficient at gene silencing.

It has been demonstrated that one or more ribonucleases specifically bind to and cleave double-stranded RNA into short fragments. The ribonuclease(s) remains associated with these fragments, which in turn specifically bind to complementary mRNA, i.e. specifically bind to the transcribed mRNA strand for the gene of interest. The mRNA for the gene is also degraded by the ribonuclease(s) into short fragments; thereby obviating translation and expression of the gene. Additionally, an RNA-polymerase may act to facilitate the synthesis of numerous copies of the short fragments, which exponentially increases the efficiency of the system. A unique feature of RNAi is that silencing is not limited to the cells where it is initiated. The gene-silencing effects may be disseminated to other parts of an organism.

The polynucleotides of the present invention may thus be employed to generate gene silencing constructs and/or gene-specific self-complementary, double-stranded RNA sequences that can be delivered by conventional art-known methods. A gene construct may be employed to express the self-complementary RNA sequences. Alternatively, cells are contacted with gene-specific double-stranded RNA molecules, such that the RNA molecules are internalized into the cell cytoplasm to exert a gene silencing effect. The double-stranded RNA must have sufficient homology to the targeted gene to mediate RNAi without affecting expression of non-target genes. The double-stranded DNA is at least 20 nucleotides in length, and is preferably 21-23 nucleotides in length. Preferably, the double-stranded RNA corresponds specifically to a polynucleotide of the present invention. The use of small interfering RNA (siRNA) molecules of 21-23 nucleotides in length to suppress gene expression in mammalian cells is described in WO 01/75164. Tools for designing optimal inhibitory siRNAs include that available from DNAengine Inc. (Seattle, Wash.). See WO 01/68836. See also: Bernstein, et al., RNA, 7: 1509-1521, 2001; Bernstein, et al., Nature, 409: 363-366, 2001; Billy, et al, Proc. Nat'l Acad. Sci. U.S.A., 98: 14428-33, 2001; Caplan, et al., Proc. Nat'l Acad. Sci. U.S.A., 98: 9742-7, 2001; Carthew, et al., Curr. Opin. Cell Biol., 13: 244-8,2001; Elbashir, et al., Nature, 411: 494-498, 2001; Hammond, et al., Science, 293: 1146-50, 2001; Hammond, et al., Nat. Ref. Genet., 2: 110-119, 2001; Hammond, et al., Nature, 404: 293-296,2000; McCaffrrey, et al., Nature, 418-438-439, 2002; McCaffrey, et al., Mol. Ther., 5: 676-684, 2002; Paddison, et al., Genes Dev., 16: 948-958, 2002; Paddison, et al., Proc. Nat'l Acad. Sci. U.S.A., 99:1443-1448, 2002; Sui, et al., Proc. Nat'l Acad. Sci. U.S.A., 99: 5515-5520, 2002. U.S. Patents of interest include U.S. Pat. Nos. 5,985,847 and 5,922,687. Also of interest is WO/11092. Additional references of interest include: Acsadi, et al., New Biol., 3: 71-81, 1991; Chang, et al., J. Virol., 75: 3469-3473, 2001; Hickman, et al., Hum. Gen. Ther., 5: 1477-1483, 1994; Liu, et al., Gene Ther., 6: 1258-1266, 1999; Wolff, et al., Science, 247: 1465-1468, 1990; Zhang, et al., Hum. Gene Ther., 10: 1735-1737, 1999; Zhang, et al., Gene Ther., 7: 1344-1349, 1999. These disclosures are herein incorporated by reference in their entirety for all purposes.

In another aspect is provided the administration of PEDF-R to a mammal having depressed levels of endogenous PEDF-R or a defective PEDF-R gene, preferably in the situation where such depressed levels lead to a pathological disorder, or where there is lack of activation of the PEDF-R. In these embodiments where the full length PEDF-R is to be administered to the patient, it is contemplated that the gene encoding the receptor can be administered to the patient via gene therapy technology.

In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik, et al., Proc. Natl. Acad. Sci. U.S.A., 83: 4143-4146, 1986). The oligonucleotides can be modified to enhance their uptake, e.g., by substituting their negatively charged phosphodiester groups by uncharged groups.

There are a variety of techniques available for introducing nucleic acids into viable cells. The techniques vary depending upon whether the nucleic acid is transferred into cultured cells in vitro, ex vivo, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral vectors and viral coat proteinliposome mediated transfection (Dzau, et al., Trends in Biotechnology, 11: 205-210, 1993). Viral vector mediated techniques may employ a variety of viruses in the construction of the construct for delivering the gene of interest. The type of viral vector used is dependent on a number of factors including immunogenicity and tissue tropism. Some non-limiting examples of viral vectors useful in gene therapy include retroviral vectors (see e.g., U.S. Pat. Nos. 6,312,682, 6,235,522, 5,672,510 and 5,952,225,), adenoviral (Ad) vectors (see e.g., U.S. Pat. Nos. 6,482,616, 5,846,945) and adeno-associated virus (AAV) vectors (see, e.g., U.S. Pat. Nos. 6,566,119, 6,392,858, 6,468,524 and WO 99/61601). In some situations it is desirable to provide the nucleic acid source with an agent that targets the target cells, such as an antibody specific for a cell surface membrane protein or the target cell, a ligand for a receptor on the target cell, and the like. Where liposomes are employed, proteins which bind to a cell surface membrane protein associated with endocytosis can be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu, et al., J. Biol. Chem., 262: 4429-4432, 1987; Wagner, et al., Proc. Natl. Acad. Sci. U.S.A., 87: 3410-3414, 1990. For review of the currently known gene marking and gene therapy protocols see Anderson, et al., Science, 256: 808-813, 1992.

The invention also provides antagonists of PEDF-R activation (e.g., PEDF-R antisense nucleic acid, neutralizing antibodies). Administration of PEDF-R antagonist to a mammal having increased or excessive levels of endogenous PEDF-R activation is contemplated, preferably in the situation where such increased levels of PEDF-R lead to a pathological disorder.

In one embodiment, PEDF-R antagonist molecules can be used to bind endogenous ligand in the body, thereby causing desensitized PEDF-R to become responsive to PEDF ligand, especially when the levels of PEDF ligand in the serum exceed normal physiological levels. Also, it can be beneficial to bind endogenous PEDF ligand which is activating undesired cellular responses (such as proliferation of tumor cells).

In numerous embodiments of this invention, a compound, e.g., nucleic acid, polypeptide, or other molecule is administered to a patient, in vivo or ex vivo, to effect a change in PEDF-R activity or expression in the patient. The desired change can be either an increase or a decrease in activity or expression of PEDF-R activity. For example, in a cancer patient with a tumor that exhibits decreased levels of PEDF-R relative to normal tissue, it can be desirable to increase the activity or expression of PEDF-R. In other embodiments of the invention, antibodies that block PEDF activity or function can be administered to a patient, for example, neovascularization and wound healing.

Compounds that can be administered to a patient include nucleic acids encoding full length PEDF polypeptides, or any derivative, fragment, or variant thereof, operably linked to a promoter. Suitable nucleic acids also include inhibitory sequences such as antisense or ribozyme sequences, which can be delivered in, e.g., an expression vector operably linked to a promoter, or can be delivered directly. Also, any nucleic acid that encodes a polypeptide that modulates the expression of PEDF-R can be used. In general, nucleic acids can be delivered to cells using any of a large number of vectors or methods, e.g., retroviral, adenoviral, or adeno-associated virus vectors, liposomal formulations, naked DNA injection, and others. All of these methods are well known to those of skill in the art.

Proteins can also be delivered to a patient to modulate PEDF-R activity. In preferred embodiments, a polyclonal or monoclonal antibody that specifically binds to PEDF will be delivered. In addition, any polypeptide that interacts with and/or modulates PEDF-R can be used, e.g., a polypeptide that is identified using the presently described assays. In addition, polypeptides that affect PEDF expression can be used.

Further, any compound that is found to or designed to interact with and/or modulate the activity of PEDF-R can be used. For example, any compound that is found, using the methods described herein, to bind to or modulate the activity of PEDF-R can be used.

Any of the above-described molecules can be used to increase or decrease the expression or activity of PEDF-R, or to otherwise affect the properties and/or behavior of PEDF-R polypeptides or polynucleotides, e.g., stability, intracellular localization, interactions with other intracellular or extracellular moieties, and the like.

The invention provides pharmaceutical compositions comprising one or a combination of PEDF-R modulators formulated together with a pharmaceutically acceptable carrier.

Dosage regimens of the pharmaceutical compositions of the present invention are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the compounds of the invention employed in the pharmaceutical composition at levels lower than that required to achieve the desired therapeutic effect and gradually increase the dosage until the desired effect is achieved. In general, a suitable daily dose of a compound of the invention is that amount of the compound which is the lowest dose effective to produce a therapeutic effect. Such an effective dose generally depends upon the factors described above. It is preferred that administration be intravenous, intramuscular, intraperitoneal, or subcutaneous, or administered proximal to the site of the target. If desired, the effective daily dose of a therapeutic composition can be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody to PEDF-R in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for nucleic acids encoding immunogens range from about 10 ng to 1 g, 100 ng to 100 mg, 1 μg to 10 mg, or 30-300 μg DNA per patient. Doses for infectious viral vectors vary from 10-100, or more, virions per dose.

Some compounds of the invention can be formulated to ensure proper distribution in vivo. For example, the blood-brain barrier (BBB) excludes many highly hydrophilic compounds. To ensure that the therapeutic compounds of the invention cross the BBB. (if desired), they can be formulated, for example, in liposomes. For methods of manufacturing liposomes, See, e.g., U.S. Pat. Nos. 4,522,811; 5,374,548; and 5,399,331. The liposomes can comprise one or more moieties which are selectively transported into specific cells or organs, thus enhance targeted drug delivery (See, e.g., Ranade, J Clin. Pharmacol., 29: 685, 1989). Exemplary targeting moieties include folate or biotin (See, e.g., U.S. Pat. No. 5,416,016 to Low, et al.); mannosides (Umezawa, et al., Biochem. Biophys. Res. Commun., 153: 1038, 1988); antibodies (Bloeman, et al., FEBS Lett., 357: 140, 1995; Owais, et al., Antimicrob. Agents Chemother., 39: 180, 1995); surfactant protein A receptor (Briscoe, et al., Am. J. Physiol., 1233: 134, 1995), different species of which can comprise the formulations of the inventions, as well as components of the invented molecules; p120 (Schreier, et al., J. Biol. Chem., 269: 9090, 1994); See also Keinanen, et al., FEBS Lett., 346: 123, 1994; Killion, et al., Immunomethods, 4: 273, 1994. In some methods, the therapeutic compounds of the invention are formulated in liposomes; in a more preferred embodiment, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

The present invention also includes a device for preventing or treating nerve damage or damage to other cells as taught herein by implantation into a patient a construct comprising a semipermeable membrane, and a cell that secretes PEDF-R (or its agonists or antagonists as can be required for the particular condition) encapsulated within said membrane and said membrane being permeable to PEDF-R (or its agonists or antagonists) and impermeable to factors from the patient detrimental to the cells. The patient's own cells, transformed to produce PEDF-R or a PEDF-R ligand ex vivo, could be implanted directly into the patient, optionally without such encapsulation. The methodology for the membrane encapsulation of living cells is familiar to those of ordinary skill in the art, and the preparation of the encapsulated cells and their implantation in patients can be accomplished without under experimentation.

The present invention includes, therefore, a method for preventing or treating nerve damage by implanting cells, into the body of a patient in need thereof, cells either selected for their natural ability to generate PEDF-R or a PEDF-R ligand or engineered to secrete PEDF-R or a PEDF-R ligand. The implants are preferably non-immunogenic and/or prevent immunogenic implanted cells from being recognized by the immune system. For CNS delivery, a preferred location for the implant is the cerebral spinal fluid of the spinal cord.

For therapeutic applications, the pharmaceutical compositions are administered to a patient suffering from established disease in an amount sufficient to arrest or inhibit further development or reverse or eliminate, the disease, its symptoms or biochemical markers. For prophylactic applications, the pharmaceutical compositions are administered to a patient susceptible or at risk of a disease in an amount sufficient to delay, inhibit or prevent development of the disease, its symptoms and biochemical markers. An amount adequate to accomplish this is defined as a “therapeutically-” or “prophylactically-effective dose.” Dosage depends on the disease being treated, the subject's size, the severity of the subject's symptoms, and the particular composition or route of administration selected. Specifically, in treatment of tumors, a “therapeutically effective dosage” can inhibit tumor growth by at least about 20%, or at least about 40%, or at least about 60%, or at least about 80% relative to untreated subjects. The ability of a compound to inhibit cancer can be evaluated in an animal model system predictive of efficacy in human tumors. Alternatively, this property of a composition can be evaluated by examining the ability of the compound to inhibit by conventional assays in vitro. A therapeutically effective amount of a therapeutic compound can decrease tumor size, or otherwise ameliorate symptoms in a subject.

The pharmaceutical composition of the present invention should be sterile and fluid to the extent that the composition is deliverable by syringe. In addition to water, the carrier can be an isotonic buffered saline solution, ethanol, polyol (for example, glycerol, propylene glycol, and liquid polyethylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound can be orally administered, for example, with an inert diluent or an assimilable edible carrier.

Pharmaceutical compositions of the invention also can be administered in combination therapy, i.e., combined with other agents. For example, in treatment of cancer, the combination therapy can include a composition of the present invention with at least one anti-tumor agent or other conventional therapy, such as radiation treatment. Likewise in treatment of neurological or ocular disorders, e.g., retinitis pigmentosa, the combination therapy can include a composition of the present invention with at least one agent useful for treating neurological or ocular disorders.

Pharmaceutically acceptable carriers include solvents, dispersion media, coatings, antibacterial and antifungal agents, isotonic and absorption delaying agents, and the like that are physiologically compatible. The carrier can be suitable for intravenous, intraocular, intramuscular, subcutaneous, parenteral, spinal or epidermal administration (e.g., by injection or infusion). Depending on the route of administration, the active compound, i.e., antibody, bispecific and multispecific molecule, can be coated in a material to protect the compound from the action of acids and other natural conditions that can inactivate the compound.

A “pharmaceutically acceptable salt” refers to a salt that retains the desired biological activity of the parent compound and does not impart any undesired toxicological effects (See, e.g., Berge, et al., J. Pharm. Sci., 66: 1-19, 1977). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., SUSTAINED AND CONTROLLED RELEASE DRUG DELIVERY SYSTEMS, J. R. Robinson, Ed., 1978, Marcel Dekker, Inc., New York.

To administer a compound of the invention by certain routes of administration, it can be necessary to coat the compound with, or co-administer the compound with, a material to prevent its inactivation. For example, the compound can be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan, et al., J Neuroimmunol, 7: 27, 1984).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions can also be administered with medical devices known in the art. For example, in a preferred embodiment, a therapeutic composition of the invention can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

When the compounds of the present invention are administered as pharmaceuticals, to humans and animals, they can be given alone or as a pharmaceutical composition containing, for example, 0.01 to 99.5% (or 0.1 to 90%) of active ingredient in combination with a pharmaceutically acceptable carrier.

The pharmaceutical compositions are generally formulated as sterile, substantially isotonic and in full compliance with all Good Manufacturing Practice (GMP) regulations of the U.S. Food and Drug Administration.

The present invention provides methods of administering compounds to the eye to treat ocular disease. Thus, exemplary compositions according to the present invention are suitable for direct administration to a subject's eye. By “direct administration” it is meant that the compositions are applied topically or by injection or installation into the eye. There are a wide variety of suitable formulations of pharmaceutical compositions of the present invention (See e.g., Remington's PHARMACEUTICAL SCIENCES, 19th ed., 1995).

The compositions of the present invention can be compounded with one or more agents to facilitate their use in a wide variety of contexts. Topical compositions for delivering compounds s to the eye according to the present invention will typically comprise the compound present in a suitable ophthalmically acceptable carrier. Exemplary ophthalmically acceptable-carriers include, but are not limited to, water, buffered aqueous solutions, isotonic mixtures of water and water-immiscible solvents, such as alkanols, aryl alkanols, vegetable oils, polyalkalene glycols, petroleum-based jellies, ethylcellulose, ethyloleate, carboxymethylcelluloses, polyvinylpyrrolidones, and isopropyl myristates. The compositions of the present invention can also include ophthahmically acceptable auxiliary components such as buffers, emulsifiers, preservatives, wetting agents, tonicity agents, thixotropic agents, e.g., polyethylene glycols, chelating agents, and additional antimicrobial agents.

The agents of the present invention are incorporated into suitable ophthalmically acceptable carriers at therapeutically effective concentrations. For treatment purposes, the pharmaceutical formulations of the present invention can be administered to the subject in a single bolus delivery, via continuous delivery over an extended time period, or in a repeated administration protocol (e.g., by an hourly, daily or weekly, repeated administration protocol). The pharmaceutical formulations of the present invention can be administered, for example, one or more times half-hourly, i.e., every half an hour for a 24 hour period, one or more times hourly, or one or more times daily. In certain embodiments, the pharmaceutical formulations of the invention are administered two times daily, four times daily, six times daily, or twelve times daily. Typically, the formulations are self-administered

Topical administration according to the present invention also includes the application of ophthalmic ointments and gels containing one or more compound of the present invention to the eye. The ophthalmic ointments can include any substances known to the skilled formulation chemist to be useful for the preparation of such ointments. Typically, the ophthalmic ointments will include a base which permits diffusion of the drug into the ocular fluid. In exemplary embodiments of the present invention, the base will be comprised of white petrolatum and mineral oil and other substances known in the art as being appropriate for administration to the eye, e.g., anhydrous lanolin and/or polyethylene-mineral oil gel. The amount of a compound of the present invention in the ointment or gel can vary widely depending on the type of composition, size of a unit dosage, kind of excipients, and other factors well known to those of ordinary skill in the art. In general, the final composition can comprise from 0.000001 percent by weight (wt %) to 10 wt % of the compound, preferably 0.00001 wt % to 1 wt %, with the remainder being the excipient or excipients.

The PEDF-R can be used for competitive screening of potential agonists or antagonists for binding to the PEDF-R. Such agonists or antagonists can constitute potential therapeutics for treating conditions characterized by insufficient or excessive PEDF-R activation, respectively.

A preferred technique for identifying molecules which bind to the PEDF-R utilizes a chimeric receptor (e.g., epitope-tagged PEDF-R or PEDF-R immunoadhesin) attached to a solid phase, such as the well of an assay plate. The binding of the candidate molecules, which are optionally labelled (e.g., radiolabeled), to the immobilized receptor can be measured. Alternatively, competition for binding of a known, labelled PEDF-R ligand, such as ¹²⁵I PEDF, can be measured. For screening for antagonists, the PEDF-R can be exposed to a PEDF ligand followed by the putative antagonist, or the PEDF ligand and antagonist can be added to the PEDF-R simultaneously, and the ability of the antagonist to block receptor activation can be evaluated.

The present invention also provides for assay systems for detecting PEDF activity, comprising cells which express high levels of PEDF-R, and which are, therefore, extremely sensitive to even very low concentrations of PEDF or PEDF-like molecules. The present invention provides for assay systems in which PEDF activity or activities similar to PEDF activity resulting from exposure to a peptide or non-peptide compound can be detected by measuring a physiological response to PEDF in a cell or cell line responsive to PEDF which expresses the PEDF-R molecules of the invention. A physiological response can comprise any of the biological effects of PEDF, including but not limited to, those described herein, as well as the transcriptional activation of certain nucleic acid sequences (e.g. promoter/enhancer elements as well as structural genes), PEDF-related processing, translation, or phosphorylation, the induction of secondary processes in response to processes directly or indirectly induced by PEDF, including morphological changes, such as neurite sprouting, or the ability to support the survival of cells, for example, retinal cells, cerebellar granle neurons, nodose or dorsal root ganglion cells, motoneurons, dopaminergic neurons, sensory, neurons, Purkinje cells, or hippocampal neurons.

The present invention provides for the development of novel assay systems which can be utilized in the screening of compounds for PEDF- or PEDF-like activity. Target cells which bind PEDF can be produced by transfection with PEDF-R-encoding nucleic acid or can be identified and segregated by, for example, fluorescent-activated cell sorting, sedimentation of rosettes, or limiting dilution. Once target cell lines are produced or identified, it can be desirable to select for cells which are exceptionally sensitive to PEDF. Such target cells can bear a greater number of PEDF-R molecules; target cells bearing a relative abundance of PEDF-R can be identified by selecting target cells which bind to high levels of PEDF, for example, by marking high-expressors with fluorophore tagged-PEDF followed by immunofluorescence detection and cell sorting. Alternatively, cells which are exceptionally sensitive to PEDF can exhibit a relatively strong biological response in response to PEDF binding. By developing assay systems using target cells which are extremely sensitive to PEDF, the present invention provides for methods of screening for PEDF or PEDF-like activity which are capable of detecting low levels of PEDF activity.

In particular, using recombinant DNA techniques, the present invention provides for PEDF-R target cells which are engineered to be highly sensitive to PEDF. For example, the PEDF-receptor gene can be inserted into cells which are naturally PEDF responsive such that the recombinant PEDF-R gene is expressed at high levels and the resulting engineered target cells express a high number of PEDF-Rs on their cell surface. Cells that overexpress the PEDF-R are useful, for example, in studies on signal transduction pathways mediated specifically by PEDF. In some embodiments, the target cells can be engineered to comprise a recombinant gene which is expressed at high levels in response to PEDF/receptor binding. Such a recombinant gene can preferably be associated with a readily detectable product. For example, and not by way of limitation, transcriptional control regions (i.e. promoter/enhancer regions) from an immediate early gene can be used to control the expression of a reporter gene in a construct which can be introduced into target cells. The immediate early gene/reporter gene construct, when expressed at high levels in target cells by virtue of a strong promoter/enhancer or high copy number, can be used to produce an amplified response to PEDF-R binding. For example, and not by way of limitation, a PEDF-responsive promoter can be used to control the expression of detectable reporter genes including β-galactosidase, growth hormone, chloramphenicol acetyl transferase, neomycin phosphotransferase, luciferase, or P-glucuronidase. Detection of the products of these reporter genes, well known to one skilled in the art, can serve as a sensitive indicator for PEDF or PEDF-like activity of pharmaceutical compounds. The PEDF-R encoding or reporter gene constructs discussed herein can be inserted into target cells using any method known in the art, including but not limited to transfection, electroporation, calcium phosphate/DEAE dextran methods, and cell gun. The constructs and engineered target-cells can be used for the production of transgenic animals bearing the above-mentioned constructs as transgenes, from which PEDF-R expressing target cells can be selected using the methods discussed.

Nucleic acids which encode PEDF-R, preferably from non-human species, such as murine or rat protein, can be used to generate either transgenic animals or “knock out” animals which, in turn, are useful in the development and screening of therapeutically useful reagents. A transgenic animal (e.g., a mouse) is an animal having cells that contain a transgene, which transgene was introduced into the animal or an ancestor of the animal at a prenatal, e.g., an embryonic, stage. A transgene is a DNA which is integrated into the genome of a cell from which a transgenic animal develops. In one embodiment, the human and/or rat cDNA encoding PEDF-R, or an appropriate sequence thereof, can be used to clone genomic DNA encoding PEDF-R in accordance with established techniques and the genomic sequences used to generate transgenic animals that contain cells which express DNA encoding PEDF-R. Methods for generating transgenic animals, particularly animals such as mice, have become conventional in the art and are described, for example, in U.S. Pat. Nos. 4,736,866 and 4,870,009. Typically, particular cells would be targeted for PEDF-R transgene incorporation with tissue-specific enhancers, which could result in desired effect of treatment. Transgenic animals that include a copy of a transgene encoding PEDF-R introduced into the germ line of the animal at an embryonic stage can be used to examine the effect of increased expression of DNA encoding PEDF-R. Such animals can be used as tester animals for reagents thought to confer protection from, for example, diseases related to PEDF. In accordance with this facet of the invention, an animal is treated with the reagent and a reduced incidence of the disease, compared to untreated animals bearing the transgene, would indicate a potential therapeutic intervention for the disease.

Transgenic mice bearing minigenes are currently preferred. First a fusion enzyme expression construct is created and selected based on expression in cell culture as described in the Examples. Then a minigene capable of expressing that fusion enzyme is constructed using known techniques. Particularly preferred hosts are those bearing minigene constructs comprising a transcriptional regulatory element that is tissue-specific for expression.

Transgenic mice expressing PEDF-R minigene are made using known techniques, involving, for example, retrieval of fertilized ova, microinjection of the DNA construct into male pronuclei, and re-insertion of the fertilized transgenic ova into the uteri of hormonally manipulated pseudopregnant foster mothers. Alternatively, chimeras are made using known techniques employing, for example, embryonic stem cells (Rossant, et al., Philos. Trans. R. Soc. Lond. Biol., 339: 207-215, 1993) or primordial germ cells (Vick, et al. Philos. Trans. R. Soc. Lond. Biol., 251: 179-182, 1993) of the host species. Insertion of the transgene can be evaluated by Southern blotting of DNA prepared from the tails of offspring mice. Such transgenic mice are then back-crossed to yield homozygotes.

It is now well-established that transgenes are expressed more efficiently if they contain introns at the 5′ end, and if these are the naturally occurring introns (Brinster, et al. Proc. Natl. Acad. Sci. U.S.A., 85: 836, 1988; Yokode, et al., Science, 250: 1273, 1990).

Transgenic mice expressing PEDF-R minigene are created using established procedures for creating transgenic mice. Transgenic mice are constructed using now standard methods (Brinster, et al., Proc. Natl. Acad. Sci. U.S.A., 85: 836, 1988; Yokode, et al., Science, 250: 1273, 1990; Rubin, et al., Proc. Nat'l Acad. Sci. U.S.A., 88: 434, 1991; Rubin, et al., Nature, 353: 265, 1991). Fertilized eggs from timed matings are harvested from the oviduct by gentle rinsing with PBS and are microinjected with up to 100 nanoliters of a DNA solution, delivering about 104 DNA molecules into the male pronucleus. Successfully injected eggs are then re-implanted into pseudopregnant foster mothers by oviduct transfer. Less than 5% of microinjected eggs yield transgenic offspring and only about ⅓ of these actively express the transgene: this number is presumably influenced by the site at which the transgene enters the genome.

Transgenic offspring are identified by demonstrating incorporation of the microinjected transgene into their genomes, preferably by preparing DNA from short sections of tail and analyzing by Southern blotting for presence of the transgene (“Tail Blots”). A preferred probe is a segment of a minigene fusion construct that is uniquely present in the transgene and not in the mouse genome. Alternatively, substitution of a natural sequence of codons in the transgene with a different sequence that still encodes the same peptide yields a unique region identifiable in DNA and RNA analysis. Transgenic “founder” mice identified in this fashion are bred with normal mice to yield heterozygotes, which are back-crossed to create a line of transgenic mice. Tail blots of each mouse from each generation are examined until the strain is established and homozygous. Each successfully created founder mouse and its strain vary from other strains in the location and copy number of transgenes inserted into the mouse genome, and hence have widely varying levels of transgene expression. Selected animals from each established line are sacrificed at 2 months of age and the expression of the transgene is analyzed by Northern blotting of RNA from liver, muscle, fat, kidney, brain, lung, heart, spleen, gonad, adrenal and intestine.

Alternatively, the non-human homologs of PEDF-R can be used to construct a PEDF-R “knock out” animal, i.e., having a defective or altered gene encoding PEDF-R, as a result of homologous recombination between the endogenous PEDF-R gene and an altered genomic PEDF-R DNA introduced into an embryonic cell of the animal. For example, murine PEDF-R cDNA can be used to clone genomic PEDF-R DNA in accordance with established techniques. A portion of the genomic PEDF-R DNA (e.g., such as an exon which encodes e.g., an extracellular domain) can be deleted or replaced with another gene, such as a gene encoding a selectable marker which can be used to monitor integration. Typically, several kilobases of unaltered flanking DNA (both at the 5′ and 3′ ends) are included in the vector (see e.g., Thomas and Capecchi, Cell, 51: 503, 1987, for a description of homologous recombination vectors). The vector is introduced into an embryonic stem cell line (e.g. by electroporation) and cells in which the introduced DNA has homologously recombined with the endogenous DNA are selected (see e.g., Li, et al., Cell, 69: 915, 1992). The selected cells are then injected into a blastocyst of an animal (e.g., a mouse) to form aggregation chimeras (see e.g., Bradley, in Teratocarcinomas And Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for their ability to accept grafts, reject tumors and defend against infectious diseases and can be used in the study of basic immunobiology.

In addition to the above procedures, which can be used for preparing recombinant DNA molecules and transformed host animals in accordance with the practices of this invention, other known techniques and modifications thereof can be used in carrying out the practice of the invention. For example, U.S. Pat. No. 4,736,866 discloses vectors and methods for production of a transgenic non-human eukaryotic animal-whose germ cells and somatic cells contain a gene sequence introduced into the animal, or an ancestor of the animal, at an embryonic stage. U.S. Pat. No. 5,087,571 discloses a method of providing a cell culture comprising (1) providing a transgenic non-human mammal, all of whose germ cells and somatic cells contain a recombinant gene sequence introduced at an embryonic stage; and (2) culturing one or more of said somatic cells. U.S. Pat. No. 5,175,385 discloses vectors and methods for production of a transgenic mouse whose somatic and germ cells contain and express a gene at sufficient levels to provide the desired phenotype in the mouse, the gene having been introduced into said mouse or an ancestor of said mouse at an embryonic stage, preferably by microinjection. A partially constitutive promoter, the metallothionein promoter, was used to drive heterologous gene expression. U.S. Pat. No. 5,175,384 discloses a method of introducing a transgene into an embryo by infecting the embryo with a retrovirus containing the transgene. U.S. Pat. No. 5,175,383 discloses DNA constructs having a gene, homologous to the host cell, operably linked to a heterologous and inducible promoter effective for the expression of the gene in the urogenital tissues of a mouse, the transgene being introduced into the mouse at an embryonic stage to produce a transgenic mouse. Even though a homologous gene is introduced, the gene can integrate into a chromosome of the mouse at a site different from the location of the endogenous coding sequence. The vital MMTV promoter was disclosed as a suitable inducible promoter. U.S. Pat. No. 5,162,215 discloses methods and vectors for transfer of genes in avian species, including livestock species such as chickens, turkeys, quails or ducks, utilizing pluripotent stem cells of embryos to produce transgenic animals. U.S. Pat. No. 5,082,779 discloses pituitary-specific expression promoters for use in producing transgenic animals capable of tissue-specific expression of a gene. U.S. Pat. No. 5,075,229 discloses vectors and methods to produce transgenic, chimeric animals whose hemopoietic liver cells contain and express a functional gene driven by a liver-specific promoter, by injecting into the peritoneal cavity of a host fetus the disclosed vectors such that the vector integrates into the genome of fetal hemopoietic liver cells.

Although some of the above-mentioned patents and publications are directed to the production or use of a particular gene product or material that are not within the scope of the present invention, the procedures described therein can easily be modified to the practice of the invention described in this specification by those skilled in the art of fermentation and genetic engineering.

Assay systems of the present invention enable the efficient screening of pharmaceutical compounds for use in the treatment of PEDF-associated diseases. For example, and not by way of limitation, it can be desirable to screen a pharmaceutical agent for PEDF activity and therapeutic efficacy in, for example, cerebellar, motor, hippocampal, or retinal degeneration or angiogenesis. (J Neuropathol. Exp. Neurol., 58: 719-28, 1999; J Comp. Neurol., 412: 506-14, 1999; J. Neurosci., 22: 9378-86, 2002; J. Neurosci. Res., 56: 604-610, 1999).

In a one embodiment of the invention, cells responsive to PEDF can be identified and isolated, and then cultured in microwells in a multiwell culture plate. Culture medium with added test agent, or added PEDF, in numerous dilutions can be added to the wells, together with suitable controls. The cells can then be examined for improved survival, neurite sprouting, and the like, and the activity of test agent and PEDF, as well as their relative activities, can be determined. For example, one can now identify PEDF-like compounds which can, like PEDF, prevent retinal cell death or prolong retinal cell survival in response to toxic assault or axotomy, for example. PEDF responsive retinal neurons could be utilized in assay systems to identify compounds useful in treating retinal diseases. If a particular disease is found to be associated with a defective PEDF response in a particular tissue, a rational treatment for the disease would be supplying the patient with exogenous PEDF. However, it can be desirable to develop molecules which have a longer half-life than endogenous PEDF, or which act as PEDF agonists, or which are targeted to a particular tissue. Accordingly, the methods of the invention can be used to produce efficient and sensitive screening systems which can be used to identify molecules with the desired properties. Similar assay systems could be used to identify PEDF antagonists.

In addition, the present invention provides for experimental model systems for studying the physiological role of PEDF-R ligand and PEDF-R. Such systems include animal models, such as (i) animals exposed to circulating PEDF-R peptides which compete with cellular receptor for PEDF binding and thereby produce a PEDF-R depleted condition, (ii) animals immunized with PEDF-R; (iii) transgenic animals which express high levels of PEDF-R and therefore are hypersensitive to PEDF ligands; and (iv) animals derived using embryonic stem cell technology in which the endogenous PEDF-R genes were deleted from the genome.

The present invention also provides for experimental model systems for studying the physiological role of PEDF-R and its ligand. In these model systems, PEDF-R protein, peptide fragment, PEDF-R ligand, can be either supplied to the system or produced within the system. Such model systems could be used to study the effects of PEDF-R ligand excess or PEDF-R ligand depletion. The experimental model systems can be used to study the effects of increased or decreased response to PEDF-R ligand in cell or tissue cultures, in whole animals, in particular cells or tissues within whole animals or tissue culture systems, or over specified time intervals (including during embryogenesis) in embodiments in which PEDF-R expression is controlled by an inducible or developmentally regulated promoter. In a particular embodiment of the invention, the CMV promoter can be used to control expression of PEDF-R in transgenic animals. Transgenic animals, as discussed herein, are produced by any method known in the art, including, but not limited to microinjection, cell fusion, transfection, and electroporation.

The purified PEDF-R and the nucleic acid encoding it, can also be sold as reagents for studies of PEDF-R and its ligands, including, for example, to study the role of the PEDF-R and PEDF ligand in normal growth and development, as well as abnormal growth and development, e.g., in malignancies. PEDF-R probes can be used to identify cells and tissues which are responsive to PEDF ligand in normal or diseased states. For example, a patient suffering from a PEDF-related disorder can exhibit an aberrancy of PEDF-R expression. The present invention provides for methods for identifying cells which are responsive to PEDF-R ligand comprising detecting PEDF-R expression in such cells. PEDF-R expression can be evidenced by transcription of PEDF-R mRNA or production of PEDF-R protein. PEDF-R expression can be detected using probes which identify PEDF-R nucleic acid or protein. The purified PEDF-R and the nucleic acid encoding it, can also be sold as reagents for the identification of PEDF-R ligands.

According to the invention, tagged PEDF ligand can be incubated with cells under conditions which would promote the binding or attachment of PEDF ligand to said cells. In most cases, this can be achieved under standard culture conditions. For example, in one embodiment of the invention, cells can be incubated for about 30 minutes in the presence of tagged PEDF ligand. If the tag is an antibody molecule, it can be preferable to allow PEDF ligand to bind to cells first and subsequently wash cells to remove unbound ligand and then add anti-PEDF ligand antibody tag. In another embodiment of the invention, tagged PEDF ligand on the surface of PEDF ligand responsive cells, hereafter called target cells, can be detected by rosetting assays in which indicator cells that are capable of binding to the tag are incubated with cells bearing tagged ligand PEDF such that they adhere to tagged-PEDF ligand on the target cells and the bound indicator cells form rosette-like clusters around PEDF ligand tag bearing cells. These rosettes can be visualized by standard microscopic techniques on plated cells, or, alternatively, can allow separation of rosetted and non-rosetted cells by density centrifugation. In a preferred specific embodiment of the invention, target cells, such as neuronal cells. In alternative embodiments of the invention, tagged PEDF ligand on the surface of target cells can be detected using immunofluorescent techniques in which a molecule which reacts with the tag, preferably an antibody, directly or indirectly produces fluorescent light. The fluorescence can either be observed under a microscope or used to segregate tagged PEDF ligand bearing cells by fluorescence activated cell sorting techniques.

The present invention also provides for methods for detecting other forms of tags, such as chromogenic tags and catalytic tags. An anti-PEDF-R antibody can also be used as a probe. The detection methods for any particular tag will depend on the conditions necessary for producing a signal from the tag, but should be readily discernible by one skilled in the art.

PEDF-R variants are useful as standards or controls in assays for the PEDF-R for example ELISA, RIA, or RRA, provided that they are recognized by the analytical system employed, e.g., an anti-PEDF-R antibody.

The PEDF-R ligands of the present invention can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc.), 1987.

Competitive binding assays rely on the ability of a labeled standard to compete with the test sample analyte for binding with a limited amount of antibody. The amount of PEDF-R in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies can conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody can itself be labeled with a detectable moiety (direct sandwich assays) or can be measured using an anti-immunoglobulin antibody that is labeled with a detectable moiety (indirect sandwich assay). For example, one type of sandwich assay is an ELISA assay, in which case the detectable moiety is an enzyme.

Modulation of PEDF activity can be assessed using a variety of in vitro and in vivo assays, as described above, and, such assays can be used to test for inhibitors and activators of PEDF-R protein. Such modulators of PEDF-R protein are useful for treating disorders related to PEDF activity. Modulators of PEDF-R protein are tested using either recombinant or naturally occurring, preferably human PEDF-R

Assays to identify compounds with modulating activity can be performed in vitro. For example, PEDF-R protein is first contacted with a potential modulator and incubated for a suitable amount of time, e.g., from 0.5 to 48 hours. In one embodiment, PEDF-R polypeptide levels are determined in vitro by measuring the level of protein or mRNA. The level of PEDF-R protein or proteins related to PEDF-R signal transduction are measured using immunoassays such as western blotting, ELISA and the like with an antibody that selectively binds to the PEDF-R polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using a PEDF-R protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as green fluorescent protein (see, e.g., Mistili, et al., Nature Biotechnology, 15: 961-964, 1997). The reporter construct is typically transfected into a cell. After treatment with a potential modulator, the amount of reporter gene transcription, translation, or activity is measured according to standard techniques known to those of skill in the art.

The compounds tested as modulators of PEDF-R protein can be any small chemical compound, or a biological entity, such as a protein, e.g., an antibody, a sugar, a nucleic acid, e.g., an antisense oligonucleotide, siRNA, or a ribozyme, or a lipid. Alternatively, modulators can be genetically altered versions of a PEDF-R protein. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries (see, e.g., U.S. Pat. No. 5,010,175, Furka, Int. J Pept. Prot. Res., 37: 487-493, 1991; Houghton, et al., Nature, 354: 84-88, 1991). Other chemistries for generating chemical diversity libraries can also be used. Such chemistries include, but are not limited to: peptoids (e.g., PCT Publication No. WO 91/19735), encoded peptides (e.g. PCT Publication No. WO 93/20242), random bio-oligomers (e.g. PCT Publication No. WO 92/00091), benzodiazepines (e.g., U.S. Pat. No. 5,288,514), diversomers such as hydantoins, benzodiazepines and dipeptides (Hobbs, et al., Proc. Nat. Acad. Sci. U.S.A., 90: 6909-6913, 1993), vinylogous polypeptides (Hagihara, et al., J. Amer. Chem. Soc., 114: 6568, 1992), nonpeptidal peptidomimetics with glucose scaffolding (Hirschmann, et al, J. Amer. Chem. Soc., 114: 9217-9218, 1992), analogous organic syntheses of small compound libraries (Chen, et al., J. Amer. Chem. Soc., 116: 2661, 1994), oligocarbamates (Cho, et al., Science, 261: 1303, 1993), and/or peptidyl phosphonates (Campbell, et al., J. Org. Chem., 59: 658, 1994), nucleic acid libraries (see Ausubel, Berger and Sambrook, all supra), peptide nucleic acid libraries (see, e.g., U.S. Pat. No. 5,539,083), antibody libraries (see, e.g. Vaughn, et al., Nature Biotechnology, 14: 309-314, 1996 and PCT/US96/10287), carbohydrate libraries (see, e.g., Liang, et al., Science, 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like).

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In one embodiment, the invention provides solid phase based in vitro assays in a high throughput format, where the cell or tissue expressing the PEDF-R protein is attached to a solid phase substrate. In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100-about 1500 different compounds. It is possible to assay many plates per day; assay screens for up to about 6,000, 20,000, 50,000, or 100,000 or more different compounds are possible using the integrated systems of the invention.

In one embodiment the invention provides soluble assays using a PEDF-R protein or PEDF-R ligand, or a cell or tissue expressing a PEDF-R protein or PEDF-R ligand protein, either naturally occurring or recombinant. In another embodiment, the invention provides solid phase based in vitro assays in a high throughput format, where the PEDF-R protein is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

The protein of interest, or a cell or membrane comprising the protein of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example; where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc., 85: 2149-2154, 1963 (describing solid phase synthesis of, e.g., peptides); Geysen, et al., J Immun. Meth., 102: 259-274, 1987 (describing synthesis of solid phase components on pins); Frank, et al., Tetrahedron, 44: 60316040, 1988 (describing synthesis of various peptide sequences on cellulose disks); Fodor, et al., Science, 251: 767-777, 1991; Sheldon, et al., Clinical Chemistry, 39: 718-719, 1993; Kozal, et al., Nature Medicine, 2: 753759, 1996 (all describing arrays of biopolymers fixed to solid substrates). Non-chemical approaches for fixing tag binders to substrates include other common methods, such as heat, cross-linking by UV radiation, and the like.

Nucleic acid assays for detecting the presence of DNA and RNA for a PEDF-R polynucleotide in a sample include numerous techniques known to those skilled in the art, such as Southern analysis, Northern analysis, dot blots, RNase protection, S1 analysis, amplification techniques such as PCR and LCR, and in situ hybridization. In situ hybridization, for example, the target nucleic acid is liberated from its cellular surroundings so as to be available for hybridization within the cell while preserving the cellular morphology for subsequent interpretation and analysis. The following articles provide an overview of the art of in situ hybridization: Singer, et al., Biotechniques, 4: 230-250, 1986; Haase, et al., METHODS IN VIROLOGY, Vol. VII, pp. 189-226, 1984; NUCLEIC ACID HYBRIDIZATION: A PRACTICAL (Hames, et al., eds., 1987). In addition, a PEDF-R protein can be detected using the various immunoassay techniques described above. The test sample is typically compared to both a positive control (e.g., a sample expressing a recombinant PEDF-R protein) and a negative control.

The present invention also provides for kits for screening for modulators of PEDF-R proteins or nucleic acids. Such kits can be prepared from readily available materials and reagents. For example, such kits can comprise any one or more of the following materials: PEDF-R nucleic acids or proteins, reaction tubes, and instructions for testing PEDF-R activity. Optionally, the kit contains a biologically active PEDF-R protein.

For use in diagnostic, research, and therapeutic applications suggested above, kits are also provided by the invention. In the diagnostic and research applications such kits can include any or all of the following: assay reagents, buffers, PEDF-R specific nucleic acids or antibodies, hybridization probes and/or primers, antisense polynucleotides, ribozymes, dominant negative PEDF-R polypeptides or polynucleotides, small molecules inhibitors or activators of PEDF-R, and the like. A therapeutic product can include sterile saline or another pharmaceutically acceptable emulsion and suspension base as described above.

Accordingly, kits of the present invention can contain any reagent that specifically hybridize to PEDF-R nucleic acids, e.g., PEDF-R probes and primers, and PEDF-R-specific reagents that specifically bind to and/or modulate the activity of a PEDF-R protein, e.g., PEDF-R antibodies, PEDF-R ligands, or other compounds, are used to treat PEDF-R-associated diseases or conditions. Kits of the present invention can also contain additional agents that can be administered concomitantly with the compounds of the present invention.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g.; CD ROM), and the like. Such media can include addresses to internet sites that provide such instructional materials.

The following Examples of specific embodiments for carrying out the present invention are offered for illustrative purposes only, and are not intended to limit the scope of the present invention in any way.

The disclosures of all publications, patents and patent applications cited herein are hereby incorporated by reference in their entirety.

### EXAMPLES

PEDF acts in neuronal survival and differentiation in the retina and CNS. It also acts, inter alia, in excluding vessels from invading the retina, vitreous, and aqueous, as well as vessels from nourishing tumors. PEDF is a secreted protein and a member of the serpin family of serine protease inhibitors, but its neurotrophic and antiangiogenic effects are independent from serine protease inhibition. Molecules that interact with PEDF were identified to elucidate the mechanisms of action that mediate the biological activities of PEDF. Using a yeast 2-hybrid system, 27 clones with a potential for interacting with PEDF were identified. One of them is a cDNA isolated from human eye, normal pigmented epithelium (Accession BC017280, GI: 16878146), termed R1. It codes for a polypeptide of 504 amino acids (MW 55,315 Da) with four potential N-glycosylation sites and four transmembrane domains. R1 has similarity to an unknown human liver orphan (Accession X56789, GI: 34763) and human transport-secretion protein 2 (TTS-2, GI: 9663151) and it mouse homologue (GI: 12833095). They share sequence homology with adiponutrin (Accession NP_{—}473-429.1, GI: 16905119), a transmembrane protein corresponding to a novel dietary- and obesity-linked mRNA specifically expressed in the adipose lineage (see alignment of sequences). See also Baulande et al., JBC, 276: 33336-33344, 2001. The fragment of cDNA from R1 with the PEDF-binding region was overexpressed using Gateway Technology as a fused His tagged polypeptide and termed p12. Purification of p12 yielded a protein with binding affinity to purified PEDF. Native human RPE, retina and several RPE cell lines expressed p12, as demonstrated by PCR. Thus a gene that codes for a PEDF receptor was identified. The derived polypeptide has regions of homology to transmembrane domains and a presumptive extracellular region that has binding affinity for PEDF. Baulande, et al., JBC, 276: 33336-33344, 2001.

The plasmid for R1 from ATCC was used to generate p12. p12 protein was generated by expressing a cDNA obtained by PCR with oligonucleotide primers designed from the nucleotide sequence of clone p12 provided by Julio Escribano and Jorge Laborda and following Gateway Technology (Invitrogen).

In addition, the R1 sequence contains a patatin-like phospholipase region (protein family data base accession number PF01734; see http://www.sanger.ac.uk/Software/Pfam). The patatin family consists of various soluble glycoproteins from potato tubers believed to function as storage proteins. They have the enzymatic activity of lipid acyl hydrolase, catalyzing the cleavage of fatty acids from membrane lipids, i.e., phospholipase A (PLA1, and PLA2) activities that hydrolyze both acyl groups from the glycerol backbone of phospholipids to produce free fatty acids and glycerol phosphate. Members of this family have been found also in vertebrates.

Under physiologic conditions, PLA2 can be involved in phospholipid turnover, membrane remodeling, exocytosis, detoxification of phospholipid peroxides, and neurotransmitter release. However, under pathological situations, increased PLA2 activity may result in the loss of essential membrane glycerophospholipids, resulting in altered membrane permeability, ion homeostasis, increased free fatty acid release, and the accumulation of lipid peroxides. PLA2 is a key enzyme involved in the release of arachidonic acid (AA) from the cell membrane. Inhibition of PLA2 by lipocortins also results to a decrease in inflammation.

PCR amplification confirmed p12 expression in human RPE, RPE cells lines and retina. The R1 and p12 cDNAs were overexpressed in E. coli as fused His tagged polypeptides and the recombinant proteins purified by NTA affinity column chromatography. Binding assays by His-tag pull-down, solid-phase binding, ultrafiltration and SPR showed that both R1 and p12 bound specifically to PEDF and that these interactions were insensitive to 500 mM NaCl.

PLA activity in R1 was evaluated by a continuous spectrophotometric assay based on a coupled enzymatic reaction with lipoxygenase and [1,2-dilinoleoly]phosphatidylcholine as the PLA substrate. The linoleic acid released by PLA is oxidized by the coupling enzyme and the PLA activity is measured by the increase of absorbance of the product, hydroperoxide of linoleic acid, at 234 nm. Optimal conditions for R1 solubility and activity were investigated. R1 exhibited PLA activity that varied with buffer and pH conditions with a maximum PLA solubility and activity obtained with 10 mM Tris-C1, pH 7.5 and 3 mM deoxycholate-Na The PLA activity was specific since no activity was detected in the absence of R1.

As discussed above, PEDF-R1 has PLA activity and probably a lipase activity. Non confluent and growing NIH3T3-L1 cells and an RPE cell line (ARPE19) can be induced for adipogenesis differentiation in the presence or absence of exogenous human recombinant PEDF protein. RNA expression for PEDF and for PEDF-R1 can be analyzed, by qRT-PCR (qRT-PCR is described in detail below) using cDNA samples prepared from RNA of undifferentiated and differentiated cells, either treated or untreated with PEDF. Differentiated NIH3T3-L1 adipocytes and ARPE19 cells would be expected to show a decreased level of PEDF expression relative to the undifferentiated preadipocyte cells. In contrast, expression of PEDF-R1 would be expected to increase with adipogenesis being higher in adipose cells than in preadipocytes and in ARPE19 cells uninduced for adipogenesis. PEDF treated cells would be expected to show a decrease in PEDF-R1 as reflection of maintenance of an undifferentiated (non adipogenetic) status. At the molecular level, upon interaction with PEDF-R1, PEDF may activate its PLA and lipase activities, which catalyze the release of fatty acids from phospholipids and triglycerides from membranes and lipids droplets. These activities may result in a reduction of lipid droplets within the adipocyte cells and thus reducing its adipocyte state.

Thus, a novel gene R1 has been identified and disclosed herein. R1 is expressed in the RPE and retina that codes for a putative PEDF receptor protein with extracellular ligand-binding, transmembrane and intracellular domains and a phospholipase A active region. These observations suggest that upon binding to PEDF, the R1 receptor may trigger a change in free fatty acid release. Free fatty acid release is a novel metabolic signal involved in neuronal survival and apoptosis. Thus a signal transduction pathway initiated by PEDF-R1 interactions can be involved in neurotrophic and antiangiogenic activities of PEDF.

There is proteomic evidence that the levels of pigment epithelium-derived factor (PEDF) protein, a soluble molecule with potent antiangiogenic and neurotrophic properties, are differentially expressed in preadipocytes and mature adipocytes. Kratchmarova et al., Mol Cell Proteomics 1, 213-22, 2002. PEDF is highly secreted by preadipocytes but not adipocytes. The role PEDF has on adipogenesis was investigated using a Chemicon® assay in which differentiation of NIH3T3-L1 preadipocytes to mature adipocytes is induced with dexamethasone, isobutylmethylxanthine (IBMX) and insulin to accumulate intracellular lipids. Staining the cells with Oil Red O can reveal the intracellular lipid droplet accumulation under the microscope. Quantification can be accomplished by measuring the extracted lipid stain spectrophotometrically. Exogenous additions of PEDF at 5 and 50 nM to the cultures decreased the Oil Red O staining in the cells and the absorbance of the extracts similar to the effects of the cytokine transforming growth factor beta (TGFβ). These results demonstrate that PEDF can interfere with lipid accumulation in cells undergoing adipocyte maturation and that PEDF plays a role in adipogenesis.

### Yeast-2-Hybrid System

A two-hybrid system of Saccharomyces cerevisiae (Clontech, Palo Alto, Calif., USA) was used. The human cDNA PEDF was cloned into pAS2-1 (Clonetech) next to the 5′ end of the Binding Domain (BD). The resulting vector was used a bait. The target was a human liver cDNA library (Human Liver MATCHMAKER cDNA Library de Clontech) with about 3×10⁶ independent clones of 2 kb (average size) in a pACT2 vector. The 5′ end of all the clones were next to the Activation Domain (AD) of the Gal4 transcription factor.

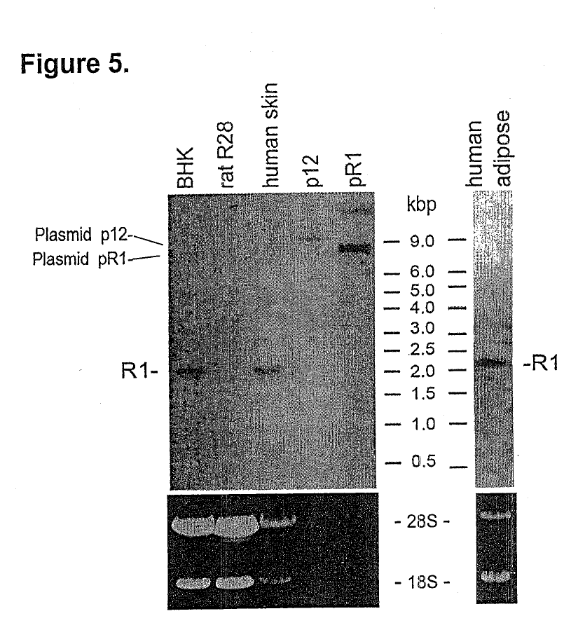

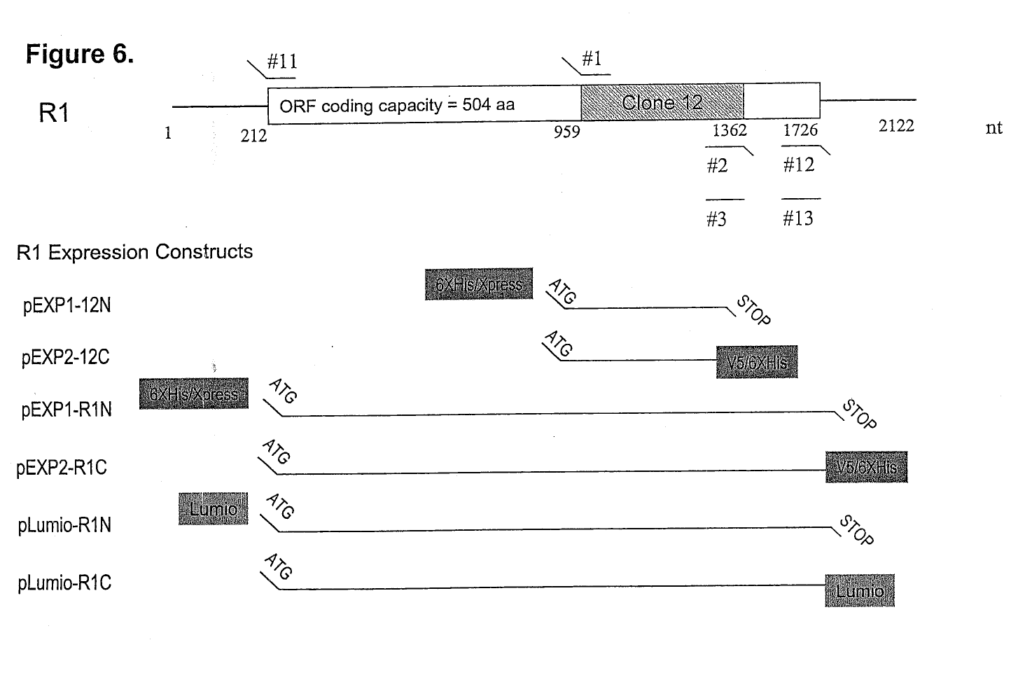

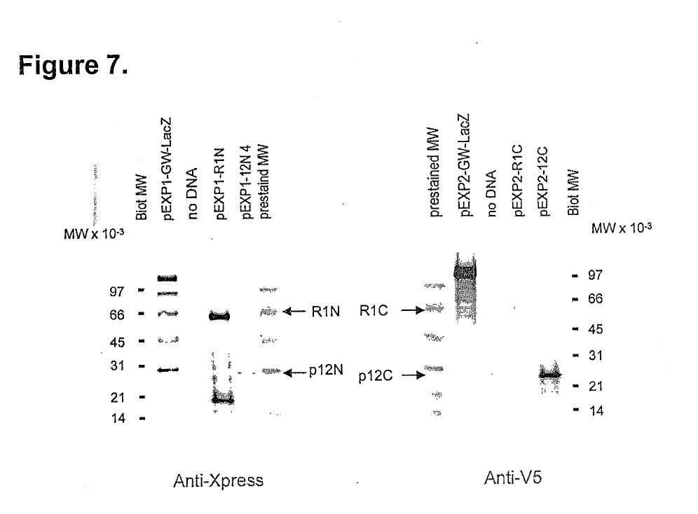

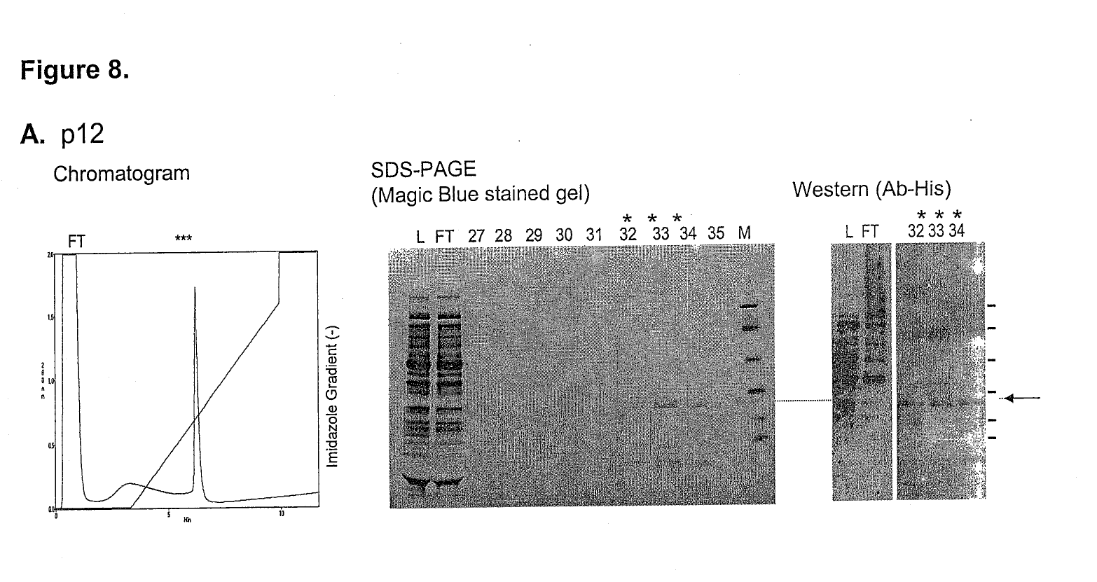

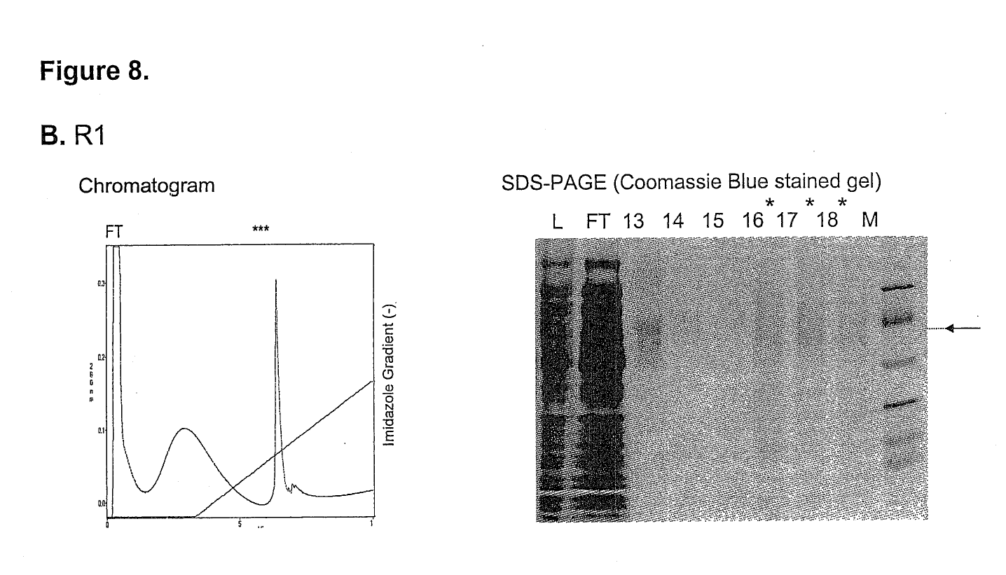

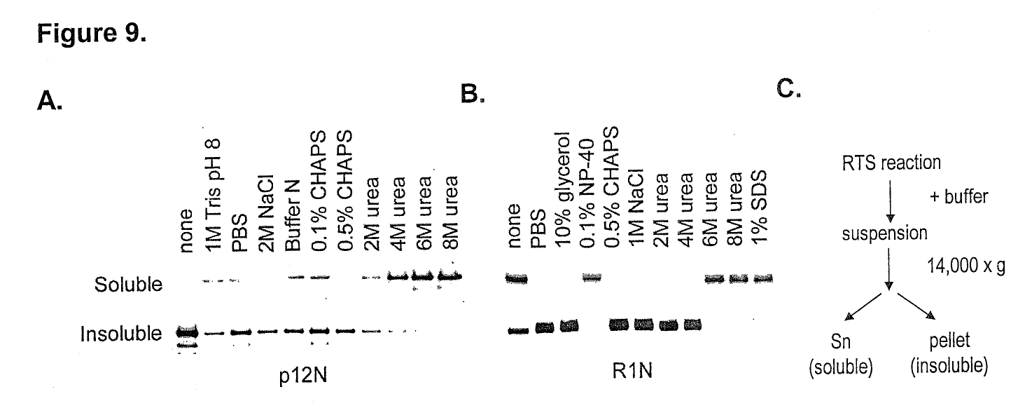

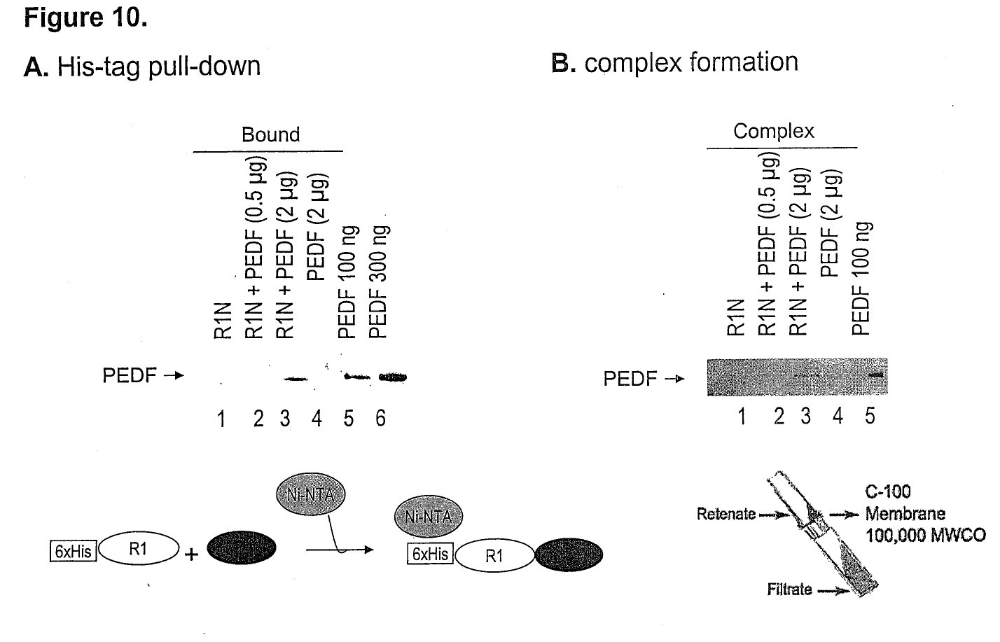

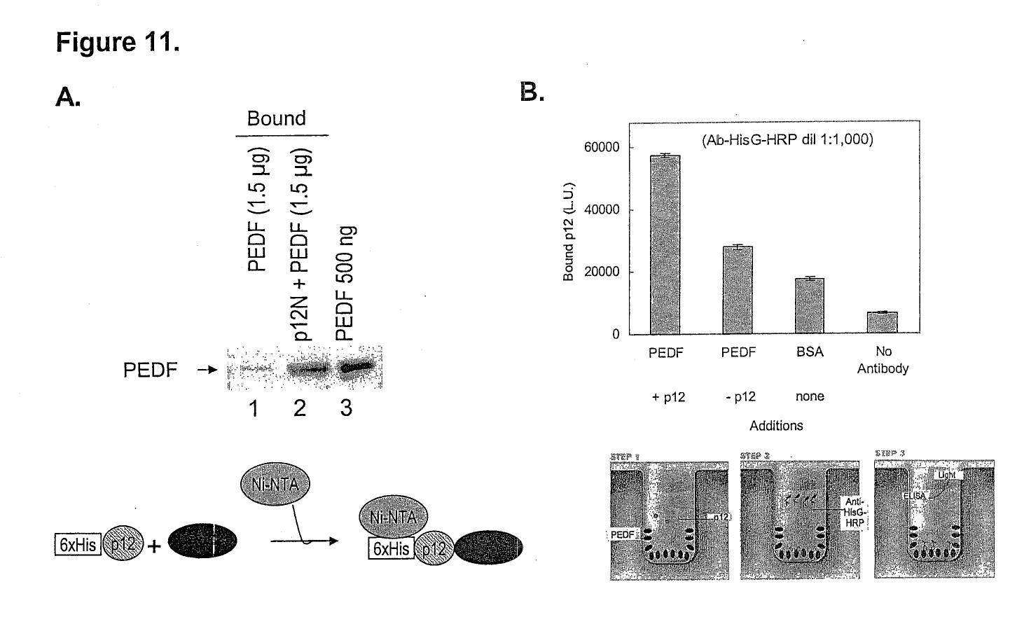

To localize the receptor binding site on PEDF, Saccharomyces cerevisiae CG-1945 were transformed with C-terminal deletions of PEDF in pAS2-1 (see FIG. 5 poster) and the liver cDNA library. The interaction between BD and AD was analyzed by the 3-Amino Triazol (3-AT) assay, performed in media without histidine but with increasing concentrations of 3-AT (0-100 mM).

DNA sequencing was performed using ABI PRISM 310 (Applied Biosystems, CA). A particular clone (clone 12) was identified to contain the potential PEDF interacting fragment.

### Sequence Analyses

Nucleotide sequences were analyzed and compared with the GenBank Databases using Blast Search (http://www.ncbi.nlm.nih.gov/BLAST/). Alternatively SIB BLAST Network Service (http://us.expasy.org/tools/blast/) was used, which led to the discovery of R1. Protein sequences were aligned and compared using SIM-Alignment Tool for protein sequences (http://us.expasy.org/tools/sim-prot.html). For multiple alignment among selected sequences T-Coffee program (T-Coffee: A novel method for multiple sequence alignments” Notredame, et al., Journal of Molecular Biology, 302: 205-217, 2000; http://us.expasy.org/cgi-bin/hub) was used. TMpred was used for prediction of polypeptide transmembrane regions and orientation (Hofmann, et al., Biol. Chem. Hoppe-Seyler, 374:166, 1993; http://www.ch.embnet.org/software/TMPRED_form.html). Potential sites for N-glycosylation were predicted using NetNGlyc 1.0 Server (http://www.cbs.dtu.dk/services/NetNGlyc/).

### PCR

Primers for screening the expression of p12 sequence were 12-forward, 5′ AAC CCC TTG CTG GCG TTG C 3′; and 12-reverse, 5 CCC GTC TGC TCC TTC ATC C 3′. Templates were R1 cDNA, cDNAs prepared from human retina, human RPE, ARPE-19 and human TERT in PCR SuperMix reactions following instructions by manufacturer (Invitrogen).

### Construction of P12 Clones

Oligonucleotide primers were designed to flank the DNA fragment containing the PEDF interacting region obtained form the yeast-2 hybrid. The forward primer #1 was 5′ Cacc aTG CAG CGG AAC GGC CTC CTG AAC C 3′ (Cacc+gene specific). Two reverse primers were: #2, 5′ Cta GTT CCT CTT GGC GCG CAT CAC C 3′ (gene specific+stop) and #3, 5′ GTT CCT CTT GGC GCG CAT CAC C 3′ (gene specific). PCR reactions with primers #1 and 2 were set with R1 as template to amplify p12 with a ATG and Stop codon. PCR reactions with primers #1 and 3 were set with R1 as template to amplify p12 with only the ATG codon. The PCR products were inserted into entry vectors pENTR-TOPO-D and pENTR-TOPO-SD, respectively by the TOPO reactions (Invitrogen). The p12 inserts were recombined into expression vectors pEXP-DEST-1 and pEXP-DEST-2, respectively, using LR recombinase (Invitrogen). The resulting plasmids were termed pEXP-12N and pEXP-12C and contained p12 sequences with a fusion His Tag at the N-terminus and C-terminus, respectively. The derived recombinant polypeptide from the pEXP-12N was termed p12N, and the one from pEXP-12C was termed p12C.

### Expression of P12

The p12 DNA sequence was engineered to have six histidines on either the N-terminus or the C-terminus, and the chimeric gene was cloned into an expression vector using Gateway Technology cloning and TOPO vectors (Invitrogen). Expression of the peptide was performed using In Vitro Protein Synthesis system (IVPS) by Invitrogen and this expression vector. Reactions were conducted using E. coli extract, T7 RNA polymerase, and the DNA expression template. The resulting mixture was analyzed by western blot and purified using a Nickel-Nitrilotriacetic acid (Ni-NTA) column. The resulting p12 constructs fused to a 6×His at its N-terminus were termed p12N and at its C-terminus p12C.

### Batch Purification

Purification of the p12N peptide was accomplished using the ProBond Purification system (Invitrogen) with Ni-NTA resin in a batch method under denaturing and native conditions. Protein in the particulate fraction of the IVPS was purified using chaotropic agents, as follows. The insoluble portion of the IVPS reaction was resuspended in Guanidinium Lysis Buffer (6M Guanidine HCl, 20 mM Na₃PO₄, pH 7.8, 500 mM NaCl), and 50 μl of this solution were added to 50 μl of Ni-NTA resin. The mixture was rotated for 1 hour at 25° C., centrifuged at 3000 rpm 15 min, and the supernatant removed. Subsequent washes were performed twice, each with 200 μl of 8M Urea, 20 mM Na₃PO₄, 500 mM NaCl at pH 7.8 and repeated with the same buffer but at pH 6.0 and then pH 5.3. Then proteins were eluted from the beads with sample buffer.

### Automated Purification

To purify the p12N peptide at larger scale, a Poros MC column attached to a BioCad 700E computerized system was used. The column was activated with NiCl₂. Polypeptide p12N was purified from the soluble material from the expression mixture diluted in binding buffer (50 mM Na-phosphate, 0.5 M NaCl pH 8.0). 2.5 ml of the sample were injected into the column followed by extensive washes with binding buffer. Polypeptide p12N was eluted with a linear gradient of 0-80 mM imidazole and the eluate was collected into separate fractions.

### Western Blotting

Samples containing p12 were resolved by SDS-PAGE and transferred to nitrocellulose membrane. The membrane was blocked in 5% BSA in TBS-T, and incubated for 1 hour at room temperature with a 1:10,000 dilution of Anti-HisG-HRP (Invitrogen). This was followed by three washes for 5 minutes with TBS-T, incubation in Lumi-Light substrate solution (Roche Molecular Biochemicals) and exposure to Biomax ML film (Kodak). Alternatively, not labeled mouse Anti-His antibody (Amersham) was used in a 1:30,000 dilution (for Lumi-Light detection) or a 1:3,000 dilution (for colorimetric detection), followed by incubation in HRP-labeled Goat Anti-Mouse IgG(H+ L). The membrane was then incubated as previously described for the Lumi-Light method or in ABC solution (Vectastin ABC elite kit) for calorimetric method. For samples containing PEDF, a polyclonal antibody to PEDF was used in a 1:2,000 dilution followed by incubation with biotinylated anti-rabbit antibody (1:1000 dilution) and incubation in ABC solution. Color was developed with HRP color development reagent.

### Binding Assays

PEDF was immobilized on 96-well plates. Blocking solution (1% BSA in PBST) was added to block non specific sties for 1 hour at room temperature. Purified p12N polypeptide in solution was added to the wells and incubated at 4° C. for 16 hours. After washing with blocking solution, Anti-HisG-HRP antibody diluted at 1:1000 or 1:10,000 was added to the wells and incubated for 1 hour at room temperature. After washed with blocking solution, SuperSignal ELISA Femto (Pierce) substrates were added and incubated 2 minutes at room temperature with shaking. The amount of luminescence was measured using a luminometer (Wallac, Model 1450 Microbeta TRILUX). Negative controls included BSA immobilized on the plates instead of PEDF, no p12N, or no antibody added to the reactions.

Alternatively, p12N (estimated 0.8 μg) was immobilized on Ni-NTA resin beads in binding buffer (50 mM Na₃PO₄, 0.5M NaCl, pH 8.0) by incubation with rotation at room temperature for 30 min. The beads were washed with binding buffer three times. Recombinant human PEDF protein (3 μg) in binding buffer was added to the beads and incubated by rotation for three hours at room temperature. The beads were washed three times with binding buffer. Sample buffer was added to the beads mixed and heated at 100° C. for 3-5 minutes. The extracted proteins were subjected to SDS-PAGE and western transfer. The proteins were detected in the membrane by Ponceau Red and immunostaining with antiserum to PEDF, Ab-rPEDF;

Overexpression of R1cDNA fragments.

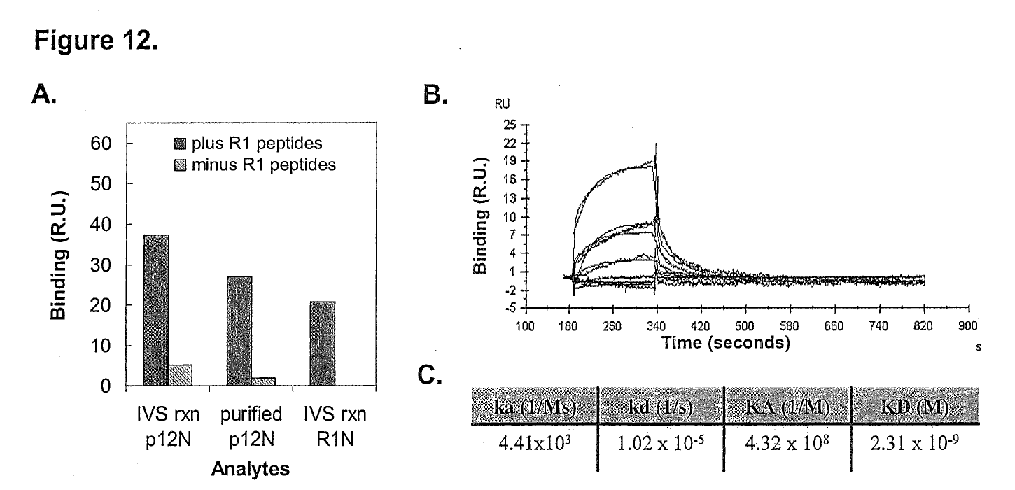

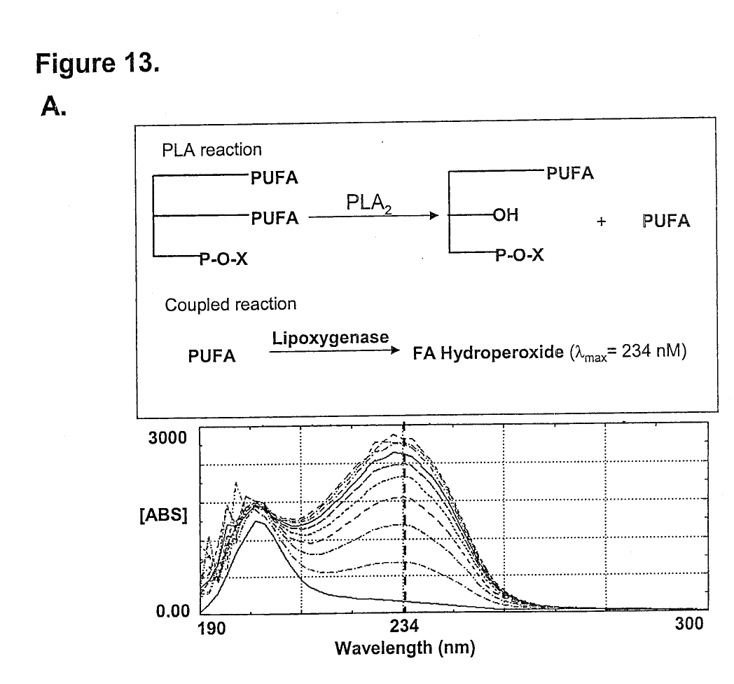

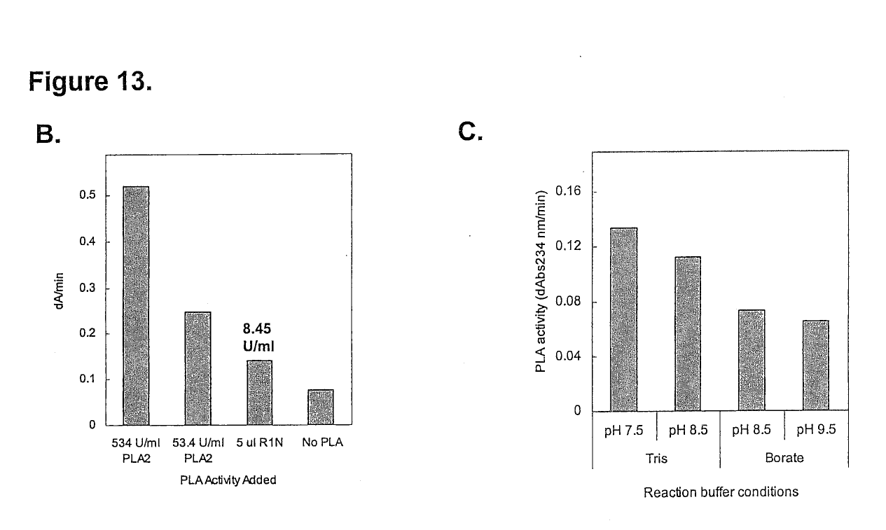

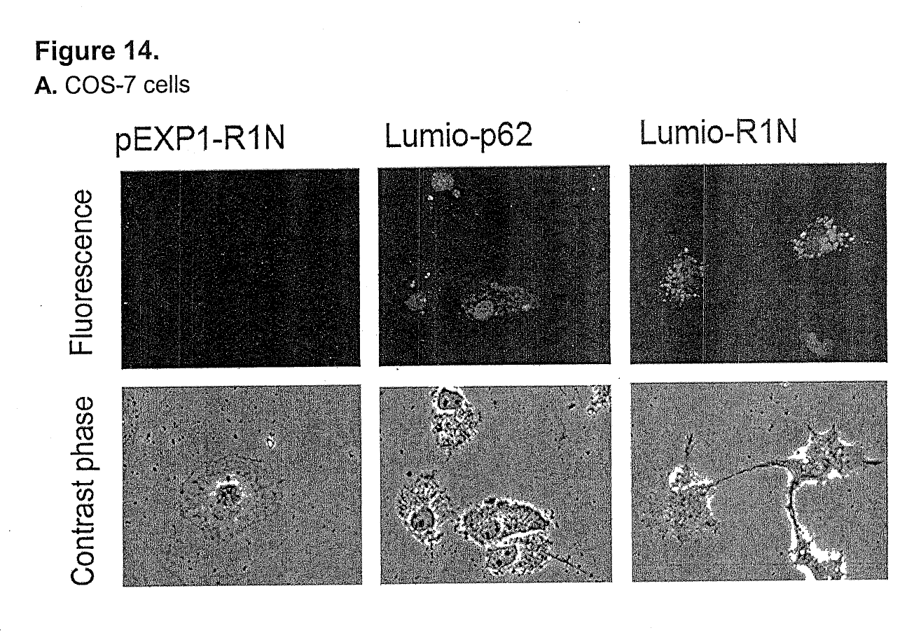

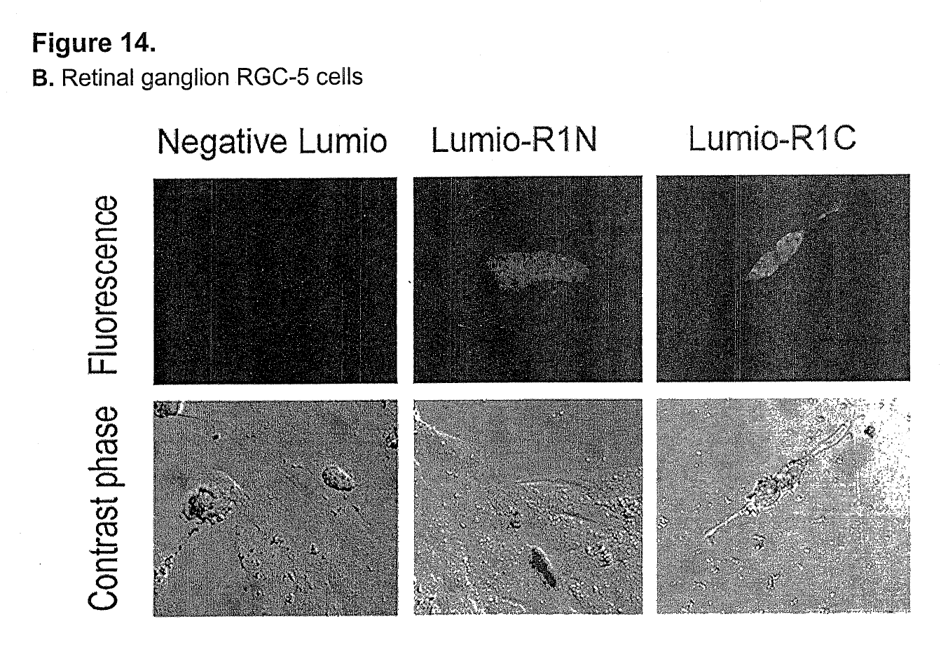

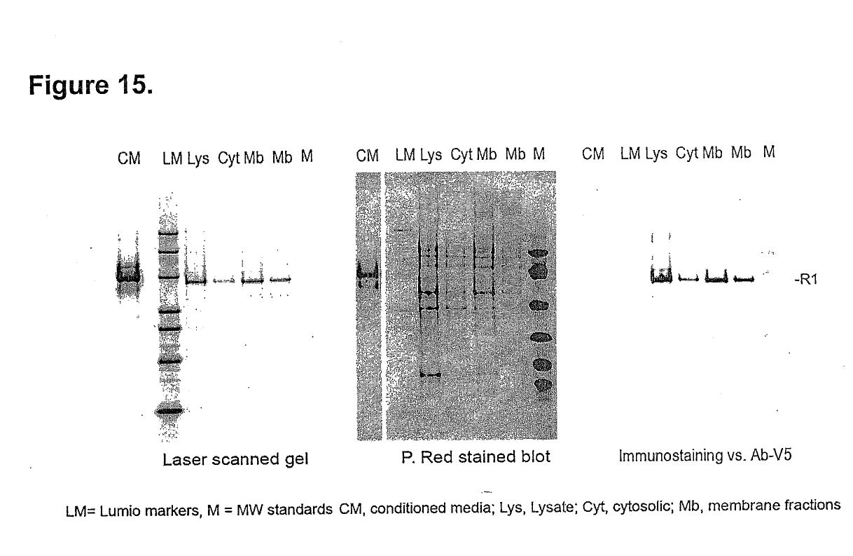

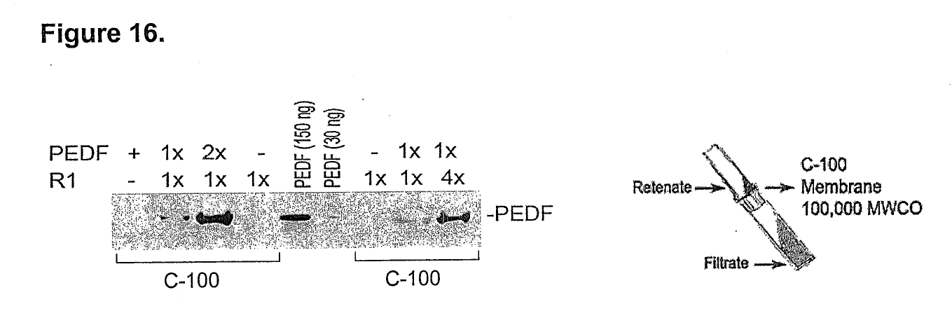

Fragments of R1 cDNA were recombined from pENTR-vectors containing R1N or R1C with or without initiation and termination codons (see scheme in FIG. 12) into pEXP 1-Dest, pEXP2-Dest, pcDNA6.2/nLumio-Dest or pcDNA6.2/cLumio-Dest (Invitrogen) using LR clonase (Invitrogen). The final constructs are summarized in FIG. 12. Constructs were propagated in DH5α E. coli cells. The pEXP vectors contain 6×His tags, and in addition the pEXP1 vectors contain Xpress epitopes and the pEXP2 ones have V5 epitopes. The pEXP vectors contained the R1 fragments under the control of bacterial T7 transcriptional promoter. Both pLumio vectors contained Lumio tags and V5 epitopes, and the R1 fragments under the mammalian CMV transcriptional promoter.

Expression of polypeptides was performed using an in vitro protein synthesis system from E. coli extracts (Expressway™ Plus Expression System, Invitrogen) from the different expression vectors. Alternatively, the RTS-100 or RTS-500 (Roche) was used. Reactions were conducted using T7 RNA polymerase and the DNA expression as template. The resulting mixture was analyzed by Western Blotting and the R1 polypeptides purified using a Nickel-Nitrilotriacetic acid column.

### Automated Purification.

To purify the R1 and p12 polypeptides, a Poros MC column attached to a BioCad 700E computerized system was used. The column was activated with NiCl₂. R1 or p12 were purified from the soluble material from the expression reaction mixture diluted in binding buffer (50 mM Na-phosphate, 0.5 M NaCl pH 8.0). Mixtures of not more than 5 ml were injected into the column pre-equilibrated with binding buffer. Unbound material (FT) was washed several times with binding buffer. Bound R1 polypeptides were eluted with a 0-80 mM imidazole linear gradient and collected into separate fractions. The fractions were resolved by SDS-PAGE in a 10-20% polyacrylamide gradient gel and stained with Magic Blue staining solution.

### Western Blotting.

Samples containing R1 polypeptides were resolved by SDS-PAGE and transferred to nitrocellulose membrane. The membrane was incubated with a 1:10,000 dilution of Anti-HisG-HRP (Invitrogen). This was followed by incubation in Lumi-Light substrate solution (Roche Molecular Biochemicals) and exposure to film. Alternatively, anti-His antibody (Amersham) were used in a 1:30,000 dilution (for Lumi-Light detection) or a 1:3,000 dilution (for colorimetric detection), followed by incubation in HRP-labelled Goat Anti-Mouse IgG(H+L) and incubation in Lumi-Light substrate solution for the Lumi-Light method or incubation in ABC solution (Vectastin ABC elite kit) for calorimetric method. Anti V5 (1:5000) and anti-Xpress (1:5000) (Invitrogen) were used for colorimetric detection as above. For samples containing PEDF, a rabbit polyclonal antibody to PEDF was used in a 1:2,000 dilution followed by incubation with biotinylated anti-rabbit and incubation in ABC solution. Color was developed with HRP color development reagent.

### Solubility Studies on Recombinant R1 and P12 Polypeptides.

Aliquots of reaction mixtures containing R1 polypeptides were diluted in different buffer conditions with additives. After incubation at 25° C. for 3 minutes, the soluble (Sn) and particulate (pp) material was fractionated by centrifugation (14,000×g, 25° C., 15 min). The Sn ws precipitated with acetone to remove components of the reaction mixtures that interfere with the migration by the SDS-PAGE. Alternatively, RTS-100 reaction mixture (100 μl) was diluted 1:10 with PBS, and the particulate fraction after centrifugation (15 minutes at 14,000×g, 4° C.), resuspended in buffers containing different additives. R1 polypeptides in Sn and pp fraction were detected by western blotting.

### His-Tag Pull-Down Assay.

Binding to PEDF to R1 polypeptides was assayed by precipitating a complex formed between PEDF and the R1 polypeptides containing 6×His tag fused peptides with NiNTA resin beads (Invitrogen). For R1-PEDF binding, soluble fractions of RTS-500 reaction mixtures containing R1N polypeptides (estimated about 700 ng R1N) were mixed with PEDF protein (1 or 4 μg), in binding buffer (50 mM phosphate-Na pH 7.5, 500 mM NaCl, 1% NP-40, 104 μl final volume). Binding reaction mixtures were incubated at 4° C. with gentle rotation for 2 hours, and then NiNTA resin beads pre-equilibrated in binding buffer were added, and the suspension incubated for 1 hour at 4° C. with gentle rotation.

For p12-PEDF binding, Ni-NTA resin beads (50 μl) were mixed with 20 μl purified p12N (fraction 33 of BioCad purification) diluted in 180 μl binding buffer (50 mM phosphate-Na pH 8.0, 0.5 M NaCl,) (estimated to be about 800 ng of p12N). They were incubated and rotated for 30 min. and washed three times with binding buffer. PEDF (3 μg) in binding buffer was added to the beads suspensions and rotated 3 hrs.

The beads were sedimented by centrifugation, washed three times with binding buffer and the proteins extracted with SDS-PAGE sample buffer (50 μl) analyzed by Western Blotting against PEDF.

### Complex Formation Assay.

This assay is based on the separation of complexes formed between PEDF and R1 by size-ultrafiltration. R1-PEDF complexes greater than 100-kDa, are retained by a membrane of Mr 100,000 exclusion limit, while free PEDF molecules of 50 kDa are filtered through. For R1-PEDF binding, soluble fractions of RTS-500 reaction mixtures containing R1N polypeptides (estimated about 700 ng R1N) were mixed with PEDF protein (1 or 4 μg), in binding buffer (50 mM phosphate-Na pH 7.5, 500 mM NaCl, 1% NP-40, 104 μl final volume). Binding reaction mixtures were incubated at 4° C. with gentle rotation for 2 hours. Mixtures were diluted in binding buffer containing only 0.02% NP-40 and applied to Centricon-100 devices and centrifuged at 1000×g at 4° C. for 40 minutes or until most solution went through membrane, and the concentrated material was washed with the same buffer two more times. Half of the retained material was resolved by SDS-PAGE and visualized by immunostaining against anti-PEDF antibodies.

### Solid Phase Assay.

In this assay the binding between R1 polypeptides and PEDF are performed with immobilized PEDF on plastic. Detection of bound R1 is via luminescence from anti-HisG-HRP bound to the 6×His tag fused to the R1 polypeptides. Reactions between p12 and PEDF were conducted using plastic 96-well plates.

PEDF was diluted in 0.6 M Sodium Citrate, 0.1 M Sodium Carbonate, pH 9.0 (from Pierce BupH Citrate-Carbonate Buffer Pack) and 500 μg of PEDF or BSA (negative control) were placed in each well. Wells were blocked with 1% BSA/PBST. Soluble p12N polypeptides were diluted in phosphate buffer, pH 7.4, added to the wells, and allowed to bind PEDF at 4° C. for 16 hours. The wells were washed three times with Phosphate Buffered Saline, 0.05% Tween-20 (PBST) to remove unbound p12, then Anti-HisG-HRP was added in a dilution of 1:10,000 or 1:1000 in PBST and incubated for 1 hr, 25° C. The wells were washed three times with PBST, and ELISA Femto working solution (100 μl, Pierce) was added to each well, incubated for 1 min., and then the luminescence was recorded by a luminometer.

### PEDF Binding Analysis by Surface Plasmon Resonance (Biacore).

A surface plasmon resonance (SPR) response is detected when two or more molecules interact at the level of the surface of a sensor chip, provoking changes in the angle of minimum reflected light intensity on this surface, i.e., the refractive index of the medium. The interactions between PEDF and p12 or R1 were analyzed by SPR using a BIAcore 3000 instrument (BIAcore, Uppsala, Sweden) with immobilized PEDF, as described (Meyer et al., JBC, 277: 45400-7, 2002). PEDF (4 ng) was immobilized on a CM5 sensor chip, by NHS/EDC activation, followed by covalent amine coupling of the protein to the surface. A reference surface without protein was prepared by the same procedure. Both surfaces were then washed with 0.5 M NaCl and then re-equilibrated with binding buffer (HBS-N, BIAcore). Different dilutions of a p12 or R1 solution were injected both on surfaces, followed by a 0.5 M NaCl regeneration step. Binding was measured by SPR responses and expressed in resonance units. (RU). One RU represents a change of 0.0001° in the angle of the intensity minimum. The unspecific binding of p12 estimated from the reference surface was subtracted by the binding on the PEDF. Another negative control, a solution containing the product of a control plasmid (LacZ) was also injected on the same surfaces. The results were analyzed using BIAevaluation software (BIAcore, Uppsala, Sweden) and reported on sensograms.

### Kinetic Analysis for R1-PEDF Interaction by Surface Plasmon Resonance (Biacore).

R1N polypeptides used for kinetic analysis were obtained following a fractionation procedure. Briefly, synthesis reaction was performed using IVPS system (Expressway plus, Invitrogen). The reaction mixture (1 ml total volume) was centrifuged at 4° C. in Eppendorf centrifuce at 14,000 rpm for 20 minutes. The pellet was resuspended in 1.5 ml of PBS, pH 7.4 (Biofluids), incubated on ice for 1 hour and then centrifuged again. The pellet from the second centrifugation was resuspended in 500 ml of PBS, pH 7.4 containing 0.1% NP-40 (Calbiochem). The mixture was incubated again for 1 hour at 4° C. and centrifuged as described above. The interaction kinetic between the supernatant enriched in R1N polypeptide and PEDF was analyzed using a BIAcore 3000 instrument with PBS, 0.1% NP-40 as running buffer. Increasing concentrations of R1N (34-550 nM) were injected on the PEDF surface, and the binding of the two molecules was analyzed. Kinetic evaluation was performed using a BIAevaluation software (BIAcore) and the binding curves were fitted using a 1:1 Langmuir binding model with drifting baseline.

### Phospholipase (PLA) Activity Assay.

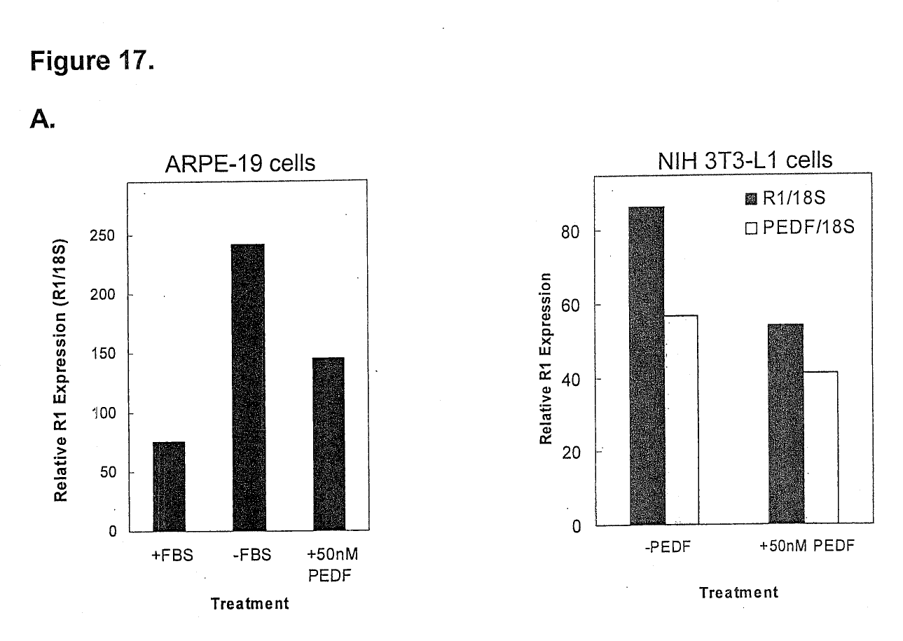

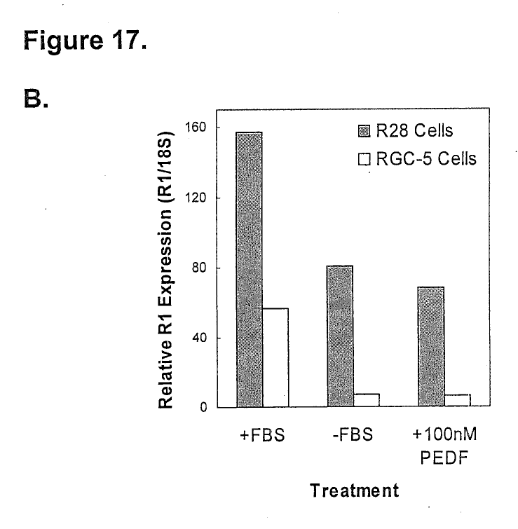

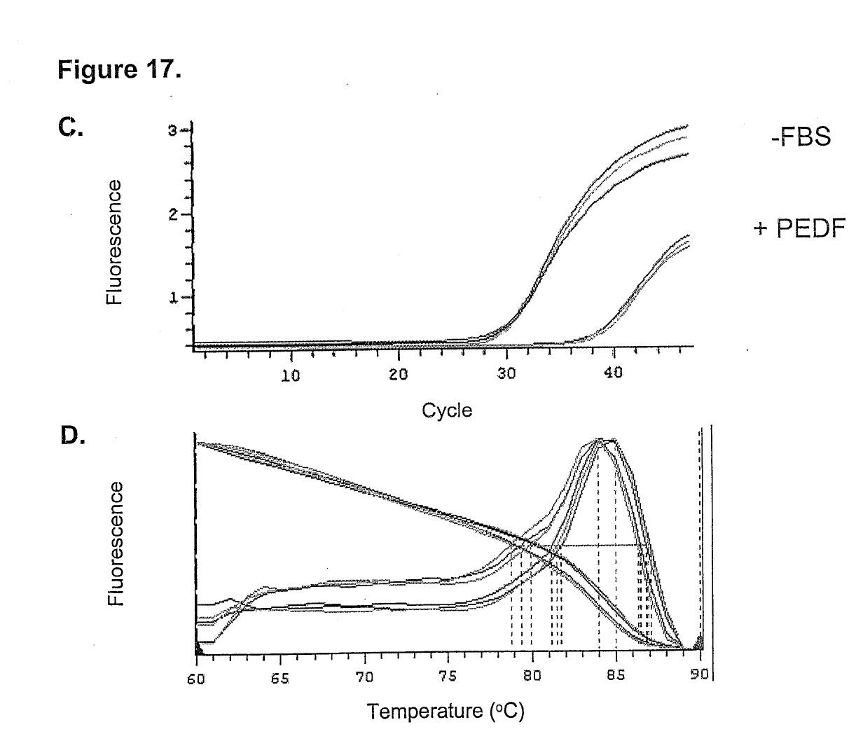

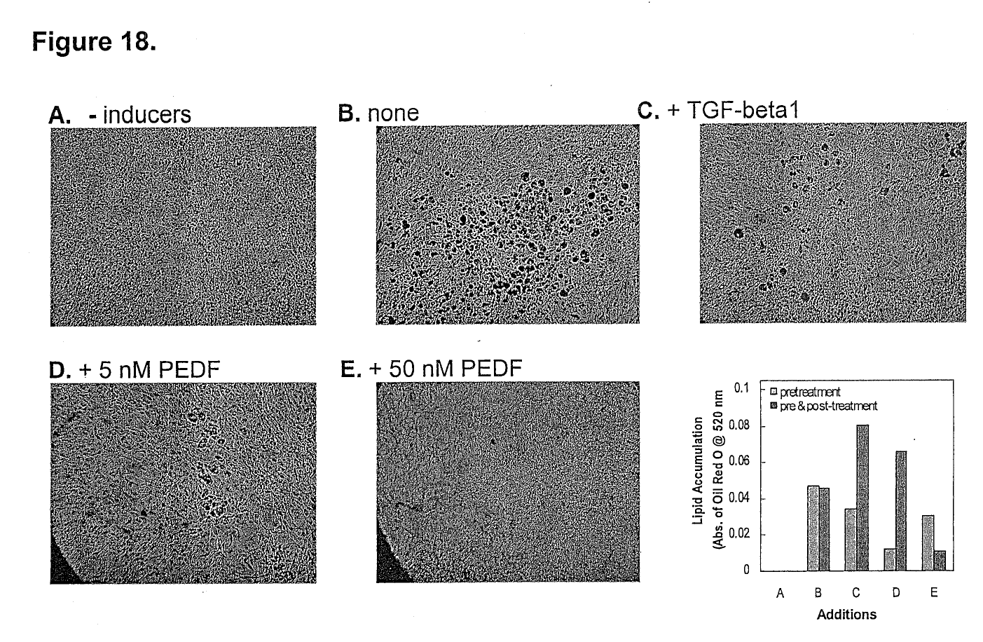

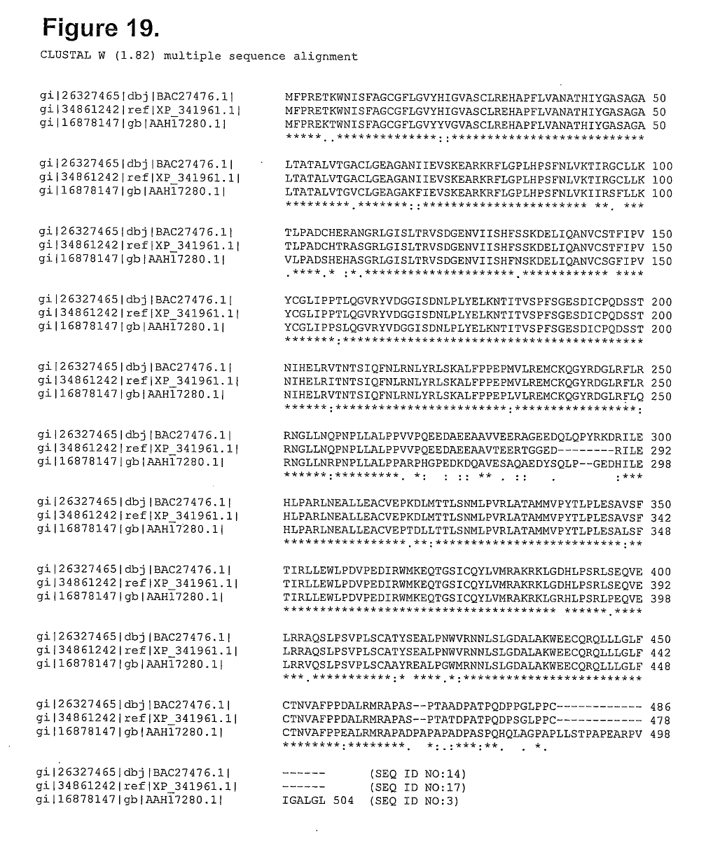

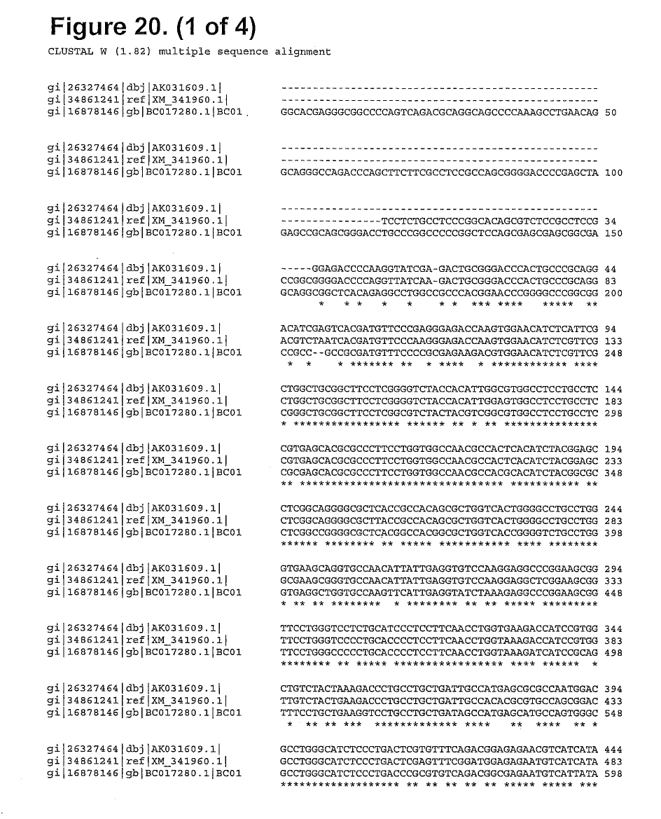

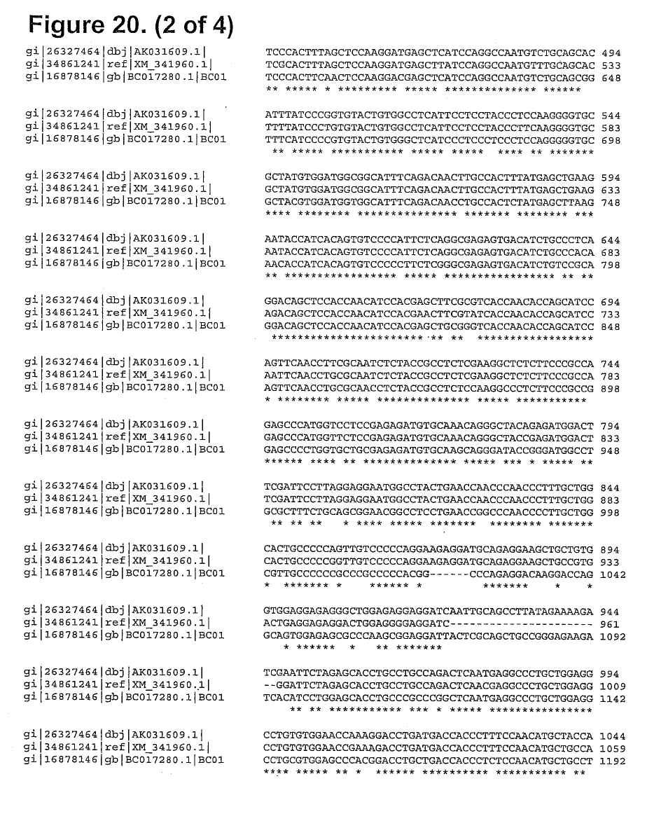

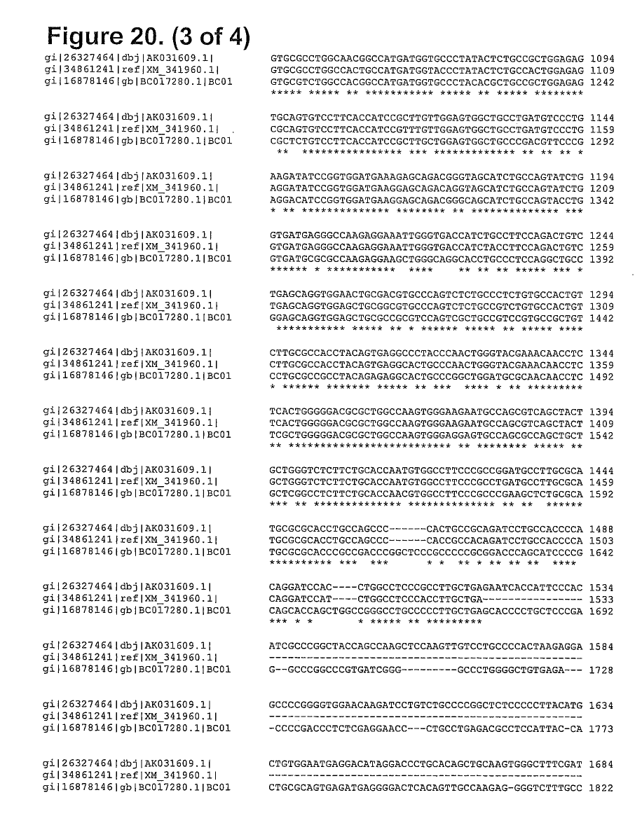

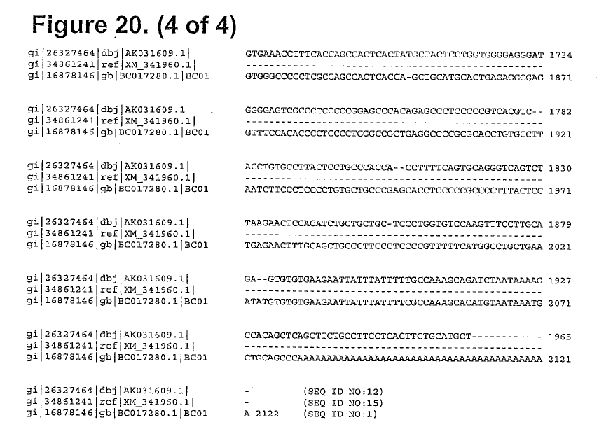

The PLA activity was spectrophotometrically determined as described previously (Jimenez-Atienzar, et al., Lipids., 38: 677-82, 2003) with minor modifications. As shown in Scheme (FIG. 17), this assay uses [1,2-dilinoleoyl]-phosphatidilcoline as phospholipase substrate and lipoxygenase as coupling enzyme. The phospholipase activity releases linoleic acid from the substrate, and the lipoxygenase oxidizes the released linoleic acid to form a derivative hydroperoxide. The PLA activity was followed spectrophotometrically by measuring the increase in absorbance at 234 nm as a result of the formation of the linoleic acid hydroperoxide. Briefly, R1 was incubated in buffer containing 3 mM Deoxycholate (DOC) in the presence of 0.26 mM phospholipase substrate (Sigma) and 12,226 units/ml of lipoxygenase (Sigma) in a final volume of 100 μl. Spectrophotometric measurements were performed using a Beckman DU 640 spectrophotometer (Beckman Coulter, Inc.) The PLA activity, expressed as the rate of product formed, ΔAbs₂₃₄/min was obtained using software from the spectrophotometer and rates were plotted using Microsoft Excel Software.

Transient Transfection of Plasmid DNA into COS7 Cells, and RGC Retinal Cell.

Cells were grown in 6-well plates in medium without antibiotics for 24 hours before transfecting them with 4 pg of purified plasmid DNA using Lipofectamine™ (Invitrogen) following manufacturer's instructions. After 24-48 hours post-transfection, cells were harvested for protein analysis or labeled with Lumio Green Labeling Reagent TM (Invitrogen) for immunofluorescence analysis following manufacturer's instructions. Cells transfected with vector without Lumio Tag or with pcLumio/p62 were used as negative and positive controls respectively. Protein p62 localizes to the nucleolus.

### Binding Assays for Lumio-R1 and PEDF.

After cell fractionation, the cytosolic fraction of R1N-transfected COS-7 cells was mixed with PEDF in a total volume of 140 μl, using about 170 μg cytosolic protein and 0.5 μg PEDF. Reactions were also conducted using about 340 μg cytosolic protein or 2 μg PEDF. Reactions were incubated at 4° C. for 1 hour or 16 hours and then applied to Centricon-100 filtration devices (Amicon). 3 washes were performed with 2 ml homogenization buffer for 45 min. at 1000×g, 4° C. Retained PEDF-R1 complexes were recovered by inverting the Centricon and spinning at 500×g and collecting in a retanate tube. Complexes were analyzed by 10-20% SDS-PAGE followed by Lumio detection of the R1 protein and Western blotting against monoclonal Anti-PEDF.

R1 Polypeptides Alignment with Collagen I.

PEDF binding region, p12 and R1 polypeptide sequences were aligned to human Collagen I (alpha chain) sequence to find similarities using SIM-LALNVIEW software. Multiple alignment were performed considering a maximum of 20 significative alignments an a BLOSUM62 comparison matrix (Duret et al., CABIOS, 12, 507-510, 1996). Stretches of aminoacids of p12 or R1 with significative length were aligned to the human collagen a I aminoacidic sequence, and alignment with similarity above a threshold of 25% were considered.

### Preparation of Lumio R1Plasmids.

The Gateway LR Clonase Enzyme mix (Invitrogen) was used, using pENTR vectors (Invitrogen) that contained the R1 gene and pLumio vectors (Invitrogen) that encoded for Lumio sequences at either the N or C terminal end of the inserted protein. 200 ng of pENTR vectors were used and 300 ng of Lumio vectors were used. The recombination reaction took place as shown. Nucleotide sequences of the vectors were confirmed by standard methods.

### Propogation and Purification of Lumio R1 Plasmids.

DH5α competent bacterial cells were transformed with pLumio-R1 plasmids by heat-shock. They were plated on LB/100 μg Ampicillin/ml plates and incubated at 37° C. 16 hrs. Individual colonies were picked and used to inoculate 50 ml cultures of LB/100 μg Ampicillin/ml, which were grown 16 hrs. at 37° C. The Qiagen HiSpeed Plasmid Purification Midi Kit was used to purify the plasmids from the bacterial cells, following manufacturer's instructions. Plasmids were concentrated by ethanol precipitation.

### Transfection of Mammalian Cells.

Lumio plasmids (24 μg DNA) for transfection were mixed with 60 μl of Lipofectamine 2000 (Invitrogen) diluted in Opti-MEM Reduced Serum Media and allowed to form complexes. The DNA-Lipofectamine complexes were mixed with growth media without antibiotics (Dulbecco's Modified Eagle Medium, 10% Fetal Bovine Serum) and added to mammalian cells (COS-7 or RGC-5) growing in 10 cm plates in media without antibiotics at 90% or greater confluency. Cells were harvested or Lumio labeled 24 to 48 hours after transfection.

### Lumio In-Cell Labelling of Transfected Cells.

A 2.5 μM Lumio Green Reagent solution (Invitrogen) was prepared in Opti-MEM. Growth media was removed from the cells and the Lumio solution was added and incubated for 30 minutes protected from light. The Lumio dilution was removed, and replaced with a 20 μM solution of Disperse Blue 3 in Opti-MEM. The labeled cells were visualized using fluorescence or confocal microscopy.

### Harvesting and Fractionating Transfected Cells.

Transfected cells were harvested by adding 4 ml Cell Stripper solution (Cellgro) and by using a cell scraper to remove cells from the growth surface. Fractionation was performed as described in Aymerich et al., 2001. The harvested cells were washed by centrifugation with PBS and resuspended in homogenization buffer (0.1 M KCl, 20 mM HEPES, pH 7, with protease inhibitors). After homogenization and sonication, some of the cell lysate was saved for further analysis. Debris was separated by centrifugation at 1500×g. The soluble material was further centrifuged at 150,000×g to fractionate plasma membranes in the pellet. The supernatant was removed and labeled as the Cytosolic fraction. The pellet was resuspended in SDS-sample buffer and labeled as the Membrane fraction.

### Lumio In-Gel Visualization.

Fractionated cell samples were prepared for SDS-PAGE using the Lumio Green Labeling Kit (Invitrogen) and following the manufacturer's instructions. Lumio Green reagent was added to each sample, followed by the Lumio Enhancer. After electrophoresis, the Lumio-labeled proteins in the gel were visualized on a Typhoon 9410 laser based scanner (Amersham) using a Green 532 nm laser and a 555 nm bandpass filter.

### Cell Culture.

Cell lines BHK (baby hamster kidney), HUVEC (human umbilical cord vein endothelial cells), ARPE19 (adult retinal pigment epithelium), mouse NIH 3T3-L1 preadipocytes, mouse NIH 3T3 fibroblasts, RGC-5 (rat retinal ganglion cell line), and R28 (rat retinal culture) were grown in media as recommended by ATCC. Cells harvested for expression assays were grown to confluence in T-75 flasks and 6-well plates. PEDF modulation of PEDF-R1 was assayed by culturing cells in 6-well plates to confluence in media with 10% fetal bovine serum. Cells were then deprived of serum and cultured with 50 nM PEDF and without treatment (negative control).

RNA Isolation and cDNA synthesis.

Total RNA was extracted (Qiagen RNeasy kit) following manufacturer's instructions. The RNA was treated with DNase (Ambion TURBO DNase kit) to remove genomic DNA contamination. An oligo(dT) probe was used to reverse-transcribe mRNA from cell samples in a final volume of 20 μl using SuperScript First-Strand Synthesis System (Invitrogen) following manufacturer's instructions.

### Northern Analysis.

The Northern was performed following the instructions by Ambion for the Northern Max Gly gel, photographed under UV light or visualized by the Typhoon 9410 green laser before transfer to visualize RNA in gel, and transferred to a positively-charged BrightStar membrane. The probe was prepared by Psolaren-Biotin labeling of a p12 PCR fragment following manufacturer's instructions (Ambion). Prehybridization, hybridization and washes were performed using the BrightStar Nonisotopic Detection kit (Ambion).

### Polymerase Chain Reaction (PCR).

Primers were designed using Primer3 computer program and synthesized commercially (Invitrogen). The reverse-transcription PCR reaction was performed using PCR SuperMix (Invitrogen) in a thermocycler (PE) in a final volume of 50 μl containing 2 μl cDNA and 10 μM primers specific for the R1 gene with GADPH and 18S primers as a positive control in the following manner: initial denaturation at 94° C., 35 cycles of 15s at 94° C., 30s at 61° C., and 45s at 72° C., and 5 minutes at 72° C. PCR products were resolved by agarose gel electrophoresis and visualized with ethidium bromide.

### QRT-PCR.

Quantitative real-time PCR was performed on cDNA samples as described before by Martinez et al. in an Opticon cycler (MJ Research) using SYBR Green PCR master mix (Applied Biosystems) following manufacturer's instructions. The reactions were performed in a final volume of 25 μl with 2 μl cDNA and 10 μM species-specific PEDF and PEDF-R1 primers, and 18S primers to normalize the target genes. Amplification was run by the following protocol: initial denaturation at 95° C., 45 cycles of 30s at 95° C., 30s at 60° C., and 30s at 72° C. Fluorescence was measure in every cycle and a melting curve was performed at the end of the run by increasing the temperature from 50° C. to 96° C. (0.5° C. increments) to confirm the amplification of a single product. Relative expression was determined by dividing the values by those of the 18S and were plotted using Excel.

1. An isolated PEDF-R polynucleotide, wherein said polynucleotide is
(a) a polynucleotide that has the sequence of SEQ ID NO: 1, 2 or 4;
(b) a polynucleotide that hybridizes under stringent hybridization conditions to (a) and encodes a polypeptide having the sequence of SEQ ID NO: 3 or 5; or
(c) a polynucleotide that hybridizes under stringent hybridization conditions to (a) and encodes a polypeptide with at least 25 contiguous residues of the polypeptide of SEQ ID NO: 3 or 5; or
(d) a polynucleotide that hybridizes under stringent hybridization conditions to (a) and has at least 12 contiguous bases identical to or exactly complementary to SEQ ID NO: 1, 2, or 4,
wherein the polynucleotide encodes a polypeptide having PEDF-R activity.

2. An isolated PEDF-R polynucleotide, wherein said polynucleotide is
(a) a polynucleotide that has the sequence of SEQ ID NO: 12, 13, 15, or 16;
(b) a polynucleotide that hybridizes under stringent hybridization conditions to (a) and encodes a polypeptide having the sequence of SEQ ID NO: 14 or 17; or
(c) a polynucleotide that hybridizes under stringent hybridization conditions to (a) and encodes a polypeptide with at least 25 contiguous residues of the polypeptide of SEQ ID NO: 14 or 17; or
(d) a polynucleotide that hybridizes under stringent hybridization conditions to (a) and has at least 12 contiguous bases identical to or exactly complementary to SEQ ID NO: 12, 13, 15, or 16,
wherein the polynucleotide encodes a polypeptide having PEDF-R activity.

3. An isolated PEDF-R polynucleotide encoding a polypeptide comprising a sequence at least 60% identical to SEQ ID NO:3 and having PEDF-R activity.

4. An isolated PEDF-R polynucleotide encoding a polypeptide comprising a sequence at least 60% identical to SEQ ID NO:5 and having PEDF-R activity

5. The isolated PEDF-R polynucleotide of claim 1 encoding a polypeptide comprising the sequence of SEQ ID NO:3 or 5.

6. The PEDF-R polynucleotide of claim 1 encoding a polypeptide having a binding affinity of at least 10⁴ M⁻¹ for binding PEDF.

7. The PEDF-R polynucleotide of claim 1 comprising SEQ ID NO: 1 or its complement.

8. The PEDF-R polynucleotide of claim 1 comprising SEQ ID NO:2 or its complement.

9. The PEDF-R polynucleotide of claim 1 comprising SEQ ID NO:4 or its complement.

10. A nucleic acid comprising the cDNA coding sequence of ATCC Deposit No. accession number BC017280.1, accession number XM_{—}341960.1, or accession number AK031609.1.

11. A polypeptide comprising the amino acid sequence of ATCC Deposit No. accession number BAC27476.1, accession number XP_{—}341961.1, or accession number AAH17280.1.

12. An isolated polynucleotide comprising a nucleotide sequence having at least 60% identity to SEQ ID NO: 1, 2 or 4 or a complement thereof and having PEDF-R activity.

13. An isolated polypeptide comprising a nucleotides sequence that has at least 90% sequence identity to SEQ ID NO:3 or SEQ ID NO:5 and is immunologically cross-reactive with SEQ ID NO:3 or SEQ ID NO:5 or shares a biological function with native PEDF-R.

14. A vector comprising the isolated PEDF-R polynucleotide of claim 1.

15. An expression vector comprising the PEDF-R polynucleotide of claim 1 operatively linked to a regulatory sequence that controls expression of the polynucleotide in a host cell.

16. The expression vector of claim 15 wherein the polynucleotide is operatively linked to the regulatory sequence in an antisense orientation.

17. The expression vector of claim 15 wherein the polynucleotide is operatively linked to the regulatory sequence in a sense orientation.

18. A host cell comprising the polynucleotide of claim 1, or progeny of the cell.

19. The host cell of claim 18 which is a eukaryote.

20. A host cell comprising the polynucleotide of claim 1 operatively linked with a regulatory sequence that controls expression of the polynucleotide in a host cell.

21. The host cell of claim 20 wherein the nucleic acid is operatively linked to the regulatory sequence in an antisense orientation.

22. The expression vector of claim 20 wherein the nucleic acid is operatively linked to the regulatory sequence in a sense orientation.

23. An isolated DNA that encodes a PEDF-R protein as shown in SEQ ID NO:3 or 5.

24. An antisense oligonucleotide complementary to a messenger RNA comprising SEQ ID NO: 1, 2, or 4 and encoding PEDF-R, wherein the oligonucleotide inhibits the expression of PEDF-R.

25. The polynucleotide of claim 1 that is RNA.

26. A method of producing a polypeptide comprising:
(i) culturing the host cell of claim 18 under conditions such that the polypeptide is expressed; and
(ii) recovering the polypeptide from the cultured host cell of its cultured medium.

27. An isolated polypeptide encoded by a polynucleotide of claim 1(a) or (b).

28. The polypeptide of claim 27 that has the amino acid sequence of SEQ ID NO:3 or 5.

29. An isolated polypeptide having 60% sequence identity to the amino acid sequence of SEQ ID NO:5 and having PEDF-R activity

30. The polypeptide of claim 29 comprising SEQ ID NO:3.

31. The polypeptide of claim 29 comprising SEQ ID NO:5.

32. The isolated polypeptide of claim 27 that is cell-membrane associated.

33. The isolated polypeptide of claim 27 that is soluble.

34. The isolated polypeptide of claim 27 that is fused with a heterologous peptide.

35. An isolated antibody that specifically binds to a polypeptide having the amino acid sequence as shown in SEQ ID NO:3 or SEQ ID NO:5.

36. An isolated antibody composition that specifically binds to a polypeptide of claim 27.

37. The isolated antibody composition of claim 35 that is monoclonal.

38. The isolated antibody composition of claim 35 that is polyclonal.

39. The isolated antibody of claims 37 or 38 that is labeled.

40. The isolated antibody of claims 37 or 38 that is conjugated to a toxic or non-toxic moiety.

41. The isolated antibody composition of claims 37 or 38 that is a neutralizing antibody.

42. A hybridoma capable of secreting the antibody that binds to a polypeptide of claim 37.

43. A method for identifying a compound or agent that binds to a PEDF-R polypeptide comprising:
(i) contacting a PEDF receptor polypeptide of claim 27 with the compound or agent under conditions which allow binding of the compound to the PEDF-R polypeptide to form a complex and
(ii) detecting the presence of the complex.

44. A method of detecting a PEDF-R polypeptide in a sample, comprising:
(i) contacting the sample with an antibody of claim 37, and
(ii) determining whether a hybridization complex has been formed between the antibody and the PEDF-R polypeptide.

45. A method of detecting a PEDF-R polypeptide in a sample, comprising:
(i) contacting the sample with a polynucleotide of claim 1 or a polynucleotide that comprises a sequence of at least 12 nucleotides and is complementary to a contiguous sequence of the polynucleotide of section (a) of claim 1; and
(ii) determining whether a hydridization complex has been formed.

46. The method of claim 45, wherein said method is used to diagnose a disease or disorder of the nervous system.

47. The method of claim 45, wherein said method is used to diagnose a disease or disorder associated with angiogenesis.

48. The method of claim 45, wherein said method is used to diagnose an ocular disease or disorder.

49. A method of detecting a PEDF-R nucleotide in a sample, comprising:
(i) using a polynucleotide that comprises a sequence of at least 12 nucleotides and is complementary to a contiguous sequence of a polynucleotide of section (a) of claim 1, in an amplification process, and
(ii) determining whether a specific amplification product has been formed.

50. The method of claim 49, wherein said method is used to diagnose a disease or disorder of the nervous system.

51. The method of claim 49, wherein said method is used to diagnose a disease or disorder associated with angiogenesis.

52. The method of claim 49, wherein said method is used to diagnose an ocular disease or disorder.

53. A pharmaceutical composition comprising a polynucleotide of claim 1, or a polypeptide of claim 27 or an antibody of claim 35 and a pharmaceutically acceptable carrier.

54. A pharmaceutical composition comprising an antibody of claim 35.

55. A method of modulating PEDF activity, comprising
(i) modulating with the expression of a PEDF-R gene;
(ii) modulating the ability of a PEDF-R protein to bind to another cell; or
(iii) modulating the ability of a PEDF-R protein to bind to another protein.

56. A method of modulating PEDF activity in a subject, comprising administering to the subject a therapeutically effective amount of the pharmaceutical composition of claim 53.

57. The method of claim 56 wherein the PEDF activity is neurotrophic, neuronotrophic, gliastatic, anti-angiogenic, or adipostatic.

58. The method of claim 56 wherein the PEDF activity is the inhibition of ocular angiogenesis or neovascularizaton.

59. The method of claim 58 wherein the ocular angiogenesis is caused by ischemia.

60. The method of claim 56 wherein the PEDF activity is the inhibition of retinal cell degeneration.

61. A method of treating a disease or disorder in a subject comprising administering to the subject a therapeutically effective amount of the pharmaceutical composition of claim 53, wherein the disease or disorder is neurological or ocular.

62. (canceled)

63. The method of claim 61, wherein the disease or disorder is macular degeneration.


SEQUENCE LISTING (35 sequences)

SEQ ID NO: 1 - DNA, 2122 bp, Homo sapiens
ggcacgagggcggccccagtcagacgcaggcagccccaaagcctgaacaggcagggccagacccagcttcttcgcctccgccagcggggaccccgagctagagccgcagcgggacctgcccggcccccggctccagcgagcgagcggcgagcaggcggctcacagaggcctggccgcccacggaacccggggcccggcggccgccgccgcgatgtttccccgcgagaagacgtggaacatctcgttcgcgggctgcggcttcctcggcgtctactacgtcggcgtggcctcctgcctccgcgagcacgcgcccttcctggtggccaacgccacgcacatctacggcgcctcggccggggcgctcacggccacggcgctggtcaccggggtctgcctgggtgaggctggtgccaagttcattgaggtatctaaagaggcccggaagcggttcctgggccccctgcacccctccttcaacctggtaaagatcatccgcagtttcctgctgaaggtcctgcctgctgatagccatgagcatgccagtgggcgcctgggcatctccctgacccgcgtgtcagacggcgagaatgtcattatatcccacttcaactccaaggacgagctcatccaggccaatgtctgcagcggtttcatccccgtgtactgtgggctcatccctccctccctccagggggtgcgctacgtggatggtggcatttcagacaacctgccactctatgagcttaagaacaccatcacagtgtcccccttctcgggcgagagtgacatctgtccgcaggacagctccaccaacatccacgagctgcgggtcaccaacaccagcatccagttcaacctgcgcaacctctaccgcctctccaaggccctcttcccgccggagcccctggtgctgcgagagatgtgcaagcagggataccgggatggcctgcgctttctgcagcggaacggcctcctgaaccggcccaaccccttgctggcgttgccccccgcccgcccccacggcccagaggacaaggaccaggcagtggagagcgcccaagcggaggattactcgcagctgccgggagaagatcacatcctggagcacctgcccgcccggctcaatgaggccctgctggaggcctgcgtggagcccacggacctgctgaccaccctctccaacatgctgcctgtgcgtctggccacggccatgatggtgccctacacgctgccgctggagagcgctctgtccttcaccatccgcttgctggagtggctgcccgacgttcccgaggacatccggtggatgaaggagcagacgggcagcatctgccagtacctggtgatgcgcgccaagaggaagctgggcaggcacctgccctccaggctgccggagcaggtggagctgcgccgcgtccagtcgctgccgtccgtgccgctgtcctgcgccgcctacagagaggcactgcccggctggatgcgcaacaacctctcgctgggggacgcgctggccaagtgggaggagtgccagcgccagctgctgctcggcctcttctgcaccaacgtggccttcccgcccgaagctctgcgcatgcgcgcacccgccgacccggctcccgcccccgcggacccagcatccccgcagcaccagctggccgggcctgcccccttgctgagcacccctgctcccgaggcccggcccgtgatcggggccctggggctgtgagaccccgaccctctcgaggaaccctgcctgagacgcctccattaccactgcgcagtgagatgaggggactcacagttgccaagaggggtctttgccgtgggccccctcgccagccactcaccagctgcatgcactgagaggggaggtttccacacccctcccctgggccgctgaggccccgcgcacctgtgccttaatcttccctcccctgtgctgcccgagcacctcccccgcccctttactcctgagaactttgcagctgcccttccctccccgtttttcatggcctgctgaaatatgtgtgtgaagaattatttattttcgccaaagcacatgtaataaatgctgcagcccaaaaaaaaaaaaaaaaaaaaaaaaaaaaaaaaaaaaaaaaaa

SEQ ID NO: 2 - DNA, 1515 bp, Homo sapiens
atgtttccccgcgagaagacgtggaacatctcgttcgcgggctgcggcttcctcggcgtctactacgtcggcgtggcctcctgcctccgcgagcacgcgcccttcctggtggccaacgccacgcacatctacggcgcctcggccggggcgctcacggccacggcgctggtcaccggggtctgcctgggtgaggctggtgccaagttcattgaggtatctaaagaggcccggaagcggttcctgggccccctgcacccctccttcaacctggtaaagatcatccgcagtttcctgctgaaggtcctgcctgctgatagccatgagcatgccagtgggcgcctgggcatctccctgacccgcgtgtcagacggcgagaatgtcattatatcccacttcaactccaaggacgagctcatccaggccaatgtctgcagcggtttcatccccgtgtactgtgggctcatccctccctccctccagggggtgcgctacgtggatggtggcatttcagacaacctgccactctatgagcttaagaacaccatcacagtgtcccccttctcgggcgagagtgacatctgtccgcaggacagctccaccaacatccacgagctgcgggtcaccaacaccagcatccagttcaacctgcgcaacctctaccgcctctccaaggccctcttcccgccggagcccctggtgctgcgagagatgtgcaagcagggataccgggatggcctgcgctttctgcagcggaacggcctcctgaaccggcccaaccccttgctggcgttgccccccgcccgcccccacggcccagaggacaaggaccaggcagtggagagcgcccaagcggaggattactcgcagctgccgggagaagatcacatcctggagcacctgcccgcccggctcaatgaggccctgctggaggcctgcgtggagcccacggacctgctgaccaccctctccaacatgctgcctgtgcgtctggccacggccatgatggtgccctacacgctgccgctggagagcgctctgtccttcaccatccgcttgctggagtggctgcccgacgttcccgaggacatccggtggatgaaggagcagacgggcagcatctgccagtacctggtgatgcgcgccaagaggaagctgggcaggcacctgccctccaggctgccggagcaggtggagctgcgccgcgtccagtcgctgccgtccgtgccgctgtcctgcgccgcctacagagaggcactgcccggctggatgcgcaacaacctctcgctgggggacgcgctggccaagtgggaggagtgccagcgccagctgctgctcggcctcttctgcaccaacgtggccttcccgcccgaagctctgcgcatgcgcgcacccgccgacccggctcccgcccccgcggacccagcatccccgcagcaccagctggccgggcctgcccccttgctgagcacccctgctcccgaggcccggcccgtgatcggggccctggggctgtga

SEQ ID NO: 3 - protein, 504 aa, Homo sapiens
MFPREKTWNISFAGCGFLGVYYVGVASCLREHAPFLVANATHIYGASAGALTATALVTGVCLGEAGAKFIEVSKEARKRFLGPLHPSFNLVKIIRSFLLKVLPADSHEHASGRLGISLTRVSDGENVIISHFNSKDELIQANVCSGFIPVYCGLIPPSLQGVRYVDGGISDNLPLYELKNTITVSPFSGESDICPQDSSTNIHELRVTNTSIQFNLRNLYRLSKALFPPEPLVLREMCKQGYRDGLRFLQRNGLLNRPNPLLALPPARPHGPEDKDQAVESAQAEDYSQLPGEDHILEHLPARLNEALLEACVEPTDLLTTLSNMLPVRLATAMMVPYTLPLESALSFTIRLLEWLPDVPEDIRWMKEQTGSICQYLVMRAKRKLGRHLPSRLPEQVELRRVQSLPSVPLSCAAYREALPGWMRNNLSLGDALAKWEECQRQLLLGLFCTNVAFPPEALRMRAPADPAPAPADPASPQHQLAGPAPLLSTPAPEARPVIGALGL

SEQ ID NO: 4 - DNA, 404 bp, Homo sapiens
cagcggaacggcctcctgaaccggcccaaccccttgctggcgttgccccccgcccgcccccacggcccagaggacaaggaccaggcagtggagagcgcccaagcggaggattactcgcagctgccgggagaagatcacatcctggagcacctgcccgcccggctcaatgaggccctgctggaggcctgcgtggagcccacggacctgctgaccaccctctccaacatgctgcctgtgcgtctggccacggccatgatggtgccctacacgctgccgctggagagcgctctgtccttcaccatccgcttgctggagtggctgcccgacgttcccgaggacatccggtggatgaaggagcagacgggcagcatctgccagtacctggtgatgcgcgccaagaggaa

SEQ ID NO: 5 - protein, 134 aa, Homo sapiens
QRNGLLNRPNPLLALPPARPHGPEDKDQAVESAQAEDYSQLPGEDHILEHLPARLNEALLEACVEPTDLLTTLSNMLPVRLATAMMVPYTLPLESALSFTIRLLEWLPDVPEDIRWMKEQTGSICQYLVMRAKR

SEQ ID NO: 6 - DNA, 29 bp, Artificial
  Primer 1 for the construction of p12
caccatgcagcggaacggcctcctgaacc

SEQ ID NO: 7 - DNA, 25 bp, Artificial
  Primer 2 for the construction of p12
ctagttcctcttggcgcgcatcacc

SEQ ID NO: 8 - DNA, 22 bp, Artificial
  Primer 3 for the construction of p12
gttcctcttggcgcgcatcacc

SEQ ID NO: 9 - DNA, 25 bp, Artificial
  Primer 11 for the construction of R1 expression vectors
ccacatgtttccccgcgagaagacg

SEQ ID NO: 10 - DNA, 25 bp, Artificial
  Primer 12 for the construction of R1 expression vectors
ctacagccccagggccccgatcacg

SEQ ID NO: 11 - DNA, 22 bp, Artificial
  Primer 13 for the construction of R1 expression vectors
cagccccagggccccgatcacg

SEQ ID NO: 12 - DNA, 1965 bp, Mus musculus
ggagaccccaaggtatcgagactgcgggacccactgcccgcaggacatcgagtcacgatgttcccgagggagaccaagtggaacatctcattcgctggctgcggcttcctcggggtctaccacattggcgtggcctcctgcctccgtgagcacgcgcccttcctggtggccaacgccactcacatctacggagcctcggcaggggcgctcaccgccacagcgctggtcactggggcctgcctgggtgaagcaggtgccaacattattgaggtgtccaaggaggcccggaagcggttcctgggtcctctgcatccctccttcaacctggtgaagaccatccgtggctgtctactaaagaccctgcctgctgattgccatgagcgcgccaatggacgcctgggcatctccctgactcgtgtttcagacggagagaacgtcatcatatcccactttagctccaaggatgagctcatccaggccaatgtctgcagcacatttatcccggtgtactgtggcctcattcctcctaccctccaaggggtgcgctatgtggatggcggcatttcagacaacttgccactttatgagctgaagaataccatcacagtgtccccattctcaggcgagagtgacatctgccctcaggacagctccaccaacatccacgagcttcgcgtcaccaacaccagcatccagttcaaccttcgcaatctctaccgcctctcgaaggctctcttcccgccagagcccatggtcctccgagagatgtgcaaacagggctacagagatggacttcgattccttaggaggaatggcctactgaaccaacccaaccctttgctggcactgcccccagttgtcccccaggaagaggatgcagaggaagctgctgtggtggaggagagggctggagaggaggatcaattgcagccttatagaaaagatcgaattctagagcacctgcctgccagactcaatgaggccctgctggaggcctgtgtggaaccaaaggacctgatgaccaccctttccaacatgctaccagtgcgcctggcaacggccatgatggtgccctatactctgccgctggagagtgcagtgtccttcaccatccgcttgttggagtggctgcctgatgtccctgaagatatccggtggatgaaagagcagacgggtagcatctgccagtatctggtgatgagggccaagaggaaattgggtgaccatctgccttccagactgtctgagcaggtggaactgcgacgtgcccagtctctgccctctgtgccactgtcttgcgccacctacagtgaggccctacccaactgggtacgaaacaacctctcactgggggacgcgctggccaagtgggaagaatgccagcgtcagctactgctgggtctcttctgcaccaatgtggccttcccgccggatgccttgcgcatgcgcgcacctgccagccccactgccgcagatcctgccaccccacaggatccacctggcctcccgccttgctgagaatcaccattcccacatcgcccggctaccagccaagctccaagttgtcctgccccactaagaggagccccggggtggaacaagatcctgtctgccccggctctcccccttacatgctgtggaatgaggacataggaccctgcacagctgcaagtgggctttcgatgtgaaacctttcaccagccactcactatgctactcctggtggggagggatggggagtcgccctcccccggagcccacagagccctcccccgtcacgtcacctgtgccttactcctgcccaccaccttttcagtgcagggtcagtcttaagaactccacatctgctgctgctccctggtgtccaagtttccttgcagagtgtgtgaagaattatttatttttgccaaagcagatctaataaaagccacagctcagcttctgccttcctcacttctgcatgct

SEQ ID NO: 13 - DNA, 1461 bp, Mus musculus
atgttcccgagggagaccaagtggaacatctcattcgctggctgcggcttcctcggggtctaccacattggcgtggcctcctgcctccgtgagcacgcgcccttcctggtggccaacgccactcacatctacggagcctcggcaggggcgctcaccgccacagcgctggtcactggggcctgcctgggtgaagcaggtgccaacattattgaggtgtccaaggaggcccggaagcggttcctgggtcctctgcatccctccttcaacctggtgaagaccatccgtggctgtctactaaagaccctgcctgctgattgccatgagcgcgccaatggacgcctgggcatctccctgactcgtgtttcagacggagagaacgtcatcatatcccactttagctccaaggatgagctcatccaggccaatgtctgcagcacatttatcccggtgtactgtggcctcattcctcctaccctccaaggggtgcgctatgtggatggcggcatttcagacaacttgccactttatgagctgaagaataccatcacagtgtccccattctcaggcgagagtgacatctgccctcaggacagctccaccaacatccacgagcttcgcgtcaccaacaccagcatccagttcaaccttcgcaatctctaccgcctctcgaaggctctcttcccgccagagcccatggtcctccgagagatgtgcaaacagggctacagagatggacttcgattccttaggaggaatggcctactgaaccaacccaaccctttgctggcactgcccccagttgtcccccaggaagaggatgcagaggaagctgctgtggtggaggagagggctggagaggaggatcaattgcagccttatagaaaagatcgaattctagagcacctgcctgccagactcaatgaggccctgctggaggcctgtgtggaaccaaaggacctgatgaccaccctttccaacatgctaccagtgcgcctggcaacggccatgatggtgccctatactctgccgctggagagtgcagtgtccttcaccatccgcttgttggagtggctgcctgatgtccctgaagatatccggtggatgaaagagcagacgggtagcatctgccagtatctggtgatgagggccaagaggaaattgggtgaccatctgccttccagactgtctgagcaggtggaactgcgacgtgcccagtctctgccctctgtgccactgtcttgcgccacctacagtgaggccctacccaactgggtacgaaacaacctctcactgggggacgcgctggccaagtgggaagaatgccagcgtcagctactgctgggtctcttctgcaccaatgtggccttcccgccggatgccttgcgcatgcgcgcacctgccagccccactgccgcagatcctgccaccccacaggatccacctggcctcccgccttgctga

SEQ ID NO: 14 - protein, 486 aa, Mus musculus
MFPRETKWNISFAGCGFLGVYHIGVASCLREHAPFLVANATHIYGASAGALTATALVTGACLGEAGANIIEVSKEARKRFLGPLHPSFNLVKTIRGCLLKTLPADCHERANGRLGISLTRVSDGENVIISHFSSKDELIQANVCSTFIPVYCGLIPPTLQGVRYVDGGISDNLPLYELKNTITVSPFSGESDICPQDSSTNIHELRVTNTSIQFNLRNLYRLSKALFPPEPMVLREMCKQGYRDGLRFLRRNGLLNQPNPLLALPPVVPQEEDAEEAAVVEERAGEEDQLQPYRKDRILEHLPARLNEALLEACVEPKDLMTTLSNMLPVRLATAMMVPYTLPLESAVSFTIRLLEWLPDVPEDIRWMKEQTGSICQYLVMRAKRKLGDHLPSRLSEQVELRRAQSLPSVPLSCATYSEALPNWVRNNLSLGDALAKWEECQRQLLLGLFCTNVAFPPDALRMRAPASPTAADPATPQDPPGLPPC

SEQ ID NO: 15 - DNA, 1533 bp, Rattus sp.
tcctctgcctcccggcacagcgtctccgcctccgccggcggggaccccaggttatcaagactgcgggacccactgcccgcaggacgtctaatcacgatgttcccaagggagaccaagtggaacatctcgttcgctggctgcggcttcctcggggtctaccacattggagtggcctcctgcctccgtgagcacgcgcccttcctggtggccaacgccactcacatctacggagcctcggcaggggcgcttaccgccacagcgctggtcactggggcctgcctgggcgaagcgggtgccaacattattgaggtgtccaaggaggctcggaagcggttcctgggtcccctgcacccctccttcaacctggtaaagaccatccgtggttgtctactgaagaccctgcctgctgattgccacacgcgtgccagcggacgcctgggcatctccctgactcgagtttcggatggagagaatgtcatcatatcgcactttagctccaaggatgagcttatccaggccaatgtttgcagcacttttatccctgtgtactgtggcctcattcctcctacccttcaaggggtgcgctatgtggatggcggcatttcagacaacttgccactttatgagctgaagaataccatcacagtgtccccattctcaggcgagagtgacatctgcccacaagacagctccaccaacatccacgaacttcgtatcaccaacaccagcatccaattcaacctgcgcaatctctaccgcctctcgaaggctctcttcccgccagagcccatggttctccgagagatgtgcaaacagggctaccgagatggacttcgattccttaggaggaatggcctactgaaccaacccaaccctttgctggcactgcccccggttgtcccccaggaagaggatgcagaggaagctgccgtgactgaggagaggactggaggggaggatcggattctagagcacctgcctgccagactcaacgaggccctgctggaggcctgtgtggaaccgaaagacctgatgaccaccctttccaacatgctgccagtgcgcctggccactgccatgatggtaccctatactctgccactggagagcgcagtgtccttcaccatccgtttgttggagtggctgcctgatgtccctgaggatatccggtggatgaaggagcagacaggtagcatctgccagtatctggtgatgagggccaagaggaaattgggtgaccatctaccttccagactgtctgagcaggtggagctgcggcgtgcccagtctctgccgtctgtgccactgtcttgcgccacctacagtgaggcactgcccaactgggtacgaaacaacctctcactgggggacgcgctggccaagtgggaagaatgccagcgtcagctactgctgggtctcttctgcaccaatgtggccttcccgcctgatgccttgcgcatgcgcgcacctgccagccccaccgccacagatcctgccaccccacaggatccatctggcctcccaccttgctga

SEQ ID NO: 16 - DNA, 1437 bp, Rattus sp.
atgttcccaagggagaccaagtggaacatctcgttcgctggctgcggcttcctcggggtctaccacattggagtggcctcctgcctccgtgagcacgcgcccttcctggtggccaacgccactcacatctacggagcctcggcaggggcgcttaccgccacagcgctggtcactggggcctgcctgggcgaagcgggtgccaacattattgaggtgtccaaggaggctcggaagcggttcctgggtcccctgcacccctccttcaacctggtaaagaccatccgtggttgtctactgaagaccctgcctgctgattgccacacgcgtgccagcggacgcctgggcatctccctgactcgagtttcggatggagagaatgtcatcatatcgcactttagctccaaggatgagcttatccaggccaatgtttgcagcacttttatccctgtgtactgtggcctcattcctcctacccttcaaggggtgcgctatgtggatggcggcatttcagacaacttgccactttatgagctgaagaataccatcacagtgtccccattctcaggcgagagtgacatctgcccacaagacagctccaccaacatccacgaacttcgtatcaccaacaccagcatccaattcaacctgcgcaatctctaccgcctctcgaaggctctcttcccgccagagcccatggttctccgagagatgtgcaaacagggctaccgagatggacttcgattccttaggaggaatggcctactgaaccaacccaaccctttgctggcactgcccccggttgtcccccaggaagaggatgcagaggaagctgccgtgactgaggagaggactggaggggaggatcggattctagagcacctgcctgccagactcaacgaggccctgctggaggcctgtgtggaaccgaaagacctgatgaccaccctttccaacatgctgccagtgcgcctggccactgccatgatggtaccctatactctgccactggagagcgcagtgtccttcaccatccgtttgttggagtggctgcctgatgtccctgaggatatccggtggatgaaggagcagacaggtagcatctgccagtatctggtgatgagggccaagaggaaattgggtgaccatctaccttccagactgtctgagcaggtggagctgcggcgtgcccagtctctgccgtctgtgccactgtcttgcgccacctacagtgaggcactgcccaactgggtacgaaacaacctctcactgggggacgcgctggccaagtgggaagaatgccagcgtcagctactgctgggtctcttctgcaccaatgtggccttcccgcctgatgccttgcgcatgcgcgcacctgccagccccaccgccacagatcctgccaccccacaggatccatctggcctcccaccttgctga

SEQ ID NO: 17 - protein, 478 aa, Rattus sp.
MFPRETKWNISFAGCGFLGVYHIGVASCLREHAPFLVANATHIYGASAGALTATALVTGACLGEAGANIIEVSKEARKRFLGPLHPSFNLVKTIRGCLLKTLPADCHTRASGRLGISLTRVSDGENVIISHFSSKDELIQANVCSTFIPVYCGLIPPTLQGVRYVDGGISDNLPLYELKNTITVSPFSGESDICPQDSSTNIHELRITNTSIQFNLRNLYRLSKALFPPEPMVLREMCKQGYRDGLRFLRRNGLLNQPNPLLALPPVVPQEEDAEEAAVTEERTGGEDRILEHLPARLNEALLEACVEPKDLMTTLSNMLPVRLATAMMVPYTLPLESAVSFTIRLLEWLPDVPEDIRWMKEQTGSICQYLVMRAKRKLGDHLPSRLSEQVELRRAQSLPSVPLSCATYSEALPNWVRNNLSLGDALAKWEECQRQLLLGLFCTNVAFPPDALRMRAPASPTATDPATPQDPSGLPPC

SEQ ID NO: 18 - DNA, 20 bp, Artificial
  PCR primer
gcagtttcctgctgaaggtc

SEQ ID NO: 19 - DNA, 20 bp, Artificial
  PCR primer
gctcgtccttggagttgaag

SEQ ID NO: 20 - DNA, 20 bp, Artificial
  Primer
tgtggcctcattcctcctac

SEQ ID NO: 21 - DNA, 20 bp, Artificial
  Primer
tgagaatggggacactgtga

SEQ ID NO: 22 - DNA, 20 bp, Artificial
  Primer
tatccggtggatgaaagagc

SEQ ID NO: 23 - DNA, 20 bp, Artificial
  Primer
cagttccacctgctcagaca

SEQ ID NO: 24 - protein, 9 aa, Artificial
  Synthetic Construct
DKTHTCPPC

SEQ ID NO: 25 - DNA, 19 bp, Artificial
  Forward primer
aaccccttgctggcgttgc

SEQ ID NO: 26 - DNA, 19 bp, Artificial
  Reverse primer
cccgtctgctccttcatcc

SEQ ID NO: 27 - protein, 292 aa, Artificial
  adiponutrin
YDARGSLFHATRHLRDRMLFHCVGVLSIPEQTLQVLSDLVRKSNIIFSFLQGCCNVQLIKILVDFRVVDLCFSFRVVFIDATPYYKVKFLHVDIKLLRLCTGLRFVDLKGILRLAFEEKICQGKSSSEGMDPEVAMPSWANMSLDSSSAALRLEGDLLHLRSILPWESDTSPATSEMKDKGGYMSKICLIIMSYVLCVIAIVQVTMDVLLQWVSQVFTRVLCLLPASRSQMVSSQQASPCTPEDWCWTCPKGCPAETKAEATPRSIRSSNFFLGNKVPAGAEGLSSFSEKSL

SEQ ID NO: 28 - protein, 41 aa, Artificial
  Synthetic Construct
GLLNRPNPLLALPPARPHGEPDKDQAVESAQAEDYSQLPGE

SEQ ID NO: 29 - protein, 55 aa, Artificial
  Synthetic Construct
TNVAFPPEALRMRAPADPAPAPADPASPQHQLAGPAPLLSTPAPEARPVIGALGL

SEQ ID NO: 30 - protein, 14 aa, Artificial
  R1
NATIYGASAGALTA

SEQ ID NO: 31 - protein, 15 aa, Artificial
  Patatain B2
YFDVIGGTSTGGLLT

SEQ ID NO: 32 - protein, 15 aa, Artificial
  cPLA2
CATYVAGLSGSTWYM

SEQ ID NO: 33 - protein, 20 aa, Artificial
  R1
SLQGVRYVDGGISDNLPLYE

SEQ ID NO: 34 - protein, 20 aa, Artificial
  Patatin B2
ARYEFNLVDGAVATVGDPAL

SEQ ID NO: 35 - protein, 19 aa, Artificial
  cPLA2
KSKKIHVVDSGLTFNLPYP